(19) United States
(12) Patent Application Publication
(10) Pub. No.: US 2025/0195504 A1
(43) Pub. Date: Jun. 19, 2025

(54) NALTREXAMINE DERIVATIVES BEARING 5-MEMBER HETEROCYCLIC RING SYSTEMS AND OPIOID RECEPTOR MODULATORS

(71) Applicant: VIRGINIA COMMONWEALTH UNIVERSITY, Richmond, VA (US)

(72) Inventor: Yan ZHANG, Richmond, VA (US)

(21) Appl. No.: 18/727,826

(22) PCT Filed: Feb. 22, 2023
(86) PCT No.: PCT/US2023/013588

§ 371 Date: Jul. 10, 2024

Related U.S. Application Data
(60) Provisional application No. 63/312,537, filed on Feb. 22, 2022.

(51) Int. Cl. A61K 31/485; C07D 489/08

(52) U.S. Cl. CPC A61K 31/485; C07D 489/08

(57) ABSTRACT

A series of non-peptide opioid receptor modulators having the general formula: (I) is provided. In the formula: R=(II) * is a chiral carbon; M is a saturated or unsaturated, branched or unbranched, substituted or unsubstituted alkyl chain from 0-10 atoms in length; X1, X2, X3, X4, or X5 are independently, C, N, O, or S; and R is attached to M by any of X1, X2, X3, X4, or X5. The compounds are used to treat disorders related to opioid receptor functions such as opioid addiction, opioid overdose, pain and constipation caused by opioid use.

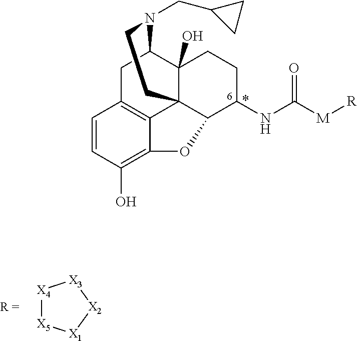

### CROSS-REFERENCE TO RELATED APPLICATIONS

This application claims benefit of U.S. provisional patent application 63/312,537, filed Feb. 22, 2022.

### STATEMENT OF FEDERALLY SPONSORED RESEARCH AND DEVELOPMENT

This invention was made with government support under grant numbers DA024022 and DA050311 awarded by the National Institutes of Health. The United States government has certain rights in the invention.

### BACKGROUND OF THE INVENTION

### Technical Field

The invention generally relates to improved non-peptide opioid receptor modulators. In particular, the invention provides non-peptide opioid receptor modulators that are potent agonists, partial agonists, or antagonists of opioid receptor and are used to treat disorders such as opioid abuse and addiction, opioid overdose, alcoholism, opioid induced constipation and irritable bowel movement disorders.

### Description of Related Art

Opioid use disorders (OUD) pose an imminent threat to human health worldwide with approximately 2.1 million Americans suffering from this epidemic. Currently, detoxification and maintenance therapy are the two most commonly used approaches to treat opioid use disorders. Methadone, buprenorphine and naltrexone (FIG. 1A) are first-line opioid medicines approved by the US Food and Drug Administration (FDA) for opioid use disorders. Methadone and buprenorphine, mu opioid receptor (MOR) full and partial agonists, respectively, show good efficacy for opioid addiction maintenance therapy. However, about 50% of patients relapse after being treated for opioid use disorders with methadone and buprenorphine. While opioid antagonists naltrexone and naloxone have displayed the ability to manage opioid misuse, overdose and reduce relapse, they carry some side effects such as dysphoria, depression and, even suicide. One of the most concerning side effects are the withdrawal symptoms precipitated by naltrexone and naloxone, including abdominal cramps, nausea/vomiting, diarrhea, muscle aches, anxiety, confusion, and extreme sleepiness. High doses of these drugs are also reported to show hepatotoxicity, cardiovascular and pulmonary problems. Some of these side effects of the opioid antagonists are related to their low selectivity to the MOR over the delta opioid receptor (DOR) and kappa opioid receptor (KOR).

Opioid receptors belong to the G-protein coupled receptors (GPCRs) super family. There are four opioid receptors viz. MOR, KOR, DOR and the nociception/orphanin FQ receptor (NOP). MOR is the primary pharmacological target of most known opioids. The overall effect of MOR activation results in lowering of postsynaptic neuronal excitability or inhibition of presynaptic neurotransmitter release. The vast array of pharmacological effects as a result of MOR activation include analgesia, euphoria, sedation, respiratory depression, cough suppression and constipation. Along with the analgesia produced by opioids, their ability to cause euphoria, due to activation of the MOR in several regions of the brain, often leads to opioid misuse. Thus, there is an urgent need to develop highly potent, efficacious and selective MOR ligands with minimum side effects as OUD medications.

At the same time, the severity of another unwanted side effect, opioid-induced constipation (OIC), is typically underestimated. This opioid-associated bowel dysfunction occurs in 40-80% of MOR agonist-treated patients and contributes to a considerable percentage of treatment cessation, causing a substantial burden to the patients and health systems. As neither dietary changes nor laxative products are effective in preventing or treating OIC, peripherally acting opioid receptor antagonists (PAMORAs) have become the major research of interest due to their targeting the underlying mechanisms of OIC, i.e., MOR receptor activation in the gastrointestinal (GI) tract. As opioid pain relievers promote analgesia mainly in supraspinal sites and the spinal cord, the peripheral selectivity is essential for potential OIC therapeutic agents to avoid compromising the analgesic effects of opioid pain relievers. In other words, PAMORAs are designed to be taken with an opioid to reverse opioid-induced constipation (OIC) without compromising the opioid's analgesic effects.

Currently, three PAMORAs have been approved by the U.S. FDA to treat OIC, namely methylnaltrexone (MNTX), naloxegol, and naldemedine (FIG. 1B). A fourth PAMORA, alvimopan, however, is indicated mainly for post-surgery gastrointestinal recovery. Their mechanisms of peripheral nervous system (PNS) restriction are derived from the chemical structures: the N-methyl quaternary amine in MNTX, the PEGylation in naloxegol, and the high molecular weight and bulky side chain in naldemedine. Such structural features limit their blood-brain barrier (BBB) permeability and restrict their MOR-antagonizing effects to the periphery. All these drugs have demonstrated sufficient efficacy and acceptable safety in a variety of clinical studies, though some important clinical concerns and safety points have remained.

Even though the positively charged quaternary amine dramatically lowers the lipophilicity of MNTX, indirect evidence has indicated that CNS effects can be elicited by MNTX. Abdominal pain has also been reported in 33-80% of the MNTX patients from two double-blind clinical trials, and cardiac-related adverse event occurred in less than 1% of patients in a long-term study. Moreover, although MNTX can be taken orally, three tablets of 150 mg are needed per day and it costs at least two thousand dollars for a 30-day course. Naloxegol and naldemedine, on the other hand, are less pricey, but still require approximately four hundred dollars for one month. Moreover, naloxegol is only prescribed to non-cancer pain patients. Also, the long-term use of naloxegol may induce many side effects, including abdominal pain, diarrhea, nausea, headache, and vomiting. What is more concerning is that naloxegol has been implicated in the possible elevated risk for a life-threatening arrhythmia. The newest PAMORA, naldemedine, also has the reported side effects of diarrhea and tearing of the stomach or intestine wall. Furthermore, both naloxegol and naldemedine are substrates of the CYP3A4 so that drug-drug interactions have been of concern. Therefore, it is still imperative to design and develop novel PAMORAs with more favorable tolerability profiles and to provide more choices for clinical applications.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

A series of non-peptide opioid receptor modulators are described herein. The modulators are novel chemical compounds that are used to treat neurological disorders related to opioid receptor functions. In some aspects, certain of the compounds cross the blood-brain barrier (BBB) and act on opioid receptors in the CNS. In other aspects, certain of the compounds do not cross, or do not significantly cross, the blood-brain barrier (BBB) and instead act on peripheral opioid receptors.

These novel compounds are potent and selective modulators (ligands) for opioid receptors and are used to treat neurological disorders and associated conditions as described herein. For example, depending on the structure of a compound, it targets principally the CNS MOR and is used to treat one or more of substance use disorders, e.g., opioid addiction, opioid overdose, alcoholism, as well as other neurological disorders related to opioid receptor functions; or it targets principally the PNS MOR and is used to prevent or treat opioid-induced constipation, irritable bowel movement disorders, etc.

It is an object of this invention to provide a compound having the general formula:

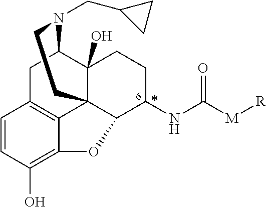

where
   R=

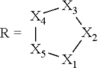

and is saturated, unsaturated, aromatic or heteroaromatic, where X1, X2, X3, X4 and X5 are independently C, N, O or S;
   M=a saturated or unsaturated, branched or unbranched, substituted or unsubstituted alkyl chain comprising from 0-10 carbon atoms;
and
   * indicates a chiral C with an α or β configuration,
and salts and stereoisomers thereof,
wherein -M-R is not

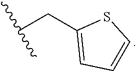 [SMILES: *Cc1cccs1]

In some aspects, M is 0, methyl or ethyl. In further aspects, R is

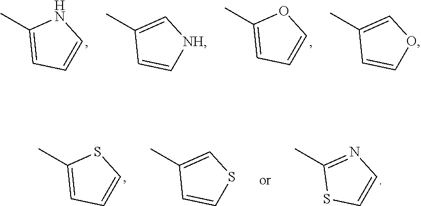

In additional aspects, M=0 and R is a 5-membered heterocyclic ring comprising one or more of S, N and NH. In yet further aspects, the chiral C has an a configuration and the compound is

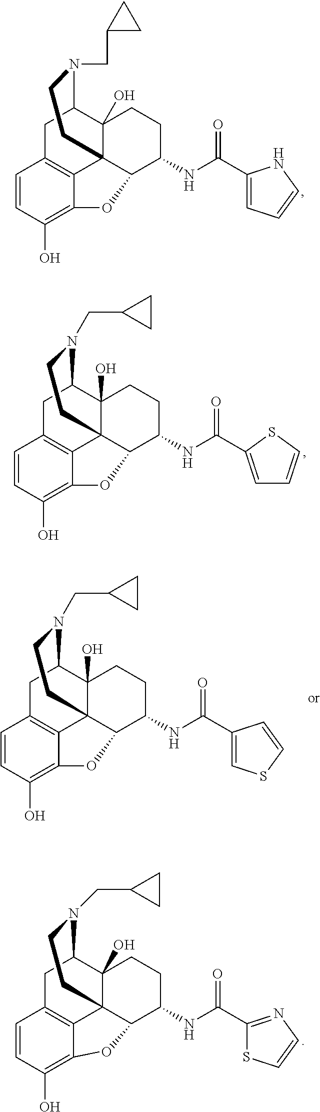

In some aspects, the compound is a hydrochloride salt.

In other aspects, M=0 or 1 and R is a 5-membered heterocyclic ring comprising N and NH.

In additional aspects, R is

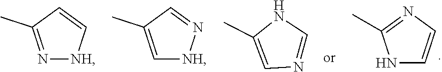

In yet further aspects, the chiral C has an a configuration and the compound is

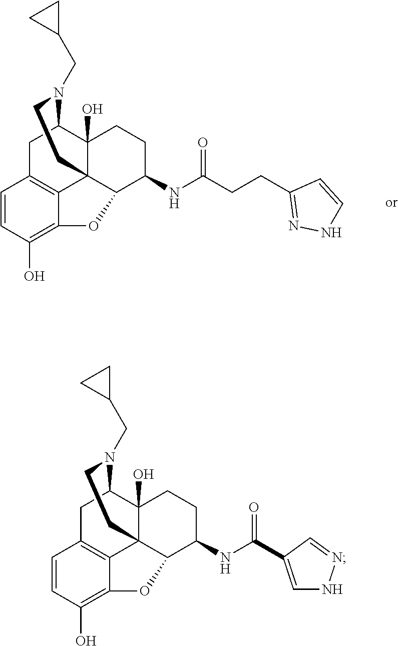

or
the chiral C has a β configuration the compound is

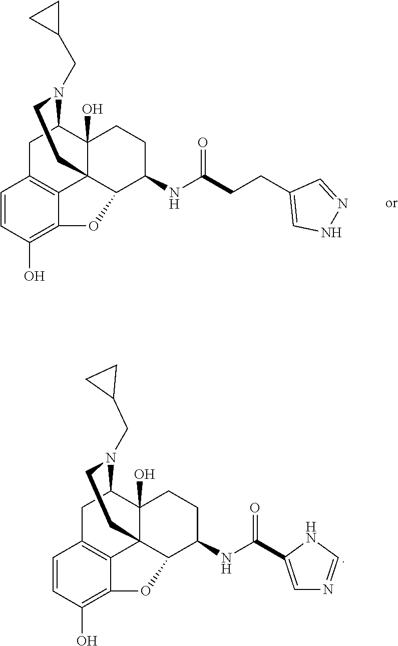

Also provided is a composition comprising at least one compound having the general formula:

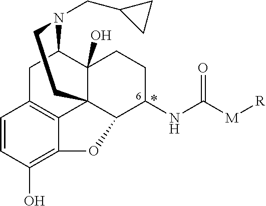

where
R=

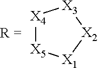

and is saturated, unsaturated, aromatic or heteroaromatic, where X1, X2, X3, X4 and X5 are independently C, N, O or S; M=a saturated or unsaturated, branched or unbranched, substituted or unsubstituted alkyl chain comprising from 0-10 carbon atoms; and * indicates a chiral C with an α or β configuration, and salts and stereoisomers thereof, wherein -M-R is not

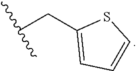 [SMILES: *Cc1cccs1]

and
a physiologically acceptable carrier. In some aspects, M is 0, methyl or ethyl. In further aspects, R is

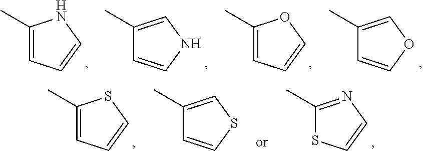

In additional aspects, M=0 and R is a 5-membered heterocyclic ring comprising one or both of S and NH. In yet further aspects, the chiral C has an a configuration and the compound is or

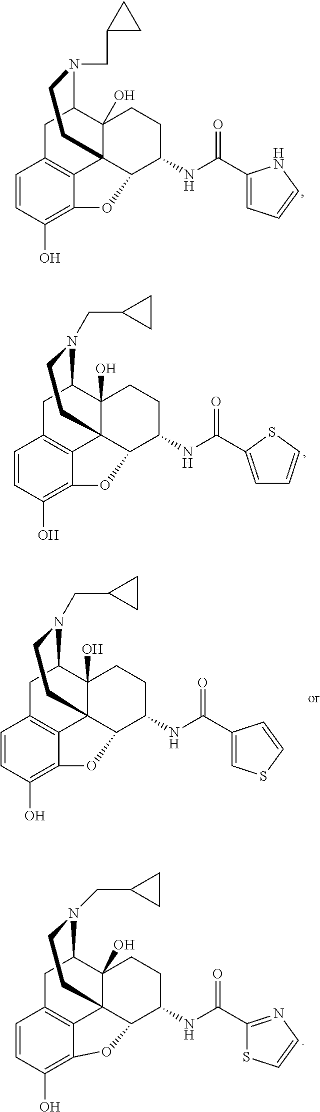

In some aspects, the compound is a hydrochloride salt.

In other aspects, M=0 or 1 and R is a 5-membered heterocyclic ring comprising N and NH.

In additional aspects, R is

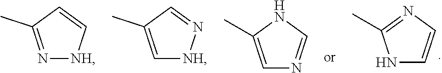

In yet further aspects, the chiral C has an α configuration and the compound is

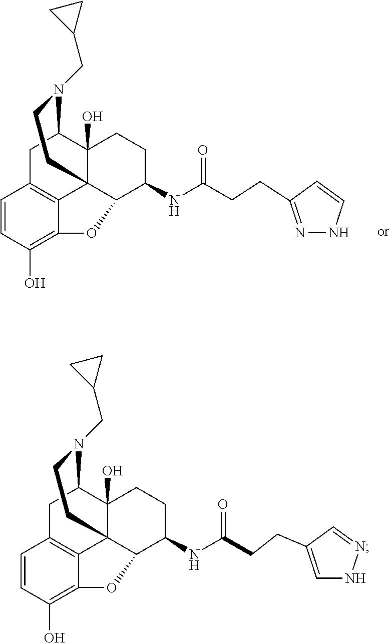

or
the chiral C has a β configuration the compound is

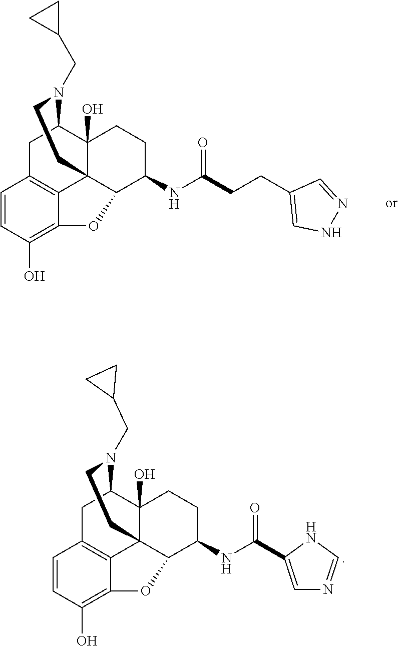

The disclosure further provides a method of preventing or treating addiction to an opioid in a subject in need thereof, comprising administering to the subject a therapeutically effective amount of at least one compound of formula

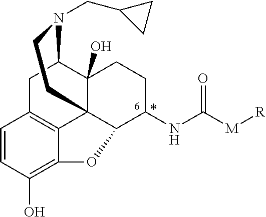

where R=

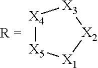

and is saturated, unsaturated, aromatic or heteroaromatic, where X1, X2, X3, X4 and X5 are independently C, N, O or S; M=a saturated or unsaturated, branched or unbranched, substituted or unsubstituted alkyl chain comprising from 0-10 carbon atoms; and * indicates a chiral C with an α or β configuration, and salts and stereoisomers thereof, wherein -M-R is not

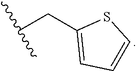 [SMILES: *Cc1cccs1]

In some aspects, M is 0, methyl or ethyl. In further aspects, R is

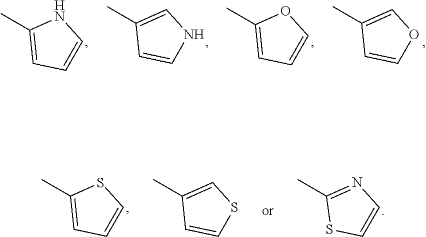

In additional aspects, M=0 and R is a 5-membered heterocyclic ring comprising one or more of S, N and NH. In yet further aspects, the chiral C has an α configuration and the compound is

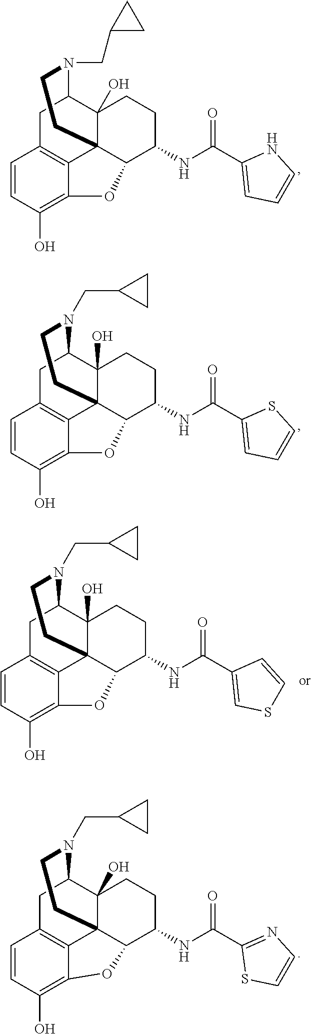

In some aspects, the compound is a hydrochloride salt.

In some aspects, the at least one compound is administered to the subject instead of the opioid. In further aspects, the at least one compound is

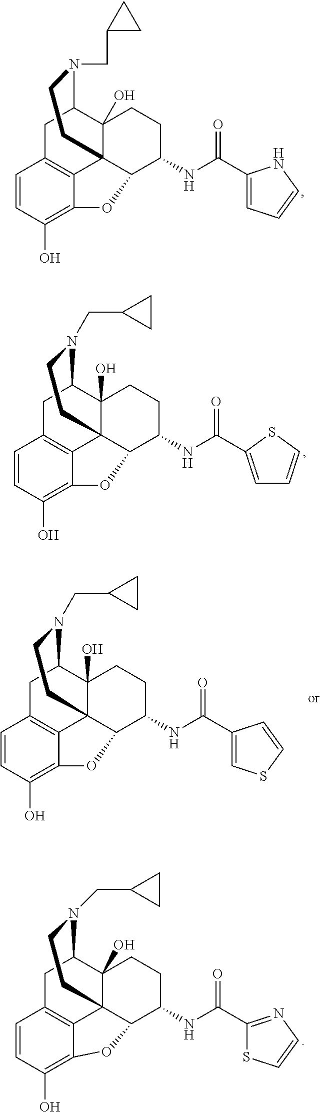

The disclosure also provides a method of treating opioid-induced constipation in a subject in need thereof, comprising administering to the subject a therapeutically efficient among of at least one compound of at least one compound having the general formula:

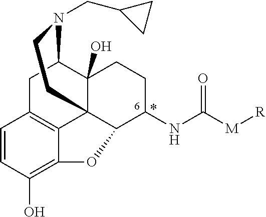

where
R=

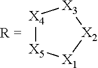

and is saturated, unsaturated, aromatic or heteroaromatic, where X1, X2, X3, X4 and X5 are independently C, N, O or S; M=a saturated or unsaturated, branched or unbranched, substituted or unsubstituted alkyl chain comprising from 0-10 carbon atoms; and * indicates a chiral C with an α or β configuration, and salts and stereoisomers thereof, wherein -M-R is not

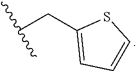 [SMILES: *Cc1cccs1]

In some aspects, M=0 or 1 and R is a 5-membered heterocyclic ring comprising N and NH. In further aspects, R is

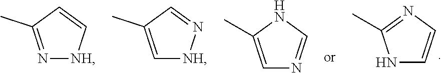

In yet further aspects, the chiral C has an α configuration and the compound is

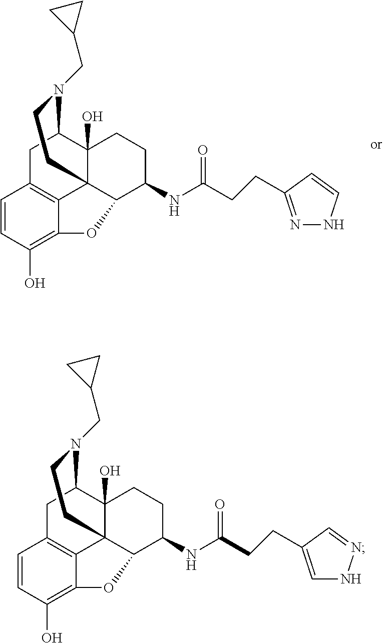

or
the chiral C has a β configuration the compound is

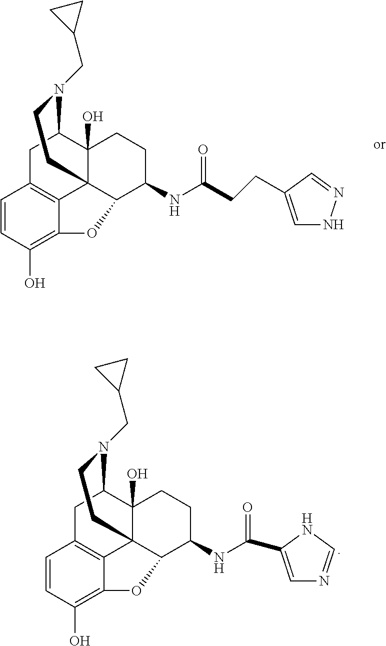

In other aspects, the at east one compound is administered in combination with an opioid. In further aspects, the at least one compound is administered orally. In additional aspects, a composition comprising the at least one compound and the opioid are provided. Thus, the at least one compound and the opioid are administered in a single dosage form.

### BRIEF DESCRIPTION OF THE DRAWINGS

FIGS. 1A and B. (A) Chemical structures of FDA-approved opioid ligands as opioid use disorder treatments. (B) Chemical structures of methylnaltrexone, naldemedine and naloxegol.

FIG. 2. Molecular design of NAP derivatives.

FIG. 3A-C. Water-water tail immersion assay results of (A) 6 α-analogs (n=6) as agonists at a single dose of 10 mg/kg s.c.; (B) 6β-analogs (n=6) as agonists at a single dose of 10 mg/kg s.c. and, (C) compounds as antagonists at a single dose of 10 mg/kg s.c. in the presence of morphine (10 mg/kg). Saline and morphine were used as the negative and positive controls respectively. Data are presented as mean values ±SD. *P<0.05, **P<0.01, and ***P<0.0005, ****P<0.0001, compared to 10 mg/kg morphine (s.c.).

FIG. 4A-C. In vivo withdrawal assays of compounds 25, 26 and 31 in morphine-pelleted mice (n=6), including (A) wet dog shakes, (B) jumps and (C) paw tremors. All compounds were administered s.c. *P<0.05, **P<0.01, and ***P<0.0005, ****P<0.0001, compared to 1 mg/kg naloxone (NLX; s.c.).

FIGS. 5A and B. (A) Tail-withdrawal assay. 10 mg/kg of each compound, vehicle or morphine was administered (s.c.) to a group of 6 mice. Compared with vehicle: #p<0.1, F (25, 130)=5.31; (B) Single-dose antagonism tail-withdrawal assay. 10 mg/kg of each compound, or vehicle, or 1 mg/kg naloxone (NLX) was given (s.c.) to a group of 6 mice 5 min prior to morphine injection (s.c.). Compared with vehicle+morphine group: **** p<0.0001, *** p<0.001, F (23, 119)=5.47. Mean % MPE value of each group is presented and the error bar represents SE.

FIGS. 6A and B. (A) Carmine red dye assay for compounds 2, 5, 17 and 19 administered subcutaneously; (B) Carmine red dye assay for compounds 2, 5, 17 and 19 administered orally. All testing compounds were given s.c. or via oral gavage at a dose of 10 mg/kg. Morphine was given subcutaneously at a dose of 10 mg/kg in all groups depicted above. Each group had at least 5 mice. The increased time requirement, compared with the respective vehicle group, was annotated above the bar for each group.

FIG. 7. Carmine red dye assay for compound 19, NAP and MNTX using 5 mg/kg morphine. All compounds were given via oral gavage at a dose of 10 mg/kg. Each group had at least 5 mice. The increased time requirement, compared with the vehicle group, was annotated above the bar for each group. Compared with 5 mg/kg morphine group: *p<0.1, F (14,70)=5.88).

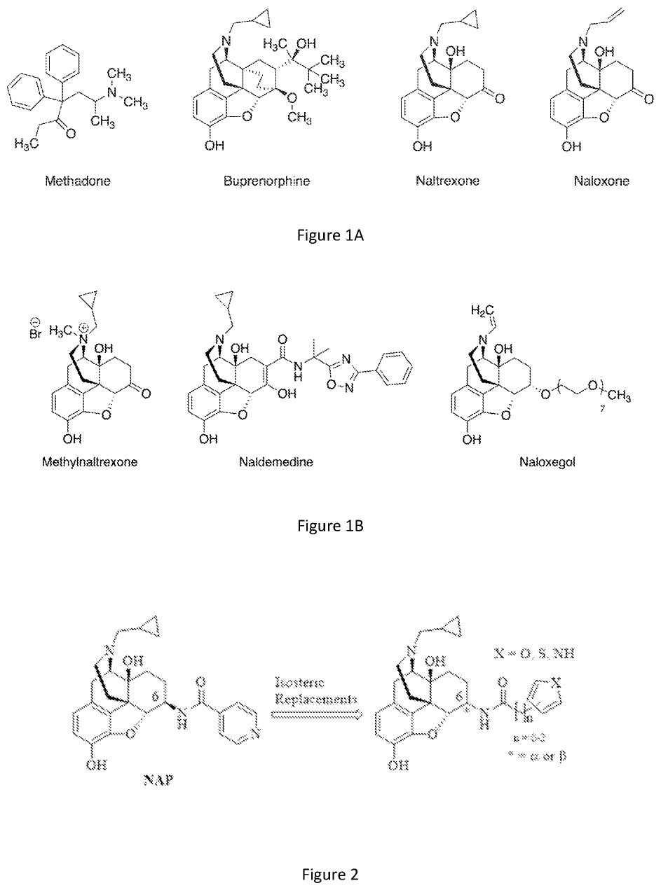

### DETAILED DESCRIPTION

Provided herein are compounds having the general formula:

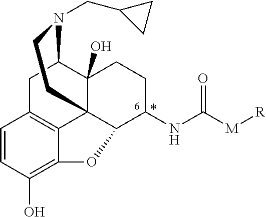

where R=

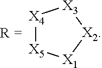

and is a saturated, unsaturated, aromatic or heteroaromatic 5-membered ring and is substituted or unsubstituted, where X1, X2, X3, X4 and X5 are independently C, N, O or S; M=a saturated or unsaturated, branched or unbranched (straight chain), substituted or unsubstituted alkyl group (e.g. an alkyl chain) comprising from 0-10 atoms;
* indicates a chiral C with an α or β configuration; and
-M-R is not

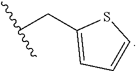 [SMILES: *Cc1cccs1]

Pharmaceutically acceptable salts and stereoisomers (enantiomers and diastereomers) of the compounds are also encompassed.

In some aspects, R is mono- or disubstituted, e.g., 3 or 4 atoms of X1, X2, X3, X4 and X5 are C and the remaining 1 or 2 atoms are, independently, heteroatoms such as N, S or O. If 2 heteroatoms are present in R, they may be the same or different, e.g., two N atoms, two S atoms or two O atoms, or a mixture of these may be present (N and O, N and S, O and S), as chemically possible. In other aspects, R is tri- or tetra-substituted, e.g. 1 or 2 atoms of X1, X2, X3, X4 and X5 are C and the remaining 3 or 4 atoms are independently, heteroatoms such as N, S or O. If 3 or 4 heteroatoms are present in R, they may be the same or different, e.g., two N atoms and one S atom; two N atoms, one S atom and one O atom; and so on for all such combinations, as chemically possible.

The chiral carbon at the 6-position (*) is in either the a or β configuration.

M is a saturated or unsaturated, substituted or unsubstituted straight-chain or branched, alkyl group (e.g. an alkyl chain) of from 0-10 atoms, which are C atoms unless M is substituted. If M=0, then M is absent.

For the R group, the connection point to M (or to the carbon of the amide group if M is absent) is at any one of the X1, X2, X3, X4, or X5 atoms. The ring system, R, is generally saturated, unsaturated, aromatic or heteroaromatic and X1 to X5 are independently carbon (C), or a heteroatom such as nitrogen (N), oxygen (O), or sulfur (S) atom, generally with or without one or two hydrogen atom(s) attached thereto, as chemically possible. For example, those of skill in the art will recognize that if X3 is C in a saturated ring, then the C group will have two covalently attached H atoms (X3=—CH₂) whereas if the ring is not saturated and the X3 group is attached to a double bond of the ring, then the C group is —CH (C3=—CH), or if X3 is located between two double bonds, then no H is attached and C3=C, and so on for each atom of the ring.

In some aspects, R is pyrrole, furan, or thiophene.

As used herein, “alkyl” by itself or as part of another substituent refers to a saturated or unsaturated, branched, or straight-chain, substituted or unsubstituted monovalent hydrocarbon radical derived by removal of one hydrogen atom from a single carbon atom of a parent alkane, alkene, or alkyne. Examples of suitable alkyl groups include, but are not limited to, methyl; ethyl, such as ethanyl, ethenyl, and ethynyl; propyl such as propenyl, propyl-1-en-2-yl, prop-2-en-1-yl (allyl), prop-1-yl-1-yl, prop-2-yn-1-yl, prop-1-yl, and the like; butyl such as butanyl, but-1-en-1-yl, but-1-en-yl, but-2-en-2-yl, buta-1, 3-dien-1-yl, but-1-yn-3-yl, but-3-yn-1-yl; pentyl and its isomeric forms, and the like for groups with 6, 7, 8, 9, or 10 atoms.

Unless otherwise indicated, the term “alkyl” means a group having any degree of saturation or degree of unsaturation, i.e., a group having only single carbon-carbon bonds, a group having at least one, two, three double carbon-carbon bonds, etc. Groups having a mixture of single, double and triple carbon-carbon bonds are encompassed. When a certain degree of saturation is intended, the terms “alkenyl,” “alkenyl,” and “alkynyl” are used. In certain embodiments, M contains from 0 to 10 atoms, e.g., from 0 (M is absent) to about 1, 2, 3, 4, 5, 6, 7, 8, 9 or 10 atoms, generally C atoms. However, if M is substituted, one or more of the atoms may be, e.g., N, S, O, C═O, etc., with H atoms attached to the heteroatom as required or permitted chemically, such as according to the pH of the medium in which the compound is present.

As used herein, “substituted” generally refers to i) the replacement of a carbon atom by a non-carbon atom e.g., in the main chain of a carbon chain or carbon ring structure; or ii) the replacement of an H atom that is attached to a C of a carbon chain or carbon ring by a non-H atom, or a combination of i) and ii).

In some aspects, such as those described in Example 1 and Example 3, M=0, 1 or 2 (i.e., M is absent, methyl or ethyl) and the R group is

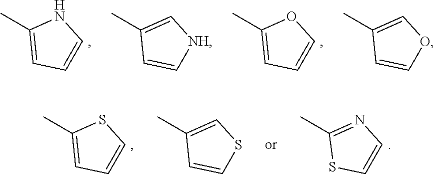

In some aspects, such as those described in Example 1 and Example 3, the M-R group is

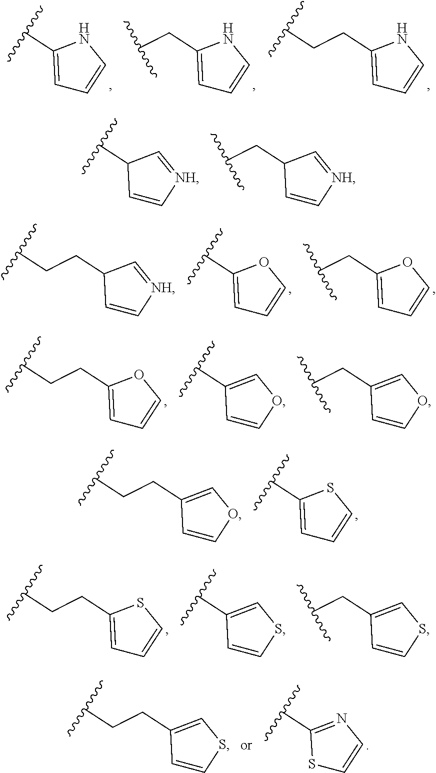

In some aspects, such as those described in Example 2, M=0, 1 or 2 and R is

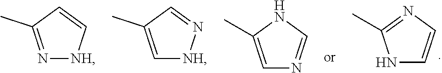

In some aspects, such as those described in Example 2, Table 9, the M-R group is

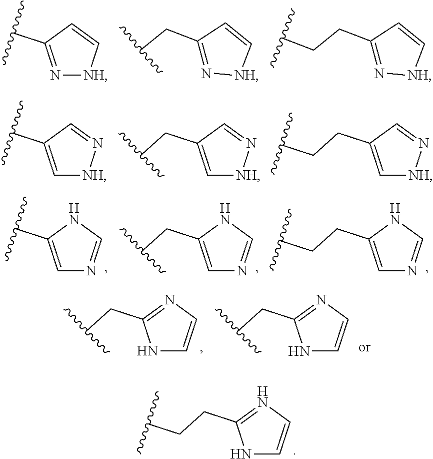

In preferred aspects of the disclosure, the compounds that are used as CNS antagonists are compounds depicted in Example 1 as HCl salts 1, 25 and 31 and the compound depicted in Example 3 as an HCl salt, all of which have the chiral C in the a configuration:
17-Cyclopropylmethyl-3,14β-dihydroxy-4,5α-epoxy-6a-[(2′-pyrrolyl)carboxamido]morphinan (NAY) (HCl salt is compound 1 of Example 1)

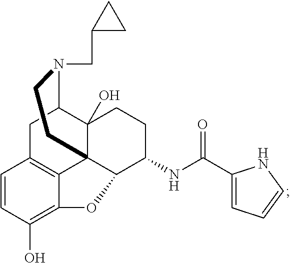

17-Cyclopropylmethyl-3,14β-dihydroxy-4,5α-epoxy-6α-(2′-thienylcarboxamido)morphinan (NAT) (HCl salt is compound 25 of Example 1)

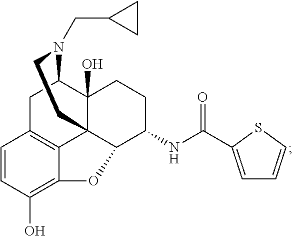

17-Cyclopropylmethyl-3,14β-dihydroxy-4,5α-epoxy-6α-(3′-thienylcarboxamido)morphinan (NAH) (HCl salt is compound 31 of Example 1)

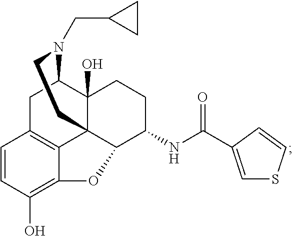

and/or
17-Cyclopropylmethyl-3,14β-dihydroxy-4,5α-epoxy-6α-(2′-thiazolylcarboxamido)morphinan (NTZ) (the compound whose synthesis is described in Example 3 as an HCl salt)

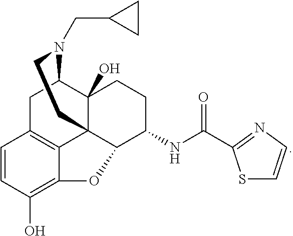

Characteristics of these four compounds are set forth in Tables A and B, where the is HCl salts are depicted and described.

TABLE A
Characteristics of compounds 31 and 25 of Example 1.

| Code in Patent Application | | | Example 1 compound 31 | Example 1 compound 25 |
|---|---|---|---|---|
| Compound Name | | | NAH | NAT |
| Structure Physical properties | | | 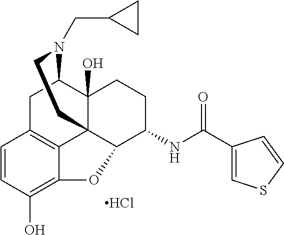 | 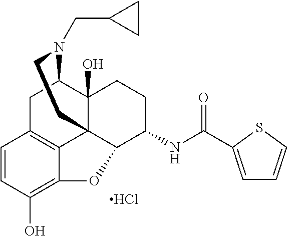 |
| | | | Code: VZMN234 | Code: VZMN231 |
| | | | Name: AK-I-89 | Name: AK-I-65 |
| | | | MW: 452.57 | MW: 452.57 |
| | | | FW: 489.03 | FW: 489.03 |
| | | | Solubility: DMSO, H₂O | Solubility: DMSO, H₂O |
| NIDA screening code | | | 34318 | 34189 |
| Binding | MOR | | 0.38 ± 0.04 | 0.37 ± 0.01 |
| Affinity | DOR | | 10.71 ± 3.56 | 24.28 ± 5.08 |
| Ki (nM) ± | KOR | | 2.80 ± 0.12 | 1.59 ± 0.17 |
| SEM | | | | |
| Function | MOR | Potency | 1.07 ± 0.22 | 0.49 ± 0.09 |
| potency | | EC₅₀, nM | | |
| and | | Efficacy | 30.66 ± 4.44 | 21.81 ± 6.17 |
| efficacy | | (% max of | | |
| ³⁵S- | | DAMGO) | | |
| GTP[γS]- | | | | |
| Binding | | | | |
| | DOR | Potency | 38.8 ± 5.83 | 59.41 ± 6.71 |
| | | EC₅₀, nM | | |
| | | Efficacy | 58.5 ± 3.13 | 48.58 ± 3.41 |
| | | (% max of | | |
| | | SNC80) | | |
| | KOR | Potency | 16.3 ± 1.73 | 1.72 ± 0.24 |
| | | EC₅₀, nM | | |
| | | Efficacy | 47.8 ± 0.85 | 50.56 ± 0.95 |
| | | (% max of | | |
| | | U50, 488H) | | |

| Calcium flux (on MOR-CHO | IC₅₀ = 28.90 ± 5.34 nM | | IC₅₀,_{NAT} = 26.7 ± 8.9 nM | |
|---|---|---|---|---|
| cells), acting as antagonists | nM (against DAMGO) | | (against DAMGO) | |
| Tail flick assay (mice, single | The percentage maximum possible effect (% MPE) | | The percentage maximum possible | |
| dose) | of NAH (10 mg/kg) was 17.2 ± 9.8 compared to | | effect (% MPE) of NAT (10 mg/kg) was 15.2 ± | |
| | 95.5 ± 4.5% for morphine (10 mg/kg). | | 8.8% compared to 96.5 ± 4.5% for morphine (10 | |
| | | | mg/kg). | |
| Tail flick assay (mice, dose | The AD₅₀ of NAH was determined as 1.51 (1.08- | | The AD_{₅₀}of NAT was determined as 0.42 (0.21- | |
| response) | 2.09) mg/kg (95% CL) | | 0.82) mg/kg (95% CL) | |
| Withdrawal study (morphine- | The number of wet-dog shakes, escape jumps, and | | At 1 mg/kg dose of NBF produced NO wet-dog | |
| pelleted mice) | paw tremors for NAH at a high dose of 5 and 10 | | shakes, escape jumps, and paw tremors compared to | |
| | mg/kg is somehow comparable or lower to | | 1 mg/kg of naloxone. The number of wet-dog shakes, | |
| | naloxone at 1 mg/kg. A 1 mg/kg dose of NAH | | escape jumps, and paw tremors for NAT at a high dose | |
| | produced significantly fewer wet-dog shakes, | | of 5 mg/kg is somehow compared to naloxone at 1 mg/kg. | |
| | escape jumps, and paw tremors than 1 mg/kg | | Even higher dose was not tested. | |
| | naloxone. | | | |
| Self-administration Studies | No significant abuse liability | | No significant abuse liability | |
| Abuse liability | | | | |
| Self-administration Studies | reduce heroin choice and increase food choice in a | | reduce heroin choice and increase food choice in a | |
| Food vs Drug | self-administration rat model | | self-administration rat model | |
| side effect profiling | No significant binding up to 10 μM except: | | No significant binding up to 10 uM except: | |
| Serotonin receptor, dopamine | h5-HT1b (98% at 10 uM) | | h5-HT2b (78% at 10 uM) | |
| receptor, biogenic amine | hM1 (62% at 10 uM) | | hNOP (60% at 10 uM), Ki 6.4 uM | |
| transporters | hM2 (82% at 10 uM) | | hM1 (53% at 10 uM), Ki 8.4 μM | |
| | hM4 (65% at 10 uM) | | hM2 (81% at 10 uM), Ki 1.9 μM | |
| | hNK1 (80% at 10 uM) | | hM4 (61% at 10 uM), Ki 4.9 μM | |
| | hERG channel (58% at 10 uM) | | hNK3 (56% at 10 uM), Ki 3.9 μM | |
| | | | Na channel (55% at 10 uM), Ki 9.9 μM | |
| hERG channel testing | 1.3 μM (Eurofins) | | 2.5 μM (Eurofins) | |
| AMES testing | No toxicity shown | | No toxicity shown | |
| CYP450 enzyme testing | Only moderate at CYP2D6 and CYP2C19 | | Only CYP2C19 inhibition (~3 uM) | |
| | inhibition | | | |
| Protein Binding (plasma) | Human, 60 | Rat, 56 | Human, 37 | Rat, 64 |
| Permeability (Caco-2) | 4.0 | | 2.2 | |
| B-A/A-B | | | | |
| Metabolic Stability (half-life) | Human, >60 min | Rat, 44 min | Human, >60 min | Rat, >60 min |

| | | |
|---|---|---|
| Glucuronidation/Oxidation? | | |
| In vivo Bioavailability Foral | 15% | 19% |

TABLE B
Characteristics of compound 1 of Example 1 and “NTZ” of Example 3.

| Code in Patent Application | | | Example 1 compound 1 | Example 3 |
|---|---|---|---|---|
| Compound Name | | | NAY | NTZ |
| Structure Physical properties | | | 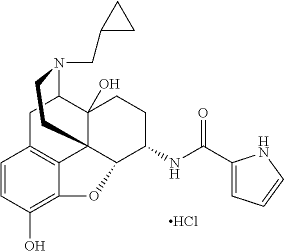 | 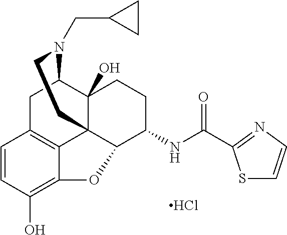 |
| | | | Code: VZMN249 | Code: VZMN283 |
| | | | Name: YZ-II-82 | Name: BH-VI-119 |
| | | | MW: 435.22 | MW: 453.56 |
| | | | FW: 471.98 | FW: 490.02 |
| | | | Solu: DMSO, H₂O | Solubility: DMSO, H₂O |
| NIDA screening code | | | 34226 | 34319 |
| Binding | MOR | | 0.32 ± 0.03 | 0.25 ± 0.03 |
| Affinity | DOR | | 24.66 ± 2.22 | 41.07 ± 3.58 |
| Ki (nM) ± | KOR | | 1.58 ± 0.16 | 1.30 ± 0.12 |
| SEM | | | | |
| Function | MOR | Potency | 3.39 ± 1.29 | 2.16 ± 0.38 |
| potency | | EC₅₀, nM | | |
| and | | Efficacy | 12.11 ± 2.64 | |
| efficacy | | (% max of | | |
| ³⁵S- | | DAMGO) | | 11.29 ± 1.30 |
| GTP[γS]- | | | | |
| Binding | | | | |
| | DOR | Potency | 26.88 ± 3.23 | 23.56 ± 4.29 |
| | | EC₅₀, nM | | |
| | | Efficacy | 50.94 ± 5.26 | 46.51 ± 2.17 |
| | | (% max of | | |
| | | SNC80) | | |
| | KOR | Potency | 14.83 ± 0.99 | 3.83 ± 0.62 |
| | | EC₅₀, nM | | |
| | | Efficacy | 43.37 ± 1.36 | 23.51 ± 2.03 |
| | | (% max of | | |
| | | U50, 488H) | | |

| Calcium flux (on MOR-CHO | IC_{50,NAY} = 4.65 ± 0.96 nM (against DAMGO) | | | |
|---|---|---|---|---|
| cells), acting as antagonists | | | | |
| Tail flick assay (mice, single | No anti-nociceptive effect up to 100 mg/kg. | | No anti-nociceptive effect at 10 mg/kg, but | |
| dose) | | | significantly antagonized morphine's antinociceptive | |
| | | | effect at 10 mg/kg | |
| Tail flick assay (mice, dose | The AD₅₀ of NAY was determined as 4.71 (1.61- | | The AD₅₀ of VZMN283 was determined as 0.043 | |
| response) | 13.81) mg/kg (95% CL) | | (0.007-0.25) mg/kg (95% CL) | |
| Withdrawal study (morphine- | At 1 mg/kg dose of NAY produced NO wet-dog | | No significant precipitation of withdrawal syndromes | |
| pelleted mice) | shakes, escape jumps, and paw tremors compared | | up to 0.1 mg/kg. Higher dose at 1 mg/kg showed | |
| | to 1 mg/kg of naloxone. The number of wet-dog | | fewer symptoms than naloxone at the same dose. | |
| | shakes, escape jumps, and paw tremors for NAT | | | |
| | at a high dose of 5, 10, 20 mg/kg is still much | | | |
| | lower compared to naloxone at 1 mg/kg. | | | |
| | Even higher dose was not tested. | | | |
| Self-administration Studies | No significant abuse liability | | No significant abuse liability | |
| Abuse liability | | | | |
| Self-administration Studies | reduce heroin choice and increase food choice | | reduce heroin choice and increase food choice in a | |
| Food vs Drug | in a self-administration rat model | | self-administration rat model | |
| side effect profiling | No significant binding up | | No significant binding up | |
| Serotonin receptor, dopamine | to 10 μM except: | | to 10 μM except: | |
| receptor, biogenic amine | h5-HT1b (85% at 10 uM) Ki 482.2 nM | | hM1 (53% at 10 uM) | |
| transporters | hNOP (59% at 10 uM), Ki 1.44 μM | | hM2 (88% at 10 uM) | |
| | hM2 (58% at 10 uM), Ki 7.28 μM | | hM4 (67% at 10 uM) | |
| | Potassium channel hERG (52% at 10 uM), | | | |
| | Ki 8.60 μM hNK1, (89% at 10 uM) hα1D, | | | |
| | (84% at 10 uM) Ki 770.5 nM | | | |
| hERG channel testing | 2.6 μM (Eurofins)/12.5 μM (NIH) | | 7.4 μM (Eurofins) | |
| AMES testing | No toxicity shown | | No toxicity shown | |
| CYP450 enzyme testing | Only moderate at CYP2D6 and CYP2C19 | | Only moderate at CYP2C19 inhibition and | |
| | inhibition | | potent at CYP2D6 inhibition | |
| Protein Binding (plasma) | Human, 57 | Rat, 57 | Human, 44 | Rat, 60 |
| Permeability (Caco-2) | 10 | | 2.5 | |
| B-A/A-B | | | | |
| Metabolic Stability (half-life) | Human, >60 min | Rat, >60 min | Human, >60 min | Rat, >60 min |

| | |
|---|---|
| Glucuronidation/Oxidation? | |
| In vivo Bioavailability Foral | 59% |

“Pharmaceutically acceptable salts” of the compounds refers to the relatively non-toxic, inorganic and organic acid addition salts and base addition salts of compounds of the present disclosure. In some aspects, these salts are prepared in situ during the final isolation and purification of the compounds. In some aspects, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like. In some aspects, the salt is an HCl (hydrochloride) salt.

In some aspects, diseases and conditions associated with opioid receptors are prevented or treated. Thus, methods of agonizing, partially agonizing or antagonizing one or more opioid receptors to treat such diseases and conditions is provided. This is generally accomplished by administering one or more compounds as described herein as agonists (activators), partial agonists or antagonists (inhibitors) of at least one opioid receptor, i.e., at least one of the Mu-opioid receptor (MOR), the Kappa-opioid receptor (KOR) or the Delta-opioid receptor (DOR).

In particular, the MOR is targeted by the compounds disclosed herein, and further, the targeting is by compounds that are predominantly:
i) central nervous system (CNS) acting MOR antagonists, especially those of Example 1); or
ii) peripheral nervous system (PNS) MOR antagonists, especially those of Example 2.

CNS acting MOR antagonists are typically used to treat e.g., opioid addiction, and neurological disorders whereas peripherally acting (PA or PNS) MOR antagonists thwart MOR receptor activation in the gastrointestinal (GI) tract, thereby preventing or treating opioid-induced constipation.

Accordingly, methods of antagonizing CNS MOR are encompassed and comprise contacting the CNS MOR with at least one compound described in Example 1 or 3, where the R group of the present compounds is

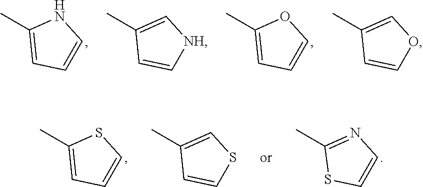

Alternatively, methods of antagonizing PNS MOR are encompassed and comprise contacting the PA MOR with at least one compound described in Example 2, where the R group of the compounds is

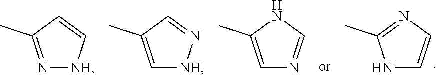

All these methods may be carried out in vitro (e.g., in a laboratory setting using cell culture, isolated opioid receptors, etc.) or in vivo, also in a laboratory setting (e.g., in an animal model) or in a subject or patient in need thereof for the purpose of medical treatment, as described fully below. All such methods are encompassed herein.

The compounds described herein are advantageously non-toxic (are of low toxicity) to mammals when administered in vivo.

The compounds described herein advantageously display a high level of binding affinity and/or selectivity and/or potency and/or efficacy for a particular opioid receptor, either that of the CNS or that of the PNS. Diseases and conditions that can be treated using the pharmacological agents described herein include but are not limited to those associated with opioid receptors. In some aspects, the compounds (typically those disclosed in Examples 1 and 3 which target the CNS MOR) are used for the treatment of substance use and/or abuse disorders, e.g. opioid addiction and overdose, alcoholism, as well as other neurological disorders related to opioid receptor functions, whereas those described in Example 2 are used to treat e.g. opioid induced constipation, irritable bowel movement disorders, etc. by targeting PNS MOR. The compounds are thus useful to combat drug (opioid) abuse (including opioid overdose) and/or addiction, opioid use and/or addiction in those being treated for pain; constipation in those being treated for pain by taking opioids; for the treatment of pain (e.g., taken instead of an opioid as an opioid substitute), or taken with an opioid, etc.

With respect to substance use and/or abuse disorders such as opioid addiction, the compounds may be used as non- or less addictive substitutes for those who are at risk of becoming addicted, or who are already addicted due to opioid use and are trying to stop or decrease opioid intake, or who experience an opioid overdose, either voluntarily or in a setting in which they are required to do so. The compounds may be used as a safer alternative to (instead of) opiates to prevent addiction in the first place, e.g. for the treatment of pain (see below). The addiction that is treated may be due to the overuse of prescription opiates or to addiction resulting from the purely recreational (usually illegal) use of opiates.

With respect to treating opioid addiction and/or preventing relapse, compounds: 17-cyclopropylmethyl-3,14β-dihydroxy-4,5α-epoxy-6a-[(2′-pyrrolyl)carboxamido]morphinan (NAY) (HCl salt is compound 1 of Example 1); 17-cyclopropylmethyl-3,14β-dihydroxy-4,5α-epoxy-6α-(2′-thienylcarboxamido)morphinan (NAT) (HCl salt is compound 25 of Example 1); 17-cyclopropylmethyl-3,14β-dihydroxy-4,5α-epoxy-6α-(3′-thienylcarboxamido)morphinan (NAH) (HCl salt is compound 31 of Example 1); and 17-cyclopropylmethyl-3,14β-dihydroxy-4,5α-epoxy-6α-(2′-thiazolylcarboxamido)morphinan (NTZ) (the compound whose synthesis is described in Example 3 as an HCl salt); are generally used.

With respect to neurological disorders or conditions which are prevented or treated, such diseases/disorders include but are not limited to: neurodegeneration of any type e.g. those caused by bacterial or viral infections (e.g. neuro-AIDS); Alzheimer's disease; Parkinson's disease; dementia; depression and/or anxiety; aberrant behavioral changes; a gradual decline in cognitive function, including trouble with concentration, memory, and attention; progressive slowing of motor function and loss of dexterity and coordination; central nervous system (CNS) lymphomas; neurological symptoms of herpes virus infections; nerve damage and pain; encephalitis; traumatic head injury; sports related head injury; etc.

In some embodiments, the neurological diseases or conditions are associated with individuals whose immune systems are compromised, e.g. as a result of medical treatments (e.g. chemotherapy, etc.), as a result of disease such as human immunodeficiency virus infection/acquired immunodeficiency syndrome (HIV/AIDS), as a result of one or more genetic disorders or mutations, as a result of environmental insult or challenges (e.g. poor nutrition, excessive stress, pollutants, etc.), or due to advancing age.

With respect to neurological disorders or conditions that are prevented or treated, compounds: 17-cyclopropylmethyl-3,14β-dihydroxy-4,5α-epoxy-6a-[(2′-pyrrolyl)carboxamido]morphinan (NAY) (HCl salt is compound 1 of Example 1); 17-cyclopropylmethyl-3,14β-dihydroxy-4,5α-epoxy-6α-(2′-thienylcarboxamido)morphinan (NAT) (HCl salt is compound 25 of Example 1); 17-cyclopropylmethyl-3,14B-dihydroxy-4,5α-epoxy-6α-(3′-thienylcarboxamido)morphinan (NAH) (HCl salt is compound 31 of Example 1); and 17-cyclopropylmethyl-3,14β-dihydroxy-4,5α-epoxy-6α-(2′-thiazolylcarboxamido)morphinan (NTZ) (the compound whose synthesis is described in Example 3 as an HCl salt); are generally used.

Alternatively, when opioids are used to treat acute pain, or especially when they are used to treat chronic pain, uncomfortable and even severe constipation can result, adding significantly to the well-being of the patient. The compounds disclosed herein (typically those disclosed in Example 2) are given to prevent or treat such opioid induced constipation and are typically, but not always, administered with an opioid. Exemplary opioids with which the compounds are administered include but are not limited to: oxycodone (OxyContin), hydrocodone (Vicodin), morphine, methadone and the artificial opioid fentanyl. Patient populations for whom opiates are frequently prescribed and who are at risk of developing constipation include but are not limited to: those with terminal illnesses or conditions such as cancer or severe injuries; those with short term pain such as subjects who have had surgery; those will long-term but non-terminal illnesses such as those with pain-inducing progressive skeletal or nerve disorders, etc.

With respect to treating opioid-induced constipation in particular, the compounds depicted in Example 2, are generally used, for example: compounds 2, 5, 17 and 19, and, in particular, compounds 2 and 19 of Example 2.

With respect to irritable bowel movement disorders that are prevented or treated generally using the compounds of Example 2, examples include irritable bowel syndrome (IBS, e.g., characterized by mostly diarrhea and abdominal discomfort (IBS-D; mostly constipation and abdominal discomfort (IBS-C); alternating loose stools and constipation with abdominal discomfort (IBS-mixed); and undefined subtype (IBS-U) in which symptoms vary); inflammatory bowel disease (IBD) characterized by destructive inflammation and permanent harm to the intestines; etc.

With respect to irritable bowel movement disorders that are prevented or treated compounds 2, 5, 17 and 19 in Example 2 are generally used.

In general, the individuals who are treated as described herein already exhibit gross, observable and usually measurable symptoms of such as neurological damage, pain and/or constipation. In such instances, the methods may include a step of identifying individuals suitable for receiving treatment using known examination techniques and other tests such as blood tests, viral and/or bacterial culture, self-reported discomfort, etc. Patients identified as positive for symptoms are deemed candidates for treatment.

In some aspects, the individuals who can benefit from receiving the agents described herein do not yet display overt symptoms of the disease or condition to be treated but are known to be at risk of developing the disease or condition. For example, a person would be a candidate for prophylactic (or simultaneous) treatment even prior to the emergence of overt symptoms might include one who is: known to abuse or be addicted to opiates, suffering from alcoholism, is going to undergo chemotherapy other immuno-compromising or painful procedure, a person known to have any other disease or condition (e.g. a genetic predisposition) toward developing a compromised immune system and/or neurological damage or neurodegeneration, or a person who is undergoing treatment with opiates (e.g. for pain management) and is likely to develop constipation, etc. In such cases, at least one symptom of the disease or condition may be prevented or at least lessened (ameliorated). In some cases, complete prevention or eradication of symptoms is achieved, i.e., the patient never develops any symptoms or is cured of all symptoms.

The individuals who are treated using the agents and methods of the invention are generally mammals, and are usually, but not always, humans. However, veterinary applications of this technology are also contemplated. The individuals (subjects, patients, etc.) that are treated may be adults or juveniles (e.g., children).

The present invention also provides compositions for use in preventing and/or treating dysfunction and/or bodily dysfunction caused using opioids or various diseases or conditions described herein which are associated with opioid receptors. The compositions include one or more substantially purified compounds as described herein, and a pharmacologically (physiologically) suitable (compatible) carrier. The preparation of such compositions is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. If it is desired to administer an oral form of the composition, various thickeners, flavorings, diluents, emulsifiers, dispersing aids or binders and the like may be added. The composition of the present invention may contain any such additional ingredients to provide the composition in a form suitable for administration. Other suitable medications may also be administered together with the compounds, either separately in different preparations, or together in the same preparation. The final amount of compound in the formulations may vary. However, in general, the amount in the formulations will be from about 1-99%.

The compositions may be pills, capsules, etc. designed for long-term or slow (extended) release. The compounds may be incorporated into a patch or implantable insert for long term release.

The compositions (preparations) of the present invention may be administered by any of the many suitable means which are well known to those of skill in the art, including but not limited to by injection (e.g. intravenous, intraperitoneal, intramuscular, subcutaneous, intra-aural, intraarticular, into the spinal column, intracranial, and the like); by inhalation; orally; intravaginally, intranasally, topically (by absorption through epithelial or mucocutaneous linings e.g., nasal, oral, vaginal, rectal, gastrointestinal mucosa, and the like); as eye drops; via sprays, by a patch that is attachable to the skin of a patient; or via an implantable delivery device; etc. In preferred embodiments, the mode of administration is by injection. In addition, the compositions may be administered in conjunction with other treatment modalities such as substances that boost the immune system, various chemotherapeutic agents, antibiotic agents, and the like.

As described in Example 2 below, in some aspects such as treating constipation, oral administration is generally preferred.

The methods generally involve administering to a subject (patient) in need thereof a therapeutically effective amount of one or more of the compounds described herein. The exact dosage that will be administered, as well as the mode and frequency of administration, will vary from subject to subject, with guidance being provided by clinical trials data.

However, in general, it is believed that a dose in the range of from about from 1 mg to about 600 mg per day, more specifically from about 10 mg to about 100 mg range, is administered, for example, about 1, 2, 3, or 4 times per day, unless administered intravenously in which case the dosage is adjusted accordingly to achieve suitable biologically active and effective levels of the agent(s) in the subject's bloodstream.

In other aspects, methods of agonizing, partially agonizing or antagonizing one or more opioid receptors is provided. The methods comprise contacting at least one opioid receptor (typically MOR, but alternatively KOR and/or DOR) with a compound of the disclosure. These methods may be carried out in vitro or in vivo for any purpose.

In further aspects, methods of preventing or treating constipation caused by opioids are provided. The methods involve administering a compound as described in Example 2 in combination with the opioid. The compound described in Example 2 may be taken together with an opioid, either at the same time, or at alternate times but within the time frame of opioid activity (e.g., before or after opioid administration). Alternatively, compositions comprising at least one opioid and at least one compound disclosed in Example 2 in a single composition, i.e., in a pill or liquid formulation so that the two agents are both administered in a single dose.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Example 1. Design, Synthesis, and Biological Evaluation of NAP Isosteres: A Switch from Peripheral to Central Nervous System Acting Mu-Opioid Receptor Antagonists

Using the highly selective MOR antagonist 17-cyclopropylmethyl-3,14-dihydroxy-4,5α-epoxy-6β-[(4′-pyridyl)carboxamido]morphinan (NAP) as a lead, the concept of isosteric replacement was employed to replace the pyridine ring in the address moiety of NAP with its isosteric counterparts: pyrrole, furan, and thiophene, even though there was a possibility of loss of hydrogen bonding interaction with Lys303 residue resulting in lowered selectivity over the KOR and DOR. Additionally, compounds possessing an acetamido or n-propanamido linker between the “message” and “address” moieties were designed to help probe the influence of distance and flexibility of the aromatic ring on parameters such as affinity and selectivity. The linker substitution position was also varied to study the role of orientation of the aromatic ring on biological activities. Finally, although NAP has a defined stereocenter (β) at C6, the structure-activity relationship of the C6 stereochemistry has not been conclusive. For example, while NAP and NBF carry β configuration, two other compounds, NAQ and NAN carry a configuration at C6. Since NAP, NBF, NAQ and NAN were identified as MOR-selective ligands, it seemed that the C6 stereochemistry had little effect on the interaction of the address portion of the molecule with Lys303. Therefore, we decided to synthesize compounds with both configurations at C6 in this study. Thus, a total of 36 compounds were designed with the following characteristics: (1) the stereochemistry at C(6) may be either a or P; (2) the linker between the epoxymorphinan skeleton and C(6) side chain may either be carboxamido, acetamido or n-propanamido; (3) the linker could be attached to either the 2′ or 3′ position of the aromatic ring (FIG. 2). Of these, two compounds, 27 and 28 have been previously studied as potential alcohol-cessation agents. Thus total 34 new compounds were synthesized. Additionally, the physicochemical parameters were predicted for all the newly designed ligands by using ACD/Percepta (v2020.2.0) (Advanced Chemistry Development, Inc., Toronto, ON, Canada, www.acdlabs.com, 2021). ACD Percepta. and it was found that the different parameters fell in the following range for all derivatives designed: c Log P=1.71-3.04, c Log D=0.94-2.40 and pKa=7-7.5 (not shown). These values cover a range around the ideally expected values for reasonable BBB permeability as compared to NAP (c Log P=1.18, c Log D=0.98 and pKa=7.19).

### Chemical Synthesis

All newly designed compounds were synthesized according to previously reported procedures (Li, et al. J. Med. Chem. 2009, 52, 1416-1427; Ma, et al. J. Med. Chem. 2019, 62, 11399-11415; Obeng, et al. ACS Chem. Neurosci. 2019, 10, 1075-1090; Yuan, et al. Bioorg. Med. Chem. 2015, 23, 1701-1715). Briefly, 6α- and 6β-naltrexamine (NTA) were synthesized by stereoselective reduction amination of naltrexone with benzylamine and dibenzylamine, respectively, followed by catalytic hydrogenation under acidic conditions. Various commercially available 5-membered heterocyclic carboxylic acids were coupled with 6α- and 6β-naltrexamine utilizing the EDCI/HOBt coupling reaction under mild basic conditions. 6-Position monosubstituted free bases were then obtained in reasonable yields by treating with K₂CO₃ in methanol (Scheme 1). These final compounds, obtained in yields ranging from 30-95%, were converted to their hydrochloric acid salt forms, fully characterized, and applied for in vitro and in vivo pharmacological characterization.

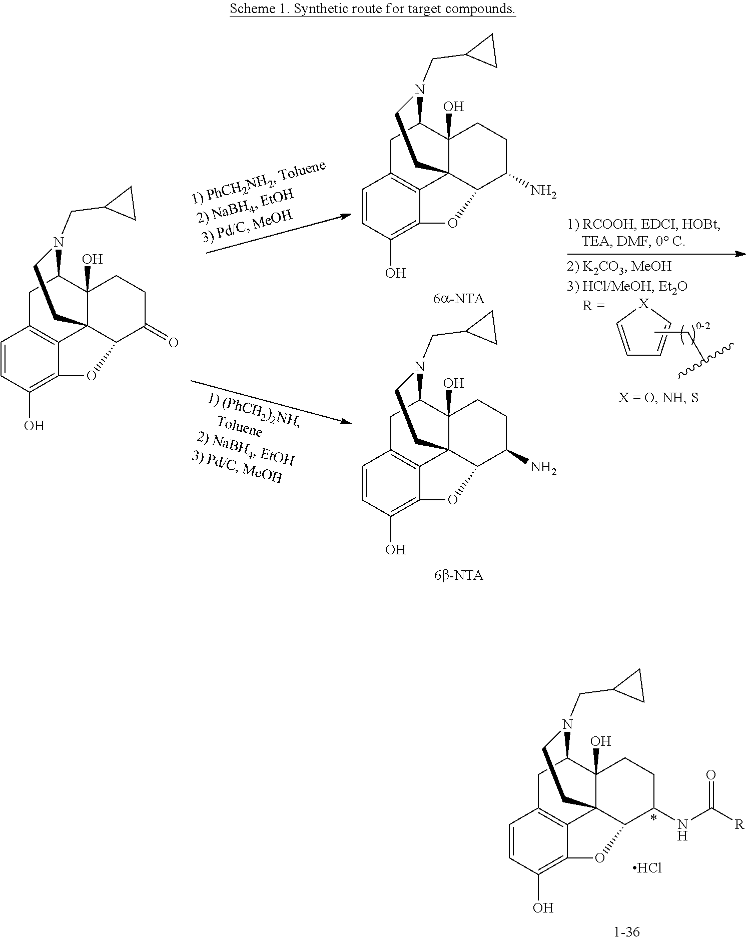

### In Vitro Pharmacological Studies

Binding affinity and selectivity of all synthesized ligands on the three opioid receptors were determined using the competitive radioligand binding assay. Following the previously reported protocol (Li, Ma and Yuan, supra), opioid receptor-expressing CHO cell membranes were used where the MOR was labelled with [³H]naloxone, the KOR and DOR with [³H]diprenorphine, respectively. The [³⁵S]GTPγS functional assay was then carried out to determine the agonist potency and efficacy of each ligand at MOR by measuring its efficacy relative to the full agonist DAMGO for MOR activation. The binding, selectivity, potency, and efficacy results for the 6α-analogs were summarized in Table 1 and the results for 6β-analogs were summarized in Table 2.

TABLE 1
Opioid receptor binding affinity and MOR [³⁵S]GTPγS functional assay results for 6α-analogs.

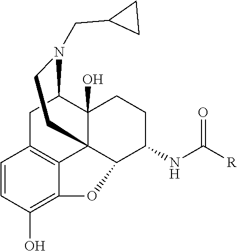

| | | Ki (nM) | | | Selectivity | | MOR [³⁵S] GTPγS Binding | |
|---|---|---|---|---|---|---|---|---|
| Compds | R | MOR | KOR | DOR | δ/μ | κ/μ | EC₅₀ (nM) | % Eₘₐₓ of DAMGO |
| NLX^{a} | — | 0.79 ± 0.02 | 1.1 ± 0.03 | 76 ± 2 | 69 | 1.0 | NA | 13 ± 1 |
| NAP^{b} | 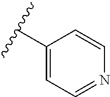 [SMILES: *c1ccncc1] | 0.37 ± 0.07 | 60.7 ± 5.6 | 277.5 ± 8.0 | 747 | 163 | 1.14 ± 0.38 | 22.72 ± 0.84 |
| 1 | 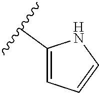 [SMILES: *c1ccc[nH]1] | 0.32 ± 0.03 | 1.58 ± 0.16 | 24.66 ± 2.22 | 76 | 5 | 3.39 ± 1.29 | 12.11 ± 2.64 |
| 3 | 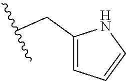 [SMILES: *Cc1ccc[nH]1] | 0.46 ± 0.04 | 2.88 ± 0.38 | 3.85 ± 1.18 | 8 | 6 | 4.18 ± 1.21 | 25.01 ± 2.61 |
| 5 | 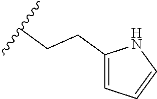 [SMILES: *CCc1ccc[nH]1] | 0.58 ± 0.03 | 0.54 ± 0.09 | 7.58 ± 0.37 | 13 | 1 | 3.79 ± 1.01 | 23.97 ± 3.59 |
| 7 | 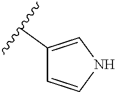 [SMILES: *c1cc[nH]c1] | 1.31 ± 0.09 | 2.78 ± 0.18 | 40.38 ± 5.80 | 31 | 2 | 2.21 ± 0.68 | 16.65 ± 1.25 |
| 9 | 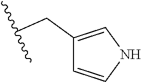 [SMILES: *Cc1cc[nH]c1] | 3.62 ± 0.43 | 19.0 ± 1.28 | 14.45 ± 1.81 | 4 | 5 | 51.36 ± 23.29 | 31.56 ± 2.87 |
| 11 | 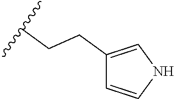 [SMILES: *CCc1cc[nH]c1] | 0.70 ± 0.12 | 0.59 ± 0.07 | 8.62 ± 1.37 | 12 | 1 | 3.86 ± 1.39 | 25.24 ± 3.26 |
| 13 | 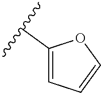 [SMILES: *c1ccco1] | 0.53 ± 0.33 | 1.97 ± 0.26 | 33.27 ± 3.51 | 63 | 4 | 9.49 ± 6.29 | 21.80 ± 5.19 |
| 15 | 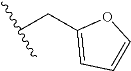 [SMILES: *Cc1ccco1] | 0.59 ± 0.03 | 6.67 ± 1.09 | 4.61 ± 0.42 | 8 | 12 | 2.74 ± 0.57 | 22.65 ± 2.77 |
| 17 | 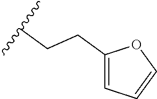 [SMILES: *CCc1ccco1] | 0.72 ± 0.10 | 1.52 ± 0.27 | 2.94 ± 0.26 | 4 | 2 | 2.77 ± 0.73 | 21.77 ± 2.76 |
| 19 | 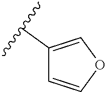 [SMILES: *c1ccoc1] | 0.50 ± 0.05 | 2.14 ± 0.36 | 28.63 ± 3.22 | 57 | 4 | 7.63 ± 4.22 | 18.14 ± 2.95 |
| 21 | 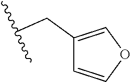 [SMILES: *Cc1ccoc1] | 0.42 ± 0.04 | 6.45 ± 0.59 | 3.74 ± 0.49 | 9 | 15 | 8.32 ± 3.56 | 14.95 ± 1.46 |
| 23 | 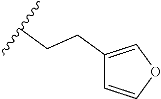 [SMILES: *CCc1ccoc1] | 0.44 ± 0.10 | 0.78 ± 0.04 | 3.54 ± 0.50 | 9 | 2 | 2.31 ± 0.87 | 27.62 ± 1.96 |
| 25 | 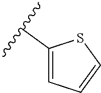 [SMILES: *c1cccs1] | 0.37 ± 0.01 | 1.59 ± 0.17 | 24.28 ± 5.08 | 65 | 4 | 0.44 ± 0.09 | 24.00 ± 2.89 |
| 27^{c} | 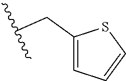 [SMILES: *Cc1cccs1] | 0.46 ± 0.05 | 6.04 ± 0.83 | 6.92 ± 1.63 | 15 | 13 | 5.95 ± 1.67 | 25.14 ± 1.07 |
| 29 | 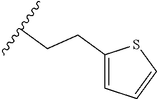 [SMILES: *CCc1cccs1] | 0.48 ± 0.03 | 1.76 ± 0.21 | 2.25 ± 0.43 | 7 | 4 | 3.06 ± 0.48 | 43.73 ± 1.71 |
| 31 | 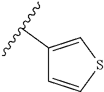 [SMILES: *c1ccsc1] | 0.38 ± 0.04 | 2.80 ± 0.12 | 10.71 ± 3.56 | 29 | 7 | 1.07 ± 0.22 | 30.66 ± 4.44 |
| 33 | 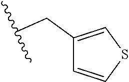 [SMILES: *Cc1ccsc1] | 0.23 ± 0.01 | 0.65 ± 0.08 | 39.41 ± 11.35 | 173 | 3 | 1.21 ± 0.40 | 28.59 ± 1.15 |
| 35 | 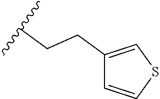 [SMILES: *CCc1ccsc1] | 0.23 ± 0.02 | 3.66 ± 0.44 | 18.55 ± 3.19 | 81 | 16 | 4.30 ± 2.29 | 33.40 ± 4.46 |

^{a}The in vitro data for NLX adopted from Peng, et al. J. Med. Chem. 2007, 50, 2254-2258.
^{b}The in vitro data for NAP adopted from Li et al. supra.
^{c}Compound first published in Ghirmai, et al. J. Med. Chem. 2008, 51, 1913-1924.

Overall, the 6α-analogs (except compounds 7 and 9) showed sub nanomolar affinity for the MOR, similar to that seen for NAP. Interestingly, the isosteric replacement of the address moiety of NAP, with pyrrole, furan and thiophene, in fact, resulted in improving KOR affinity leading to reduced selectivity between the MOR and KOR (Table 1). All analogs showed relatively higher affinity (single-to-double digit nanomolar Kᵢ) at the DOR compared to NAP. It was observed that the change between three isosteres, pyrrole, furan, and thiophene, resulted in no significant change in the binding affinity and selectivity for the MOR over KOR and DOR. For example, compounds 1 (Kᵢ κ/μ 4.85 and δ/μ 75.87), 13 (Kᵢ κ/μ 3.83 and δ/μ62.83) and 25 (Kᵢ κ/μ 4.25 and δ/μ 65.00) that differ only in the aromatic moiety in their address portion showed similar affinity and selectivity profiles. A similar trend was observed for all 6α-analogs. Compared to NAP, the compounds with n-propamido linker showed increased KOR affinity, thereby lowering the κ/μ selectivity, making these analogs less selective between the KOR and MOR than the ones with a carboxamido or acetamido spacer. In general, the compounds with an acetamido linker exhibited highest MOR-over-KOR selectivity.

Lastly, the attachment position of the linker, 2′ or 3′ of the aromatic ring, seemed to have little effect on either binding affinity or selectivity.

TABLE 2
Opioid receptor binding affinity and MOR [³⁵S]GTPγyS functional assay results for 6β-analogs.

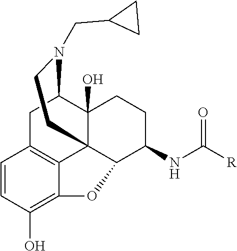

| | | Ki (nM) | | | Selectivity | | MOR [³⁵S]GTPγYS Binding | |
|---|---|---|---|---|---|---|---|---|
| Compds | R | MOR | KOR | DOR | δ/μ | κ/μ | EC₅₀ (nM) | % Eₘₐₓ of DAMGO |
| NLXª | — | 0.79 ± 0.02 | 1.1 ± 0.03 | 76 ± 2 | 69 | 1.0 | NA | 13 ± 1 |
| NAP^{b} | 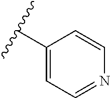 [SMILES: *c1ccncc1] | 0.37 ± 0.07 | 277.5 ± 8.0 | 60.7 ± 5.6 | 74 | 16 | 1.14 ± 0.38 | 22.72 ± 0.84 |
| 2 | 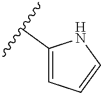 [SMILES: *c1ccc[nH]1] | 14.05 ± 0.95 | 40.70 ± 2.64 | 1639 ± 349 | 117 | 3 | 3.39 ± 1.29 | 12.43 ± 1.05 |
| 4 | 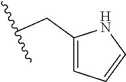 [SMILES: *Cc1ccc[nH]1] | 0.16 ± 0.02 | 1.35 ± 0.12 | 24.27 ± 4.51 | 152 | 8 | 0.80 ± 0.03 | 44.17 ± 1.52 |
| 6 | 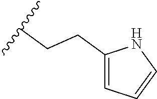 [SMILES: *CCc1ccc[nH]1] | 0.52 ± 0.08 | 1.25 ± 0.13 | 36.71 ± 4.24 | 70 | 2 | 1.22 ± 0.12 | 39.34 ± 3.16 |
| 8 | 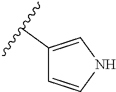 [SMILES: *c1cc[nH]c1] | 0.36 ± 0.04 | 4.51 ± 0.44 | 107.45 ± 16.44 | 298 | 12 | 2.03 ± 0.23 | 17.36 ± 2.04 |
| 10 | 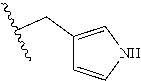 [SMILES: *Cc1cc[nH]c1] | 0.28 ± 0.05 | 4.19 ± 0.46 | 37.78 ± 5.61 | 135 | 15 | 1.18 ± 0.06 | 53.26 ± 0.95 |
| 12 | 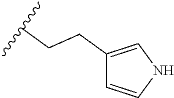 [SMILES: *CCc1cc[nH]c1] | 0.41 ± 0.08 | 2.72 ± 0.39 | 67.47 ± 11.18 | 164 | 7 | 1.60 ± 0.17 | 37.72 ± 0.87 |
| 14 | 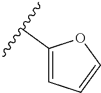 [SMILES: *c1ccco1] | 0.30 ± 0.05 | 1.29 ± 0.12 | 84.68 ± 4.75 | 282 | 4 | 0.98 ± 0.09 | 24.2 ± 0.9 |
| 16 | 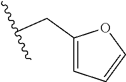 [SMILES: *Cc1ccco1] | 0.23 ± 0.03 | 3.38 ± 0.27 | 45.60 ± 5.12 | 198 | 15 | 0.97 ± 0.09 | 40.00 ± 0.9 |
| 18 | 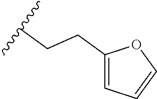 [SMILES: *CCc1ccco1] | 0.28 ± 0.02 | 0.61 ± 0.07 | 33.00 ± 3.58 | 118 | 2 | 1.33 ± 0.15 | 44.53 ± 2.72 |
| 20 | 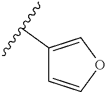 [SMILES: *c1ccoc1] | 8.47 ± 0.4 | 29.36 ± 2.80 | 1980 ± 271 | 234 | 3 | 34.99 ± 5.02 | 24.99 ± 1.74 |
| 22 | 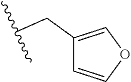 [SMILES: *Cc1ccoc1] | 0.22 ± 0.03 | 3.68 ± 0.13 | 73.02 ± 7.08 | 332 | 17 | 0.91 ± 0.22 | 30.83 ± 2.07 |
| 24 | 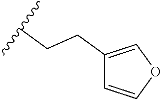 [SMILES: *CCc1ccoc1] | 0.21 ± 0.01 | 0.47 ± 0.03 | 32.70 ± 3.47 | 156 | 2 | 0.63 ± 0.12 | 42.61 ± 2.61 |
| 26 | 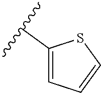 [SMILES: *c1cccs1] | 0.24 ± 0.01 | 0.79 ± 0.07 | 48.67 ± 1.62 | 206 | 3 | 0.54 ± 0.14 | 24.42 ± 2.04 |
| 28 | 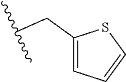 [SMILES: *Cc1cccs1] | 0.24 ± 0.02 | 1.61 ± 0.2 | 64.93 ± 2.38 | 270 | 7 | 1.16 ± 0.35 | 25.24 ± 2.33 |
| 30 | 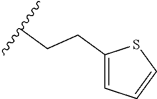 [SMILES: *CCc1cccs1] | 0.26 ± 0.01 | 0.23 ± 0.02 | 4.87 ± 1.19 | 19 | 1 | 1.52 ± 0.37 | 37.79 ± 4.30 |
| 32 | 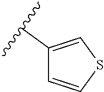 [SMILES: *c1ccsc1] | 0.25 ± 0.03 | 0.37 ± 0.06 | 152.9 ± 34.3 | 612 | 1 | 0.95 ± 0.3 | 26.49 ± 1.9 |
| 34 | 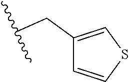 [SMILES: *Cc1ccsc1] | 0.20 ± 0.02 | 1.31 ± 0.18 | 36.11 ± 10.17 | 176 | 6 | 3.24 ± 0.11 | 26.49 ± 3.17 |
| 36 | 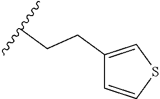 [SMILES: *CCc1ccsc1] | 0.21 ± 0.02 | 0.22 ± 0.04 | 51.01 ± 19.27 | 245 | 1 | 0.81 ± 0.08 | 64.66 ± 4.31 |

^{a}The in vitro data for NLX adopted from Peng et al. supra.
^{b}The in vitro data for NAP adopted from Li et al. supra.
^{c}Compound first published in Ghirmai et al. supra.

Among the 6β-analogs, it was observed that all compounds (except compounds 2 and 20) showed sub nanomolar affinity for the MOR, nanomolar affinity for the KOR and much lower affinity for the DOR (Table 2). Similar to their 6α-counterparts, these compounds also showed improved affinity for the KOR compared to NAP. The 6β-analogs, overall, showed much higher selectivity for the MOR over the DOR compared to their 6α-counterparts. Compound 32, with 3-thiophene in the address region showed the highest selectivity for the MOR over the DOR (Kᵢ δ/μ 612.0) while compound 22 with 2-furanylmethyl in the address region showed the highest selectivity for the MOR over the KOR (Kᵢ κ/˜ 16.7). The 6β-analogs with an acetamido linker presented higher MOR over KOR selectivity than the ones with a carboxamido or n-propamido linker.

As seen in Table 1, all 6α-analogs exhibited partial agonism with similar potency and efficacy. In detail, compounds 9, 29, 31 and 35 show higher efficacy than NAP, ranging between 30-50%, whereas compounds 1, 7, 19 and 21 showed lower efficacy than NAP (<20%). The remaining 6α-analogs (3, 5, 11, 13, 15, 17, 19, 23, 25, 27, 33) showed efficacies similar to NAP (20-30%) for G-protein activation in MOR-expressing CHO cells. There appeared to be no significant effect of chain length or substitution position on the heterocyclic rings on their efficacies.

Among the 6β-analogs, all compounds except compounds 2 and 8 exhibited partial agonism with efficacies ranging between 25-65%. Compounds 2 and 8 showed low efficacy (2, Eₘₐₓ=12.43±1.05; 8, Eₘₐₓ=17.36±2.04) and were identified as antagonists. Interestingly, both compounds 2 and 8 are pyrrole derivatives with a methyl linker and differ only in the substitution position of the linker on the pyrrole ring.

Overall, except compounds 2 and 20, substituting the pyridine ring in the address region of NAP with its isosteres pyrrole, furan and thiophene rings retained the high binding affinity as well as similar efficacy at the MOR.

### In Vivo Warm-Water Tail Immersion Assay

The warm-water tail immersion assay was conducted on all compounds to assess their antinociception potency and antagonism against morphine's antinociception. In this assay, the duration for which mice kept their tails in the warm water was recorded. The longer the duration, giving higher percent maximum possible effects (% MPE), the higher the antinociceptive effects the studied compound possesses.

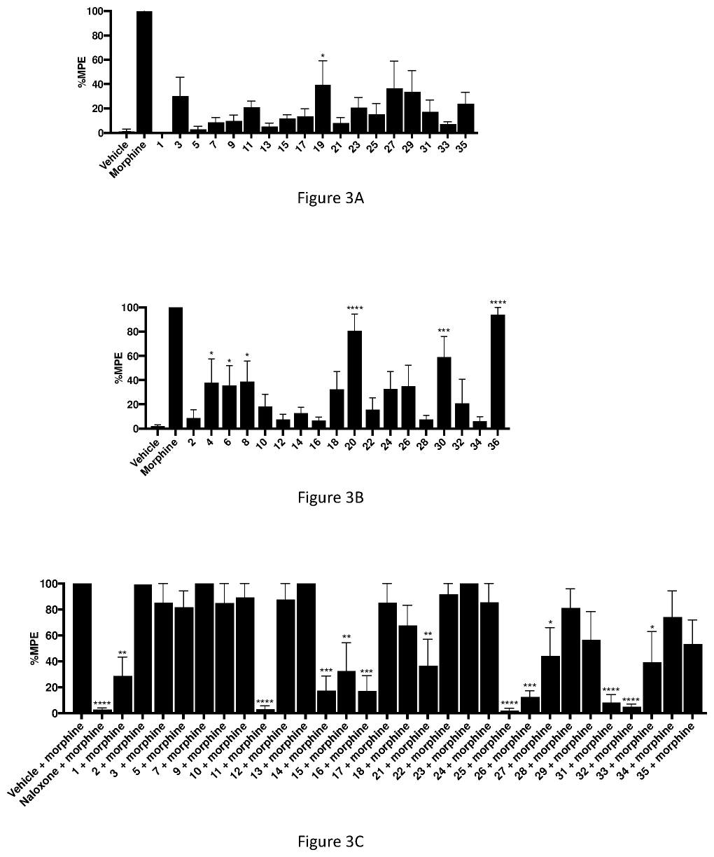

All thirty-six compounds' antinociception were examined in tail immersion assay first to preclude any opioid receptor agonists. In this study, the test was conducted 20 min after each compound (10 mg/kg) was injected subcutaneously. As shown in FIGS. 3A and 3B, among the 36 compounds, most compounds showed no significant antinociceptive effects compared to vehicle, which corresponded to their low efficacy at the MOR (Table 1 and 2). Compounds 4, 6, 8, 19, 20, 30, 36 exhibited antinociception which is reflected in their increased % MPE (FIGS. 3A and 3B). Among them, compounds 4, 6, 30 and 36 showed moderate efficacy at the MOR (Table 2), suggesting that their antinociceptive effects were most likely due to the activation at MOR. On the other hand, compounds 8 and 19, acted as low-efficacy MOR agonists in the [³⁵S]GTPγS binding assay while showing high KOR affinity (Table 1 and 2) suggesting that their antinociception may come from interacting with the KOR. Compound 20, however, demonstrated not only low efficacy and low potency at MOR, but also low to moderate binding affinity towards the DOR and KOR (Table 2). Hence, there might be mechanisms other than opioid receptor agonism responsible for the antinociception for 20. All other analogs showed no significant antinociception when compared to morphine and could potentially act as opioid antagonists.

The analogs defined as potential antagonists at MOR were then studied for their ability to antagonize morphine's antinociceptive effect. As seen from FIG. 3C, compounds 1, 11, 14, 15, 16, 25, 26, 31, and 32 significantly antagonized morphine's antinociceptive effect thereby showing pronounced antagonism of CNS antinociception. Interestingly, of the nine compounds identified as antagonists, six of them (compounds 1, 14, 25, 26, 31 and 32) possessed no linker methylene group between the amide bond to the address region. All remaining compounds (2, 3, 5, 7, 9, 10, 12, 13, 17, 18, 21, 22-24, 27-29, 33-35) did not produce any significant antinociception nor were they able to antagonize morphine's antinociceptive effect. It was observed that these compounds, except compounds 29 and 35, showed predicted c log P<2.5. Taking their high in vitro binding affinity to the MOR into account, these compounds most likely lack CNS permeability which resulted in their lack of in vivo activity.

Following this single dose assessment, in vivo dose-response studies with eight identified antagonists were conducted. Compound 11 exhibited poor solubility in pyrogen-free isotonic saline as well as sterile-filtered distilled/deionized water at higher doses while addition of 10% DMSO or 2% Tween80® did not improve its solubility significantly. Hence 11 was excluded from the following studies. The anti-antinociception potencies of remaining eight compounds were determined where their AD₅₀ values ranged from 0.42 to 23.54 mg/kg. Six out of eight identified antagonists, except 15 and 16, possessed AD₅₀ values comparable to NAP (Table 3). In fact, compounds 25, 26 and 31 were significantly more potent than NAP with 25 (AD₅₀=0.42 mg/kg) showing 10-fold higher potency (Table 3). Also, compound 25 showed much higher potency than other NAP derivatives, i.e. NFP (AD₅₀=2.82 mg/kg) and NYP (AD₅₀=1.75 mg/kg), which were identified in previous studies.³⁶ With the exception of compound 32, the predicted CNS relevant physicochemical properties also correlated well with the potency wherein compounds 11, 14, 15 and 16 showed c log P<2.5 and c log D<1.5 whereas compounds 25, 26 and 31 showed identical physicochemical properties (c Log P=2.77, c Log D=2.11) that predict higher CNS-permeability.

TABLE 3
AD₅₀ values of compounds to antagonize
morphine mediated antinociception.

| Compound | AD₅₀ mg/kg (95% CL) | |
|---|---|---|
| NLX^{a} | 0.05 | (0.03-0.09) |
| NAP^{b} | 4.51 | (2.45-8.26) |
| 1 | 4.71 | (1.61-13.81) |
| 14 | 6.04 | (3.47-10.50) |
| 15 | 12.31 | (6.92-21.90) |
| 16 | 23.54 | (11.34-48.83) |
| 25 | 0.42 | (0.21-0.82) |
| 26 | 1.62 | (1.12-2.36) |
| 31 | 1.51 | (1.08-2.09) |
| 32 | 6.81 | (2.46-18.91) |

^{a}The in vitro data for NLX adopted from Peng et al. supra.
^{b}The in vitro data for NAP adopted from Li et al. supra.

### KOR and DOR [³⁵S]GTPγS Binding Assays

As predicted and observed from the binding assay results (Table 2), many compounds resulted in decreased selectivity over the KOR and DOR compared to NAP, which we wondered could potentially lead to some undesired off-target effects. Hence before investigating their pharmacology further, the functionalities of the eight selected antagonists (1, 14-16, 25, 26, 31, and 32) on the KOR and DOR were investigated. In the KOR [³⁵S]GTPγS binding assays, all compounds showed moderate-to-high efficacy with single- or double-digit nanomolar potencies (Table 4). As KOR agonists may help treat morphine or oxycodone addiction and opioid-induced pruritus, the partial agonism exhibited by these compounds on the KOR may in fact be beneficial in OUD treatments. On the other hand, the high potency and efficacy of compound 32 towards the KOR, which was not observed in the in vivo antinociception study, could be concerning clinically as a full KOR agonist could also elicit dysphoria and sedation. Except for compound 15, all other compounds remained highly selective over the DOR with none displaying high potency or high efficacy in the DOR functional study (Table 4). Thus, although compound 15 showed relatively low δ/μ selectivity and a nanomolar level EC₅₀, we speculated that its partial agonism displayed at the DOR would not result in severe side effects such as convulsion. Therefore, the comparatively low κ/μ and δ/μ selectivity seemed acceptable for further pursuing these MOR ligands as potential therapeutic agents for OUD except of compound 32.

TABLE 4
Potencies and efficacies at KOR and DOR of
compounds 1, 14-16, 25, 26, 31, and 32.

| | | | KOR [³⁵S]GTPγS Binding | | DOR [³⁵S]GTPγS Binding | |
|---|---|---|---|---|---|---|
| | Selectivity | | | % Eₘₐₓ of | | % Eₘₐₓ of |
| Compd. | κ/μ | δ/μ | EC₅₀ (nM) | U50,488H | EC₅₀ (nM) | SNC80 |
| NAP^{a} | 163 | 747 | 28.8 ± 14.4 | 45.5 ± 4.4 | 15.2 ± 15.2 | 10.2 ± 3.1 |
| 1 | 5 | 76 | 14.8 ± 0.99 | 43.4 ± 1.36 | 26.9 ± 3.23 | 50.9 ± 5.25 |
| 14 | 4 | 282 | 6.3 ± 0.85 | 67.2 ± 2.53 | 35.5 ± 8.71 | 23.1 ± 0.97 |
| 15 | 12 | 8 | 85.4 ± 6.29 | 63.3 ± 2.50 | 6.2 ± 0.83 | 66.8 ± 4.24 |
| 16 | 15 | 198 | 13.3 ± 0.59 | 49.3 ± 1.07 | 42.3 ± 6.03 | 31.2 ± 1.49 |
| 25 | 4 | 65 | 1.7 ± 0.24 | 50.6 ± 0.95 | 59.4 ± 6.71 | 48.6 ± 3.41 |
| 26 | 3 | 206 | 7.0 ± 0.31 | 67.4 ± 3.00 | 70.4 ± 38.51 | 43.6 ± 7.31 |
| 31 | 7 | 29 | 16.3 ± 1.73 | 47.8 ± 0.85 | 38.8 ± 5.83 | 58.5 ± 3.13 |
| 32 | 1 | 612 | 1.0 ± 0.44 | 90.8 ± 2.34 | 31.4 ± 4.89 | 47.8 ± 0.95 |

^{a}The in vitro data for NAP adopted from Li et al. supra.

### In Vivo Opioid Withdrawal Studies

Opioid antagonists such as naloxone (NLX) and naltrexone are associated with significant withdrawal symptoms when administered to opioid dependent patients. Such drawbacks have limited the clinical application of naloxone and naltrexone. Since compounds 25, 26, and 31 appeared to the most potent antagonists in vivo among others, they were selected to be studied for their potency to produce withdrawal symptoms. Somatic symptoms of opioid withdrawal including wet-dog shakes, jumps and paw tremors were monitored and recorded over a period of 20 min, starting 3 min after each injection with the tested compounds given to morphine-pelleted mice. As shown in FIG. 3, NLX precipitated withdrawal symptoms at 1 mg/kg similar to previously reported.

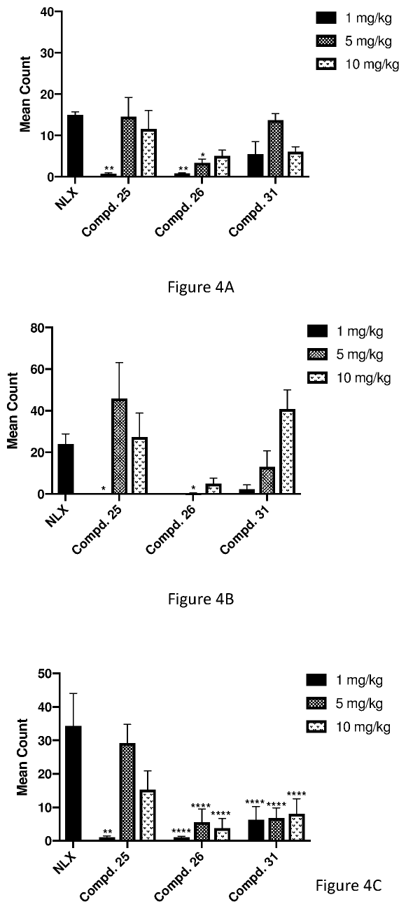

At 1 mg/kg dose, compounds 25, 26 and 31 produced significantly fewer wet-dog shakes, escape jumps, and paw tremors than 1 mg/kg NLX (FIG. 4A-C) in morphine pelleted mice. Both 6α-analogs, 25 and 31 started showing wet dog shakes and jumps at a 5 mg/kg dose. While paw tremoring was noteworthy for compound 25 at 5 mg/kg, it remained mild through all doses for compound 31. Interestingly, the 6β-analog 26 did not precipitate significant withdrawal symptoms at doses as high as 10 times that of NLX. Interestingly, compound 25 showed fewer withdrawal symptoms at 10 mg/kg compared to 5 mg/kg as well as observed for wet dog shakes seen for compound 31. We have observed some similar cases previously in our studies with other analogs which could be due to the nature of behavioral in vivo study and the individual conditions of mice. Meanwhile, the symptoms shown at both doses 5 mg/kg and 10 mg/kg were not significantly different statistically. Additionally, wet dog shakes and jumps were seen at 20 and 33.8 mg/kg equivalent to those seen for 1 mg/kg of naloxone (data not shown). Although the compounds exhibit lower potency than NLX, the partial agonism shown by the new analogs compared to NLX's neutral antagonism could be a reason of their reduced withdrawal symptoms. Overall, the results suggest that these compounds, especially compound 26, precipitate much less withdrawal effects than NLX thus making them promising candidates to develop for the treatment for opioid use disorders.

### BBB-Penetration Studies

As our goal is to develop centrally acting MOR antagonists, we designed NAP analogs by applying the isosteric replacement strategy and physicochemical parameters predictions. Additionally, CNS penetrance of selected compounds was further estimated using other models including Swiss ADME, Pfizer's central nervous system multiparameter optimization (CNS-MPO) index and ligand-lipophilic efficiency indices (LLE). Swiss ADME predicted compounds 25, 26 and 31 to be CNS-non permeant. CNS-MPO estimates a score higher than 4 as criterion for CNS hit selection. Determination these scores for these compounds revealed naltrexone (MPO score 5.5) and NAP (MPO score 4.4) to be CNS-permeable while compounds 25, 26, and 31 (MPO score 3.8) to be CNS-non permeable (Table 5 and Table 6). Similarly, determination of the LLE indicated that NAP (PNS-acting) has higher index of 8.3 while NLX (LLE 7.0) and compounds 25 (LLE 6.7), 26 (LLE 6.85) and 31 (6.65) showed slightly lower indices (Table 5 and Table 6). While compounds 25, 26 and 31 seemed significantly more potent than NAP in vivo, MPO and LLE scores appeared to have their limitations to predict the CNS permeability of NAP analogs.

TABLE 5
In-silico physicochemical properties prediction of final compounds

| | | | pKa | cLogD | | | |
|---|---|---|---|---|---|---|---|
| Compound | Mol. wt. | cLogP | (Base) | (pH 7.4) | HBD | HBA | TPSA |
| NAP | 447.54 | 1.18 | 7.17 | 0.98 | 3 | 7 | 94.39 |
| 1 | 435.52 | 1.71 | 7.39 | 1.35 | 4 | 7 | 97.82 |
| 2 | 435.52 | 1.71 | 7.39 | 1.35 | 4 | 7 | 97.82 |
| 3 | 449.54 | 1.88 | 7.35 | 1.02 | 4 | 7 | 97.82 |
| 4 | 449.54 | 1.88 | 7.35 | 1.02 | 4 | 7 | 97.82 |
| 5 | 463.57 | 2.31 | 7.37 | 1.50 | 4 | 7 | 97.82 |
| 6 | 463.57 | 2.31 | 7.37 | 1.50 | 4 | 7 | 97.82 |
| 7 | 435.52 | 1.71 | 7.36 | 0.94 | 4 | 7 | 97.82 |
| 8 | 435.52 | 1.71 | 7.36 | 0.94 | 4 | 7 | 97.82 |
| 9 | 449.54 | 1.88 | 7.36 | 1.01 | 4 | 7 | 97.82 |
| 10 | 449.54 | 1.88 | 7.36 | 1.01 | 4 | 7 | 97.82 |
| 11 | 463.57 | 2.31 | 7.37 | 1.50 | 4 | 7 | 97.82 |
| 12 | 463.57 | 2.31 | 7.37 | 1.50 | 4 | 7 | 97.82 |
| 13 | 436.50 | 1.99 | 7.35 | 1.44 | 3 | 7 | 95.17 |
| 14 | 436.50 | 1.99 | 7.35 | 1.44 | 3 | 7 | 95.17 |
| 15 | 450.53 | 2.22 | 7.35 | 1.50 | 3 | 7 | 95.17 |
| 16 | 450.53 | 2.22 | 7.35 | 1.50 | 3 | 7 | 95.17 |
| 17 | 464.55 | 2.47 | 7.37 | 1.87 | 3 | 7 | 95.17 |
| 18 | 464.55 | 2.47 | 7.37 | 1.87 | 3 | 7 | 95.17 |
| 19 | 436.50 | 1.99 | 7.30 | 1.51 | 3 | 7 | 95.17 |
| 20 | 436.50 | 1.99 | 7.30 | 1.51 | 3 | 7 | 95.17 |
| 21 | 450.53 | 2.22 | 7.36 | 1.50 | 3 | 7 | 95.17 |
| 22 | 450.53 | 2.22 | 7.36 | 1.50 | 3 | 7 | 95.17 |
| 23 | 464.55 | 2.47 | 7.37 | 1.87 | 3 | 7 | 95.17 |
| 24 | 464.55 | 2.47 | 7.37 | 1.87 | 3 | 7 | 95.17 |
| 25 | 452.57 | 2.77 | 7.34 | 2.11 | 3 | 6 | 110.27 |
| 26 | 452.57 | 2.77 | 7.34 | 2.11 | 3 | 6 | 110.27 |
| 27 | 466.59 | 2.65 | 7.34 | 1.92 | 3 | 6 | 110.27 |
| 28 | 466.59 | 2.65 | 7.34 | 1.92 | 3 | 6 | 110.27 |
| 29 | 480.62 | 3.04 | 7.37 | 2.40 | 3 | 6 | 110.27 |
| 30 | 480.62 | 3.04 | 7.37 | 2.40 | 3 | 6 | 110.27 |
| 31 | 452.57 | 2.77 | 7.34 | 2.11 | 3 | 6 | 110.27 |
| 32 | 452.57 | 2.77 | 7.34 | 2.11 | 3 | 6 | 110.27 |
| 33 | 466.59 | 2.65 | 7.36 | 1.92 | 3 | 6 | 110.27 |
| 34 | 466.59 | 2.65 | 7.36 | 1.92 | 3 | 6 | 110.27 |
| 35 | 480.62 | 3.04 | 7.37 | 2.39 | 3 | 6 | 110.27 |
| 36 | 480.62 | 3.04 | 7.37 | 2.39 | 3 | 6 | 110.27 |

Mol. wt., molecular weight; HBD, hydrogen bond donor; HBA, hydrogen bond acceptor; TPSA, total polar surface area.
Data generated using ACD/Percepta v2020.2.0.

TABLE 6
CNS-MPO and LLE calculations of most potent compounds

| Compound | CNS-MPO^{a} | LLE^{b} |
|---|---|---|
| NLX | 5.5 | 7.01 |
| NAP | 4.4 | 8.25 |
| 1 | 4.2 | 7.78 |
| 14 | 4.4 | 7.53 |
| 15 | 4.3 | 7.01 |
| 16 | 4.3 | 7.42 |
| 25 | 3.8 | 6.66 |
| 26 | 3.8 | 6.85 |
| 31 | 3.8 | 6.65 |
| 32 | 3.82 | 6.83 |

^{a}Values calculated using the CNS-MPO calculator
^{b}LLE = pK_{i (MOR)} − cLogP

Several factors govern in vivo efficacy including intrinsic clearance and efflux transport in addition to CNS-permeability. The low in vivo potency of NAP mainly is due to its poor CNS permeability. Although NAP was determined to be a P-gp substrate, efflux by P-gp was not the most critical issue of impeded CNS permeability for NAP's already very low diffusional permeability (P_{app, A-B}=0.6±0.17 and P_{app, B-A}=7.8±1.0 in units of 10⁻⁶ cm/s). The permeability of NAP was not lower than naltrexone but was similar to mannitol, a paracellular permeability marker. Therefore, we postulated that the major concern was physicochemical properties of NAP (c Log P=1.18, c Log D=0.98, pKa=7.17, TPSA=94.39) which hampered its passive permeability.

Subsequently, passive permeability of the most potent compound (25) was assessed. Compound 25 showed a P_{app, A-B}=11.9±0.91 10⁻⁶ cm/s and a P_{app, B-A}=26.5±0.86 10⁻⁶ cm/s thus suggesting it was highly permeable as compared to NAP. Further, in vivo time dependent BBB-penetration studies were carried out. Compound 25 was administered s.c. at the tested dose of 10 mg/kg following which mice were sacrificed at 5, 10 and 30 min and their plasma and blood samples were collected. After the blood samples were centrifuged to obtain plasma, the plasma and brain homogenate samples were then analyzed to determine the amount of compound 25 using liquid chromatography-tandem mass spectrometry (LC-MS/MS) and the brain-to-plasma ratios were calculated (Table 7). Compound 25 appeared in plasma with the highest concentration (4.13 g/mL) as early as 5 min after s.c. administration. Brain concentrations of compound 25 after 5, 10 and 30 min were 0.283, 0.366 and 0.459 μg/g respectively, indicating that compound 25 penetrated into the CNS after subcutaneous administration. Additionally, the brain-to-plasma concentration ratio of compound 25 increased over time indicating its progressive BBB-penetration.

TABLE 7
BBB Penetration of compound 25 (10 mg/kg, s.c) in
mice (n = 3, mean ± SD) at various time points.

| Time (min) | 5 | 10 | 30 |
|---|---|---|---|
| Brain (μg/g) | 0.283 ± 0.09 | 0.366 ± 0.08 | 0.459 ± 0.08 |
| Plasma (μg/mL) | 4.130 ± 1.31 | 3.150 ± 1.87 | 1.547 ± 0.96 |
| Brain-to-Plasma ratio | 0.068 | 0.116 | 0.297 |

### Conclusions

In summary, an isosteric ring replacement strategy was utilized to design a novel series of NAP derivatives as potential CNS-permeable and MOR-selective antagonists. This isosteric replacement aimed to improve CNS-permeability by substituting the pyridine ring in NAP with pyrrole, furan and thiophene systems. In general, all compounds retained high MOR binding affinity. It was observed that the heteroaromatic ring and position of substitution had no significant influence on the binding affinity and selectivity. However, the linker length and the configuration of C(6) seemed to affect their MOR selectivity over KOR and DOR. Moreover, from the in vivo studies, it was observed that at least 16 compounds (seven agonists and nine antagonists) showed improved CNS permeability, indicating the success of our isostere replacement as a lead modification strategy. Furthermore, out of the nine CNS-active MOR antagonists identified in the in vivo study, compounds 25, 26 and 31 demonstrated remarkable CNS antagonism against morphine and precipitated fewer withdrawal symptoms than NLX. Interestingly, all three compounds contain a thiophene moiety with no linker carbon (n=0) between the amide bond to the address moieties. These compounds also showed identical CNS relevant physicochemical properties (c Log P=2.77, c Log D=2.11, pKa=7.34) predicting them to be BBB permeable as compared to NAP (c Log P=1.18, c Log D=0.98 and pKa=7.19), which was further confirmed by in vivo BBB-penetration studies for the most potent compound 25. Thus, these novel thiophene isosteres of NAP showed promising potential for their utility in the treatment of opioid use disorders.

### Abbreviations Used

BBB, brain-blood barrier; cAMP, cyclic adenosine monophosphate; CHO, Chinese hamster ovary; CL, confidence level; CNS, central nervous system; DAMGO, [D-Ala2-MePhe4-Gly(ol)5]enkephalin; DOR, 6 opioid receptor; EDCI, 1-Ethyl-3-(3-dimethylaminopropyl)carbodiimide; GPCR, G protein-coupled receptor; HOBt, Hydroxybenzotriazole; KOR, κ opioid receptor; MOR, μ opioid receptor; NAP, 17-cyclopropylmethyl-3,14-dihydroxy-4,5a-epoxy-6β-[(4′-pyridyl)carboxamido]morphinan; NIDA, National Institute of Drug Abuse; NLX, naloxone; NOP, nociception/orphanin FQ receptor; NTA, naltrexamine; OUD, opioid use disorder; % MPE, percentage maximum possible effect.

### Experimental Section

### Chemistry.

All nonaqueous reactions were carried out under a pre-dried nitrogen gas atmosphere. Naloxone-d₅ was purchased from Cerilliant Corp. All other solvents and reagents were purchased from Sigma-Aldrich, Alfa Aesar, and Fisher Scientific, and were used as received without further purification. Melting points were measured on an MPA100 OptiMelt automated melting point apparatus without correction. IR spectra were recorded on a Thermo Scientific Nicolet iS10 FT-IR spectrometer. Analytical thin-layer chromatography (TLC) analyses were carried out on Analtech Uniplate F254 plates, and flash column chromatography (FCC) was performed over silica gel (230-400 mesh, Merck). ¹H (400 MHz) and ¹³C (100 MHz) nuclear magnetic resonance (NMR) spectra were recorded on a Bruker Ultrashield 400 Plus spectrometer, and chemical shifts were expressed in ppm. High resolution mass spectra were obtained on an Applied BioSystems 3200 Q trap with a turbo V source for TurbolonSpray. Analytical reversed-phase high-performance liquid chromatography (HPLC) was performed on a Varian ProStar 210 system using an Agilent Microsorb-MV 100-5 C18 column (250×4.6 mm). All analyses were conducted at ambient temperature with a flow rate of 0.8 mL/min. The mobile phase is acetonitrile/water (90:10) with 0.1% trifluoroacetic acid (TFA). The UV detector was set up at 210 nm. Compound purities were calculated as the percentage peak area of the analyzed compound, and retention times (Rt) were presented in minutes. The purity of all newly synthesized compounds was identified as >95%.

### General Procedure for the Amide Coupling/Hydrolysis Reaction.

A solution of carboxylic acid (2.5 equiv.) in dry DMF (1.5 mL) was added with hydrobenzotriazole (HOBt, 3 equiv.), N-(3-dimethylaminopropyl)-N′-ethylcarbodiimide hydrochloride (EDCI, 3 equiv.), 4 Å molecular sieves, and triethylamine (5 equiv.) on an ice-water bath. After 1 h, a solution of 6α-naltrexamine or 6β-naltrexamine (1 equiv.) in pre-dried DMF (1.5 mL) was added dropwise. The resulting mixture was stirred at room temperature. Once TLC indicated complete consumption of the starting material, the reaction mixture was filtered through celite. The filtrate was concentrated to dryness and dissolved in anhydrous methanol (3 mL), and then K₂CO₃ (2.5 equiv.) was added. The resulting mixture was stirred overnight at room temperature and filtered again over celite. After being concentrated, the residue was purified by flash column chromatography with CH₂Cl₂/MeOH (1% NH₃·H₂O) as the eluent to give the free base. After structural confirmation by ¹H NMR, the corresponding free base was then converted into a hydrochloride salt, which was fully characterized by ¹H NMR, ¹³C NMR, IR, HRMS, and HPLC.

### 17-Cyclopropylmethyl-3,14β-dihydroxy-4,5α-epoxy-6a-[(2′-pyrrolyl)carboxamido]morphinan hydrochloride (1)

Compound 1 was synthesized as shown in the general procedure with 37% yield. ¹H NMR (400 MHz, DMSO-d₆): δ 11.53 (s, 1H), 10.15 (s, 1H), 9.21 (s, 1H), 8.86 (s, 1H), 7.57 (d, J=8 Hz, 1H), 6.89 (m, 1H), 6.85 (m, 1H), 6.72 (d, J=8 Hz, 1H), 6.57 (d, J=8 Hz, 1H), 6.33 (s, 1H), 6.10 (m, 1H), 4.72 (d, J=3.8 Hz, 1H), 4.57 (m, 1H), 3.93 (d, J=6.8 Hz, 1H), 3.25 (m, 2H), 2.96 (m, 1H), 2.73 (m, 1H), 1.92 (m, 1H), 1.64 (m, 1H), 1.46 (m, 1H), 1.12 (m, 2H), 0.69 (m, 1H), 0.62 (m, 1H), 0.49 (m, 1H), 0.40 (m, 1H). ¹³C NMR (100 MHz, DMSO-d₆): δ 159.9, 146.0, 138.7, 128.7, 125.9, 122.0, 121.4, 119.0, 118.3, 110.9, 108.4, 87.5, 69.3, 61.0, 57.0, 45.3, 45.1, 45.1, 30.2, 29.2, 23.4, 8.4, 5.6, 5.1, 2.5. IR (diamond, cm⁻¹) vₘₐₓ: 3259, 1749, 1621, 1453, 1116, 1067, 1035, 748. HRMS: m/z calc. 436.2158 [M+H]⁺, obs. 436.2232 [M+H]⁺. The purity of the compound was checked by HPLC (Rt=7.56 min) and was found to be 97.72% pure.

### 17-Cyclopropylmethyl-3,14β-dihydroxy-4,5α-epoxy-6β-[(2′-pyrrolyl)carboxamido]morphinan hydrochloride (2)

Compound 2 was synthesized as shown in the general procedure with 38% yield. ¹H NMR (400 MHz, DMSO-d₆): δ 10.56 (s, 1H), 9.33 (s, 1H), 8.82 (s, 1H), 8.17 (d, J=8 Hz, 1H), 6.71 (d, J=8 Hz, 1H), 6.63 (d, J=8 Hz, 1H), 6.60 (m, 1H), 6.16 (s, 1H), 5.90 (m, 1H), 5.81 (m, 1H), 4.58 (d, J=8 Hz, 1H), 3.83 (d, J=4 Hz, 1H), 3.43 (s, 1H), 3.34 (m, 2H), 3.05 (m, 2H), 2.84 (m, 1H), 2.43 (m, 2H), 1.73 (m, 2H), 1.51 (m, 2H), 1.32 (m, 1H), 1.05 (m, 1H), 0.67 (m, 1H), 0.58 (m, 1H), 0.50 (m, 1H), 0.40 (m, 1H). ¹³C NMR (100 MHz, DMSO-d₆): δ 160.1, 157.5, 146.6, 133.2, 130.4, 128.2, 126.0, 120.1, 116.9, 109.9, 108.5, 91.2, 69.6, 61.5, 56.7, 50.6, 46.1, 45.5, 29.2, 27.4, 23.7, 23.3, 5.6, 5.0, 2.6. IR (diamond, cm⁻¹) vₘₐₓ: 3160, 1639, 1298, 1124, 748. HRMS: m/z calc. 436.2158 [M+H]⁺, obs. 436.2246 [M+H]⁺. The purity of the compound was checked by HPLC (Rt=6.45 min) and was found to be 96.98% pure.

### 17-Cyclopropylmethyl-3,14β-dihydroxy-4,5α-epoxy-6a-[²′-(2′-pyrrolyl)acetamido]morphinan hydrochloride (3)

Compound 3 was synthesized as shown in the general procedure with 53% yield. ¹H NMR (400 MHz, DMSO-d₆): δ 10.21 (s, 1H), 9.26 (s, 1H), 8.84 (s, 1H), 7.97 (d, J=8 Hz, 1H), 7.54 (m, 1H), 6.73 (d, J=8 Hz, 1H), 6.56 (d, J=8 Hz, 1H), 6.38 (m, 1H), 6.29 (s, 1H), 6.22 (m, 1H), 4.60 (d, J=4 Hz, 1H), 4.40 (m, 1H), 3.91 (d, J=8 Hz, 1H), 3.59 (s, 2H), 3.27 (m, 2H), 2.95 (m, 1H), 2.70 (m, 1H), 2.43 (m, 1H), 1.87 (m, 1H), 1.61 (m, 1H), 1.40 (m, 2H), 1.06 (m, 1H), 1.05 (m, 1H), 0.68 (m, 1H), 0.60 (m, 1H), 0.48 (m, 1H), 0.39 (m, 1H). ¹³C NMR (100 MHz, DMSO-d₆): δ 167.3, 150.0, 145.9, 141.7, 138.8, 128.7, 122.0, 119.0, 118.2, 110.4, 107.1, 87.3, 69.3, 60.9, 56.9, 45.3, 45.1, 34.9, 30.1, 29.1, 23.4, 19.6, 8.4, 5.6, 5.1, 2.5. IR (diamond, cm⁻¹) vₘₐₓ: 3236, 1634, 1455, 1116, 1066, 1032, 720. HRMS: m/z calc. 450.2315 [M+H]⁺, obs. 450.2397 [M+H]⁺. The purity of the compound was checked by HPLC (Rt=7.51 min) and was found to be 99.84% pure.

### 17-Cyclopropylmethyl-3,14β-dihydroxy-4,5α-epoxy-6β-[2′-(2′-pyrrolyl)acetamido]morphinan hydrochloride (4)

Compound 4 was synthesized as shown in the general procedure with 72% yield. ¹H NMR (400 MHz, DMSO-d₆): δ 10.56 (s, 1H), 9.33 (s, 1H), 8.83 (s, 1H), 8.16 (d, J=4 Hz, 1H), 6.72 (d, J=8 Hz, 1H), 6.64 (d, J=8 Hz, 1H), 6.61 (m, 1H), 6.16 (s, 1H), 5.91 (m, 1H), 5.83 (m, 1H), 4.60 (d, J=8 Hz, 1H), 3.85 (d, J=4 Hz, 1H), 3.05 (m, 2H), 2.85 (m, 1H), 2.43 (m, 1H), 1.72 (m, 2H), 1.53 (m, 1H), 1.43 (m, 1H), 1.34 (m, 1H), 1.07 (m, 1H), 0.68 (m, 1H), 0.59 (m, 1H), 0.52 (m, 1H), 0.43 (m, 1H). ¹³C NMR (100 MHz, DMSO-d₆): δ 169.2, 142.0, 141.2, 129.5, 125.2, 120.5, 119.2, 117.8, 116.7, 107.1, 105.8, 89.8, 69.6, 61.6, 56.6, 50.8, 46.4, 45.5, 35.0, 29.2, 27.3, 23.5, 22.9, 5.6, 5.0, 2.5. IR (diamond, cm⁻¹) vₘₐₓ: 3076, 1655, 1502, 1316, 1127, 1033, 726. HRMS: m/z calc. 450.2315 [M+H]⁺, obs. 450.2372 [M+H]⁺. The purity of the compound was checked by HPLC (Rt=7.34 min) and was found to be 97.71% pure.

### 17-Cyclopropylmethyl-3,14β-dihydroxy-4,5α-epoxy-6α[3′-(2′pyrrolyl)propanamido]morphinan hydrochloride (5)

Compound 5 was synthesized as shown in the general procedure with 72% yield. ¹H NMR (400 MHz, DMSO-d₆): δ 10.50 (s, 1H), 9.21 (s, 1H), 8.84 (s, 1H), 7.68 (d, J=8 Hz, 1H), 6.72 (d, J=8 Hz, 1H), 6.56 (m, 2H), 6.26 (s, 1H), 5.81 (m, 1H), 5.73 (m, 1H), 4.59 (m, 1H), 4.41 (m, 1H), 3.90 (m, 1H), 3.35 (m, 2H), 3.25 (m, 1H), 3.00 (m, 3H), 2.73 (m, 3H), 2.44 (m, 2H), 1.85 (m, 1H), 1.60 (m, 1H), 1.39 (m, 2H), 1.07 (m, 1H), 0.92 (m, 1H), 0.69 (m, 1H), 0.60 (m, 1H), 0.48 (m, 1H), 0.39 (m, 1H). ¹³C NMR (100 MHz, DMSO-d₆): δ 171.0, 145.9, 138.7, 130.8, 128.7, 122.0, 119.0, 118.1, 115.9, 107.0, 104.1, 87.5, 69.3, 60.9, 56.9, 45.1, 45.1, 44.8, 35.3, 30.1, 29.1, 23.4, 23.2, 19.6, 5.6, 5.1, 2.5. IR (diamond, cm⁻¹) vₘₐₓ: 3223, 1635, 1455, 1116, 1033, 729. HRMS: m/z calc. 464.2471 [M+H]⁺, obs. 464.2532 [M+H]⁺. The purity of the compound was checked by HPLC (Rt=7.58 min) and was found to be 99.65% pure.

### 17-Cyclopropylmethyl-3,14β-dihydroxy-4,5α-epoxy-6β-[3′-(2′-pyrrolyl)propanamido]morphinan hydrochloride (6)

Compound 6 was synthesized as shown in the general procedure with 71% yield. ¹H NMR (400 MHz, DMSO-d₆): δ 10.50 (s, 1H), 9.20 (s, 1H), 8.84 (s, 1H), 7.68 (d, J=8 Hz, 1H), 6.72 (d, J=8 Hz, 1H), 6.56 (m, 2H), 6.25 (s, 1H), 5.86 (m, 1H), 5.73 (m, 1H), 4.59 (d, J=4 Hz, 1H), 4.42 (m, 1H), 3.91 (m, 1H), 3.27 (m, 2H), 3.04 (m, 2H), 2.94 (m, 1H), 2.73 (m, 2H), 2.44 (m, 2H), 1.85 (m, 1H), 1.61 (m, 1H), 1.38 (m, 1H), 1.07 (m, 1H), 0.93 (m, 1H), 0.69 (m, 1H), 0.60 (m, 1H), 0.48 (m, 1H), 0.39 (m, 1H). ¹³C NMR (100 MHz, DMSO-d₆): δ 171.1, 142.1, 141.2, 130.7, 129.6, 120.5, 119.1, 117.8, 115.9, 107.0, 104.2, 89.9, 69.7, 61.6, 56.6, 50.5, 46.4, 45.5, 35.8, 29.3, 27.2, 23.6, 23.1, 8.4, 5.6, 5.0, 2.6. IR (diamond, cm⁻¹) vₘₐₓ: 3162, 1644, 1407, 1125, 1032, 746. HRMS: m/z calc. 464.2471 [M+H]⁺, obs. 464.2554 [M+H]⁺. The purity of the compound was checked by HPLC (RT=6.45 min) and was found to be 98.97% pure.

### 17-Cyclopropylmethyl-3,14β-dihydroxy-4,5α-epoxy-6α-[(3′-pyrrolyl)carboxamido]morphinan hydrochloride (7)

Compound 7 was synthesized as shown in the general procedure with 95% yield. ¹H NMR (400 MHz, DMSO-d₆): δ 11.52 (s, 1H), 9.21 (s, 1H), 8.84 (s, 1H), 7.56 (m, 1H), 6.89 (s, 1H), 6.84 (s, 1H), 6.71 (d, J=8 Hz, 1H), 6.58 (d, J=8 Hz, 1H), 6.31 (s, 1H), 6.11 (m, 1H), 4.73 (m, 1H), 4.55 (m, 1H), 3.91 (s, 1H), 3.29 (m, 2H), 2.95 (m, 1H), 2.72 (m, 1H), 1.91 (m, 1H), 1.64 (m, 1H), 1.45 (m, 2H), 1.25 (m, 1H), 1.10 (m, 2H), 0.70 (m, 1H), 0.61 (m, 1H), 0.49 (m, 1H), 0.40 (m, 1H). ¹³C NMR (100 MHz, DMSO-d₆): δ 161.1, 146.0, 145.3, 143.8, 138.7, 128.6, 122.5, 122.0, 119.0, 118.2, 109.2, 87.1, 69.3, 56.9, 45.4, 45.1, 30.3, 30.2, 29.1, 23.4, 19.2, 8.4, 5.6, 5.1, 2.5. IR (diamond, cm⁻¹) vₘₐₓ: 3240, 1654, 1540, 1115, 1033, 917, 746. HRMS: m/z calc. 436.2158 [M+H]⁺, obs. 436.2231 [M+H]⁺. The purity of the compound was checked by HPLC (Rt=6.44 min) and was found to be 99.20% pure.

### 17-Cyclopropylmethyl-3,14β-dihydroxy-4,5α-epoxy-6β-[(3′-pyrrolyl)carboxamido]morphinan hydrochloride (8)

Compound 8 was synthesized as shown in the general procedure with 74% yield. ¹H NMR (400 MHz, DMSO-d₆): δ 10.16 (s, 1H), 9.26 (s, 1H), 8.84 (s, 1H), 7.97 (d, J=8 Hz, 1H), 7.54 (m, 1H), 6.73 (d, J=8 Hz, 1H), 6.56 (d, J=8 Hz, 1H), 6.38 (m, 1H), 6.28 (s, 1H), 6.22 (m, 1H), 4.60 (d, J=4 Hz, 1H), 4.40 (m, 1H), 3.90 (d, J=8 Hz, 1H), 3.59 (m, 1H), 2.94 (m, 1H), 2.71 (m, 1H), 2.48-2.41 (m, 1H), 1.86 (m, 1H), 1.61 (m, 1H), 1.40 (m, 2H), 1.06 (m, 1H), 0.94 (m, 1H), 0.69 (m, 1H), 0.60 (m, 1H), 0.48 (m, 1H), 0.39 (m, 1H). ¹³C NMR (100 MHz, DMSO-d₆): δ 167.3, 150.0, 145.9, 141.7, 138.8, 128.7, 122.0, 119.0, 118.2, 110.4, 107.1, 87.3, 69.3, 60.9, 56.9, 45.3, 45.1, 34.9, 30.1, 29.1, 23.4, 19.6, 8.4, 5.6, 5.1, 2.5. IR (diamond, cm⁻¹) vₘₐₓ: 3046, 1634, 1500, 1125, 1032, 748. HRMS: m/z calc. 436.2158 [M+H]⁺, obs. 436.2231 [M+H]⁺. The purity of the compound was checked by HPLC (Rt=7.24 min) and was found to be 96.13% pure.

### 17-Cyclopropylmethyl-3,14β-dihydroxy-4,5α-epoxy-6α-[(3′-pyrrolyl)acetamido]morphinan hydrochloride (9)

Compound 9 was synthesized as shown in the general procedure with 76% yield. ¹H NMR (400 MHz DMSO-d₆): δ 10.51 (s, 1H), 9.23 (s, 1H), 8.81 (s, 1H), 7.72 (d, J=8 Hz, 1H), 6.72 (d, J=8 Hz, 1H), 6.60 (m, 1H), 6.56 (d, J=8 Hz, 1H), 6.22 (s, 1H), 5.90 (m, 1H), 5.82 (m, 1H), 4.59 (d, J=3.88 Hz, 1H), 4.39 (m, 1H), 3.87 (d, J=8 Hz, 1H), 3.44 (s, 2H), 3.40 (m, 1H), 3.29 (m, 1H), 3.05 (m, 2H), 2.93 (m, 1H), 2.70 (m, 1H), 2.43 (m, 1H), 1.84 (m, 1H), 1.62 (m, 1H), 1.40 (m, 2H), 1.04 (m, 1H), 0.93 (m, 1H), 0.68 (m, 1H), 0.60 (m, 1H), 0.47 (m, 1H), 0.39 (m, 1H). ¹³C NMR (100 MHz, DMSO-d₆): δ 170.6, 169.8, 149.5, 131.2, 129.9, 123.3, 119.4, 117.8, 117.6, 116.5, 116.1, 115.8, 113.5, 108.1, 107.9, 89.0, 69.0, 60.7, 57.0, 45.0, 34.6, 32.1, 29.7, 29.1, 23.8, 19.5, 8.4, 5.6, 5.1, 2.5. IR (diamond, cm⁻¹) vₘₐₓ: 3222, 1749, 1644, 1494, 1116, 1068, 748. HRMS: m/z calc. 450.2315 [M+H]⁺, obs. 450.2390 [M+H]⁺. The purity of the compound was checked by HPLC (Rt=6.50 min) and was found to be 95.14% pure.

### 17-Cyclopropylmethyl-3,14β-dihydroxy-4,5α-epoxy-6β-[(3′-pyrrolyl)acetamido]morphinan hydrochloride (10)

Compound 10 was synthesized as shown in the general procedure with 74% yield. ¹H NMR (400 MHz, DMSO-d₆): δ 10.55 (s, 1H), 9.34 (s, 1H), 8.82 (s, 1H), 8.16 (d, J=8 Hz, 1H), 6.71 (d, J=8 Hz, 1H), 6.63 (d, J=8 Hz, 1H), 6.61 (m, 1H), 6.15 (s, 1H), 5.90 (m, 1H), 5.82 (m, 1H), 4.58 (d, J=8 Hz, 1H), 3.83 (m, 1H), 3.39 (s, 2H), 3.3 (m, 1H), 3.04 (m, 3H), 2.84 (m, 1H), 2.42 (m, 2H), 1.72 (m, 2H), 1.51 (m, 1H), 1.43 (m, 1H), 1.33 (m, 2H), 1.07 (m, 1H), 0.67 (m, 1H), 0.58 (m, 1H), 0.50 (m, 1H), 0.40 (m, 1H). ¹³C NMR (100 MHz, DMSO-d₆): δ 170.8, 142.1, 141.2, 129.6, 120.5, 119.1, 117.8, 117.3, 115.8, 108.0, 89.9, 69.6, 61.6, 56.6, 50.6, 46.4, 45.5, 34.8, 29.2, 27.3, 23.6, 22.9, 5.6, 5.0, 2.5. IR (diamond, cm⁻¹) vₘₐₓ: 3062, 1656, 1315, 1127, 1033, 726. HRMS: m/z calc. 450.2315 [M+H]⁺, obs. 450.2369 [M+H]⁺. The purity of the compound was checked by HPLC (Rt=6.47 min) and was found to be 99.86% pure.

### 17-Cyclopropylmethyl-3,14β-dihydroxy-4,5α-epoxy-6β-[(3′-pyrrolyl)propanamido]morphinan hydrochloride (11)

Compound 11 was synthesized as shown in the general procedure with 41% yield. ¹H NMR (400 MHz, DMSO-d₆): δ 11.53 (s, 1H), 10.11 (s, 1H), 9.23 (s, 1H), 8.86 (s, 1H), 7.58 (d, J=8 Hz, 1H), 6.89 (m, 1H), 6.85 (m, 1H), 6.71 (d, J=8 Hz, 1H), 6.58 (d, J=8 Hz, 1H), 6.31 (s, 1H), 6.10 (m, 1H), 4.72 (d, J=4 Hz, 1H), 4.56 (m, 1H), 3.93 (d, J=4 Hz, 1H), 3.41 (s, 1H), 3.28 (m, 3H), 2.96 (m, 1H), 2.73 (m, 1H), 1.91 (m, 1H), 1.64 (m, 1H), 1.46 (m, 2H), 1.11 (m, 2H), 0.70 (m, 1H), 0.62 (m, 1H), 0.50 (m, 1H), 0.40 (m, 1H). ¹³C NMR (100 MHz, DMSO-d₆): δ 159.9, 146.0, 138.7, 128.7, 125.9, 122.0, 121.4, 119.1, 118.3, 110.8, 108.5, 87.5, 69.3, 61.0, 56.9, 45.3, 45.1, 45.1, 45.1, 30.2, 29.2, 23.4, 19.5, 8.4, 5.6, 5.1, 2.5. IR (diamond, cm⁻¹) vₘₐₓ: 3224, 1635, 1456, 1117, 1032, 944, 746. HRMS: m/z calc. 464.2471 [M+H]⁺, obs. 464.2522 [M+H]⁺. The purity of the compound was checked by HPLC (Rt=6.41 min) and was found to be 96.13% pure.

### 17-Cyclopropylmethyl-3,14β-dihydroxy-4,5α-epoxy-6β-[(3′-pyrrolyl)propanamido]morphinan hydrochloride (12)

Compound 12 was synthesized as shown in the general procedure with 72% yield. ¹H NMR (400 MHz, DMSO-d₆): δ 11.52 (s, 1H), 10.01 (s, 1H), 9.23 (s, 1H), 8.84 (s, 1H), 7.75 (d, J=8 Hz, 1H), 6.89 (m, 1H), 6.85 (m, 1H), 6.72 (d, J=8 Hz, 1H), 6.58 (d, J=8 Hz, 1H), 6.32 (s, 1H), 6.10 (m, 1H), 4.73 (m, 1H), 4.52 (d, J=4 Hz, 1H), 3.92 (d, J=8 Hz, 1H), 3.29 (m, 2H), 2.96 (m, 1H), 2.72 (m, 1H), 2.46 (m, 1H), 1.92 (m, 1H), 1.64 (m, 1H), 1.46 (m, 2H), 1.08 (m, 2H), 0.70 (m, 1H), 0.62 (m, 1H), 0.49 (m, 1H), 0.40 (m, 1H). ¹³C NMR (100 MHz, DMSO-d₆): δ 171.6, 142.1, 141.2, 129.6, 121.5, 120.5, 119.1, 117.8, 117.2, 114.5, 107.3, 89.9, 69.7, 61.6, 56.6, 50.5, 46.4, 45.5, 37.5, 29.2, 27.3, 23.6, 22.9, 22.7, 5.7, 5.0, 2.5. IR (diamond, cm⁻¹) vₘₐₓ: 3067, 1656, 1315, 1127, 748. HRMS: m/z calc. 464.2471 [M+H]⁺, obs. 464.2523 [M+H]⁺. The purity of the compound was checked by HPLC (RT=6.33 min) and was found to be 96.93% pure.

### 17-Cyclopropylmethyl-3,14β-dihydroxy-4,5α-epoxy-6α-[(2′-furanyl)carboxamido]morphinan hydrochloride (13)

Compound 13 was synthesized as shown in the general procedure with 87% yield. ¹H NMR (400 MHz, DMSO-d₆): δ 9.25 (s, 1H), 8.87 (s, 1H), 7.86 (m, 1H), 7.74 (d, J=8 Hz, 1H), 7.20 (m, 1H), 6.73 (d, J=8 Hz, 1H), 6.65 (m, 1H), 6.58 (d, J=8 Hz, 1H), 6.35 (s, 1H), 4.72 (d, J=4 Hz, 1H), 4.57 (m, 1H), 3.93 (m, 1H), 3.32 (s, 1H), 3.26 (m, 1H), 3.08 (m, 3H), 2.97 (m, 1H), 2.72 (m, 1H), 1.93 (m, 1H), 1.64 (m, 1H), 1.47 (m, 1H), 1.20 (t, J=8 Hz, 1H), 0.69 (m, 1H), 0.61 (m, 1H), 0.51 (m, 1H), 0.40 (m, 1H). ¹³C NMR (100 MHz, DMSO-d₆): δ 157.1, 147.4, 145.8, 145.1, 138.8, 128.6, 122.0, 119.2, 118.3, 113.9, 111.9, 87.2, 69.2, 64.8, 60.9, 57.0, 45.2, 30.1, 29.1, 23.4, 19.5, 15.1, 5.6, 5.1, 2.5. IR (diamond, cm⁻¹) vₘₐₓ: 3245, 1634, 1505, 1117, 1032, 727. HRMS: m/z calc. 437.1998 [M+H]⁺, obs. 437.2079 [M+H]⁺. The purity of the compound was checked by HPLC (Rt=7.72 min) and was found to be 100% pure.

### 17-Cyclopropylmethyl-3,14β-dihydroxy-4,5α-epoxy-6β-[(2′-furanyl)carboxamido]morphinan hydrochloride (14)

Compound 14 was synthesized as shown in the general procedure with 64% yield. ¹H NMR (400 MHz, DMSO-d₆): δ 9.30 (s, 1H), 8.86 (s, 1H), 8.57 (d, J=8 Hz, 1H), 7.85 (m, 1H), 7.13 (m, 1H), 6.73 (d, J=8 Hz, 1H), 6.66 (d, J=8 Hz, 1H), 6.64 (m, 1H), 6.18 (m, 1H), 4.84 (d, J=8 Hz, 1H), 3.38 (s, 1H), 3.64 (m, 1H), 3.08 (m, 3H), 2.87 (m, 1H), 2.71 (m, 1H), 2.45 (m, 1H), 1.91 (m, 1H), 1.77 (m, 1H), 1.54 (m, 1H), 1.42 (m, 2H), 1.20 (t, J=8 Hz, 1H), 1.08 (m, 1H), 0.69 (m, 1H), 0.60 (m, 1H), 0.52 (m, 1H), 0.42 (m, 1H). ¹³C NMR (100 MHz, DMSO-d₆): δ 157.3, 147.8, 144.9, 142.0, 141.3, 129.6, 120.5, 119.2, 117.8, 113.3, 111.8, 89.6, 69.6, 61.7, 56.6, 50.4, 45.5, 29.4, 27.2, 23.7, 23.0, 8.4, 5.7, 5.0, 2.6. IR (diamond, cm⁻¹) vₘₐₓ: 3010, 1644, 1503, 1126, 748. HRMS: m/z calc. 437.1998 [M+H]⁺, obs. 437.2087 [M+H]⁺. The purity of the compound was checked by HPLC (Rt=6.31 min) and was found to be 98.05% pure.

### 17-Cyclopropylmethyl-3,14β-dihydroxy-4,5α-epoxy-6α-[(2′-furanyl)acetamido]morphinan hydrochloride (15)

Compound 15 was synthesized as shown in the general procedure with 74% yield. ¹H NMR (400 MHz, DMSO-d₆): δ 10.54 (s, 1H), 9.25 (s, 1H), 8.87 (s, 1H), 7.78 (d, J=8 Hz, 1H), 6.73 (d, J=8 Hz, 1H), 6.60 (m, 1H), 6.55 (d, J=8 Hz, 1H), 6.30 (s, 1H), 5.89 (m, 1H), 5.82 (m, 1H), 4.58 (d, J=4 Hz, 1H), 4.38 (m, 1H), 3.93 (m, 1H), 3.44 (s, 1H), 3.38 (m, 1H), 3.27 (m, 2H), 3.04 (m, 2H), 2.94 (m, 1H), 2.70 (m, 1H), 2.43 (m, 1H), 1.87 (m, 1H), 1.59 (m, 1H), 1.39 (m, 2H), 1.07 (m, 1H), 0.93 (m, 1H), 0.67 (m, 1H), 0.60 (m, 1H), 0.48 (m, 1H), 0.39 (m, 1H). ¹³C NMR (100 MHz, DMSO-d₆): δ 169.0, 145.9, 138.8, 128.7, 125.4, 122.0, 119.0, 118.2, 116.6, 107.1, 105.8, 87.4, 69.3, 64.8, 60.9, 56.9, 48.5, 45.1, 34.8, 30.1, 29.1, 23.4, 19.7, 5.6, 5.1, 2.5. IR (diamond, cm⁻¹) vₘₐₓ: 3055, 1660, 1563, 1119, 745. HRMS: m/z calc. 451.2155 [M+H]⁺, obs. 451.2207 [M+H]⁺. The purity of the compound was checked by HPLC (Rt=4.84 min) and was found to be 96.72% pure.

### 17-Cyclopropylmethyl-3,14β-dihydroxy-4,5α-epoxy-6β-[(2′-furanyl)acetamido]morphinan hydrochloride (16)

Compound 16 was synthesized as shown in the general procedure with 53% yield. ¹H NMR (400 MHz, DMSO-d₆): δ 9.34 (s, 1H), 8.83 (s, 1H), 8.34 (d, J=8 Hz, 1H), 7.55 (s, 1H), 6.72 (d, J=8 Hz, 1H), 6.64 (d, J=8 Hz, 1H), 6.39 (m, 1H), 6.21 (m, 2H), 4.59 (d, J=8 Hz, 1H), 3.85 (d, J=4 Hz, 1H), 3.51 (s, 2H), 3.05 (m, 3H), 2.85 (m, 1H), 2.73 (m, 1H), 2.42 (m, 2H), 1.71 (m, 2H), 1.54 (m, 1H), 1.44 (m, 1H), 1.34 (m, 1H), 1.13 (m, 2H), 0.69 (m, 1H), 0.59 (m, 1H), 0.51 (m, 1H), 0.41 (m, 1H). ¹³C NMR (100 MHz, DMSO-d₆): δ 167.3, 149.7, 142.0, 141.8, 141.2, 129.5, 120.5, 119.2, 117.9, 110.4, 107.3, 89.7, 69.6, 61.6, 56.6, 50.9, 46.4, 45.5, 35.3, 29.2, 27.3, 23.5, 22.9, 5.6, 5.0, 2.5. IR (diamond, cm⁻¹) vₘₐₓ: 3005, 1658, 1466, 1128, 747. HRMS: m/z calc. 451.2155 [M+H]⁺, obs. 451.2225 [M+H]⁺. The purity of the compound was checked by HPLC (Rt=7.45 min) and was found to be 97.44% pure.

### 17-Cyclopropylmethyl-3,14β-dihydroxy-4,5α-epoxy-6α-[(2′-furanyl)propanamido]morphinan hydrochloride (17)

Compound 17 was synthesized as shown in the general procedure with yield 65%. ¹H NMR (400 MHz, DMSO-d₆): δ 9.87 (s, 1H), 9.17 (s, 1H), 8.82 (s, 1H), 7.74 (d, J=8 Hz, 1H), 7.49 (s, 1H), 6.71 (d, J=8 Hz, 1H), 6.56 (d, J=8 Hz, 1H), 6.34 (m, 1H), 6.22 (s, 1H), 6.11 (m, 1H), 4.59 (d, J=4 Hz, 1H), 4.41 (m, 1H), 3.88 (d, J=7 Hz, 1H), 3.34 (d, J=16 Hz, 1H), 3.27 (m, 1H), 3.06 (m, 6H), 2.93 (m, 1H), 2.84 (m, 2H), 2.70 (m, 1H), 2.46 (m, 1H), 1.84 (m, 1H), 1.60 (m, 1H), 1.37 (m, 2H), 1.07 (m, 1H), 0.93 (m, 1H), 0.69 (m, 1H), 0.61 (m, 1H), 0.46 (m, 1H), 0.39 (m, 1H). ¹³C NMR (100 MHz, DMSO-d₆): δ 170.3, 154.7, 145.9, 141.2, 138.7, 128.7, 122.0, 119.0, 118.1, 110.3, 104.9, 87.4, 69.3, 61.0, 56.9, 45.4, 45.1, 45.1, 44.9, 33.2, 29.1, 23.4, 19.6, 8.4, 5.6, 5.0, 2.5. IR (diamond, cm⁻¹) vₘₐₓ: 3188, 2981, 2947, 1635, 1506, 1455, 1116, 1033, 744. HRMS: m/z calc. 465.2311 [M+H]⁺, 487.2209 [M+Na]⁺, obs. 465.2404 [M+H]⁺, 487.2220 [M+Na]⁺. The purity of the compound was checked by HPLC (Rt=6.25 min) and was found to be 97.59% pure.

### 17-Cyclopropylmethyl-3,14β-dihydroxy-4,5α-epoxy-6β-[(2′-furanyl)propanamido]morphinan hydrochloride (18)

Compound 18 was synthesized as shown in the general procedure with 72% yield. ¹H NMR (400 MHz, DMSO-d₆): δ 9.31 (s, 1H), 8.82 (s, 1H), 8.19 (m, 1H), 7.50 (m, 1H), 6.72 (d, J=8 Hz, 1H), 6.64 (d, J=8 Hz, 1H), 6.35 (m, 1H), 6.16 (m, 1H), 6.10 (m, 1H), 4.55 (d, J=8 Hz, 1H), 3.83 (s, 1H), 3.40 (m, 2H), 3.06 (m, 2H), 2.83 (m, 3H), 2.41 (m, 4H), 1.70 (m, 2H), 1.47 (m, 2H), 1.33 (m, 1H), 1.12 (m, 1H), 0.70 (m, 1H), 0.59 (m, 1H), 0.51 (m, 1H), 0.41 (m, 1H). ¹³C NMR (100 MHz, DMSO-d₆): δ 170.4, 154.6, 142.0, 141.2, 141.2, 129.5, 120.5, 119.2, 117.8, 110.3, 105.0, 89.8, 69.6, 61.6, 56.6, 55.9, 50.6, 46.4, 45.5, 33.7, 29.2, 27.3, 23.4, 22.9, 5.6, 5.0, 2.5. IR (diamond, cm⁻¹) vₘₐₓ: 3161, 1639, 1538, 1298, 1124, 1032, 748. HRMS: m/z calc. 465.2311 [M+H]⁺, obs. 465.2374 [M+H]⁺. The purity of the compound was checked by HPLC (Rt=7.70 min) and was found to be 99.80% pure.

### 17-Cyclopropylmethyl-3,14β-dihydroxy-4,5α-epoxy-6α-[(3′-furanyl)carboxamido]morphinan hydrochloride (19)

Compound 19 was synthesized as shown in the general procedure with 34% yield. ¹H NMR (400 MHz, DMSO-d₆): δ 9.22 (s, 1H), 8.85 (s, 1H), 8.28 (s, 1H), 7.88-7.89 (m, 1H), 7.74 (m, 1H), 6.93 (s, 1H), 6.71 (d, J=8 Hz, 1H), 6.57 (d, J=8 Hz, 1H), 6.31 (s, 1H), 4.72 (d, J=4 Hz, 1H), 4.56 (m, 1H), 3.91 (m, 1H), 3.28 (m, 2H), 3.27 (m, 3H), 2.95 (m, 1H), 2.71 (m, 2H), 1.92 (m, 1H), 1.63 (m, 1H), 1.45 (m, 2H), 1.04 (m, 1H), 0.70 (m, 1H), 0.61 (m, 1H), 0.49 (m, 1H), 0.40 (m, 1H). ¹³C NMR (100 MHz, DMSO-d₆): δ 161.1, 146.0, 145.3, 143.8, 138.7, 128.6, 122.5, 122.0, 119.0, 109.2, 87.1, 69.3, 56.9, 45.4, 45.1, 30.2, 29.1, 23.4, 19.2, 8.4, 5.6, 5.1, 2.5. IR (diamond, cm⁻¹) vₘₐₓ: 3034, 1640, 1504, 1116, 1030, 946. HRMS: m/z calc. 437.1998 [M+H]⁺, obs. 437.2053 [M+H]⁺. The purity of the compound was checked by HPLC (Rt=4.76 min) and was found to be 97.49% pure.

### 17-Cyclopropylmethyl-3,14β-dihydroxy-4,5α-epoxy-6β-[(3′-furanyl)carboxamido]morphinan hydrochloride (20)

Compound 20 was synthesized as shown in the general procedure with 67% yield. ¹H NMR (400 MHz, DMSO-d₆): δ 10.54 (s, 1H), 9.25 (s, 1H), 8.86 (s, 1H), 7.77 (d, J=8 Hz, 1H), 6.72 (d, J=8 Hz, 1H), 6.60 (m, 1H), 6.56 (d, J=8 Hz, 1H), 6.29 (s, 1H), 5.90 (m, 1H), 5.82 (m, 1H), 4.58 (d, J=3.84 Hz, 1H), 4.39 (m, 1H), 3.91 (d, J=4 Hz, 1H), 3.31 (s, 1H), 3.24 (m, 1H), 3.03 (m, 2H), 2.95 (m, 1H), 2.70 (m, 1H), 2.44 (m, 1H), 1.86 (m, 1H), 1.61 (m, 1H), 1.39 (m, 2H), 1.07 (m, 1H), 0.94 (m, 1H), 0.69 (m, 1H), 0.60 (m, 1H), 0.49 (m, 1H), 0.39 (m, 1H). ¹³C NMR (100 MHz, DMSO-d₆): δ 169.0, 145.9, 138.8, 128.7, 125.3, 122.0, 119.0, 118.2, 116.6, 107.1, 105.8, 87.4, 69.3, 60.9, 57.5, 56.9, 45.1, 34.8, 30.1, 29.1, 23.4, 19.6, 5.6, 5.1, 2.5. IR (diamond, cm⁻¹) vₘₐₓ: 3333, 1647, 1456, 1119, 870, 748. HRMS: m/z calc. 437.1998 [M+H]⁺, 459.1896 [M+Na]⁺, obs. 437.2091 [M+H]⁺, 459.1911 [M+Na]⁺. The purity of the compound was checked by HPLC (Rt=6.41 min) and was found to be 97.12% pure.

### 17-Cyclopropylmetyl-3,14β-dihydroxy-4,5α-epoxy-6α-[(3′-furanyl)acetamido]morphinan hydrochloride (21)

Compound 21 was synthesized as shown in the general procedure with 73% yield. ¹H NMR (400 MHz, DMSO-d₆): δ 10.55 (s, 1H), 9.26 (s, 1H), 8.85 (s, 1H), 7.78 (d, J=8 Hz, 1H), 6.73 (d, J=8 Hz, 1H), 6.60 (m, 1H), 6.56 (d, J=8 Hz, 1H), 6.29 (s, 1H), 5.91-5.89 (m, 1H), 5.82 (m, 1H), 4.59 (d, J=4 Hz, 1H), 4.39 (m, 1H), 3.91 (d, J=8 Hz, 1H), 3.34 (d, J=20 Hz, 1H), 3.24 (m, 1H), 3.03 (m, 2H), 2.94 (m, 1H), 2.70 (m, 1H), 2.43 (m, 1H), 1.86 (m, 1H), 1.59 (m, 1H), 1.41 (m, 2H), 1.07 (m, 1H), 0.98 (m, 1H), 0.70 (m, 1H), 0.60 (m, 1H), 0.49 (m, 1H), 0.38 (m, 1H). ¹³C NMR (100 MHz, DMSO-d₆): δ 169.0, 145.9, 138.8, 128.7, 125.3, 122.0, 119.0, 118.2, 116.6, 107.1, 105.8, 87.4, 69.3, 60.9, 56.9, 48.5, 45.1, 35.1, 30.0, 29.1, 23.4, 19.6, 5.6, 5.1, 2.5. IR (diamond, cm⁻¹) vₘₐₓ: 3231, 1640, 1319, 1117, 1032, 725. HRMS: m/z calc. 451.2155 [M+H]⁺, obs. 451.2209 [M+H]⁺. The purity of the compound was checked by HPLC (Rt=4.80 min) and was found to be 96.01% pure.

### 17-Cyclopropylmetyl-3,14β-dihydroxy-4,5α-epoxy-6β-[(3′-furanyl)acetamido]morphinan hydrochloride (22)

Compound 22 was synthesized as shown in the general procedure with 46% yield. ¹H NMR (400 MHz, DMSO-d₆): δ 9.31 (s, 1H), 8.81 (s, 1H), 8.24 (d, J=8 Hz, 1H), 7.58 (m, 1H), 7.51 (s, 1H), 6.72 (d, J=8 Hz, 1H), 6.64 (d, J=8 Hz, 1H), 6.42 (m, 1H), 6.13 (m, 1H), 4.58 (d, J=8 Hz, 1H), 3.83 (m, 1H), 3.25 (m, 2H), 3.05 (m, 2H), 2.84 (m, 1H), 2.42 (m, 2H), 1.71 (m, 2H), 1.52 (m, 1H), 1.44 (m, 1H), 1.34 (m, 1H), 1.07 (m, 1H), 0.68 (m, 1H), 0.60 (m, 1H), 0.51 (m, 1H), 0.41 (m, 1H). ¹³C NMR (100 MHz, DMSO-d₆): δ 169.4, 142.8, 141.9, 141.0, 139.9, 129.8, 120.7, 119.2, 119.1, 117.9, 111.5, 89.6, 69.6, 61.8, 56.4, 50.7, 46.4, 45.5, 32.0, 29.3, 27.2, 23.5, 22.9, 5.6, 5.0, 2.5. IR (diamond, cm⁻¹) vₘₐₓ: 3057, 1659, 1500, 1129, 1011, 771. HRMS: m/z calc. 451.2155 [M+H]⁺, obs. 451.2241 [M+H]⁺. The purity of the compound was checked by HPLC (Rt=7.50 min) and was found to be 98.73% pure.

### 17-Cyclopropylmetyl-3,14β-dihydroxy-4,5α-epoxy-6α-[(3′-furanyl)propanamido]morphinan hydrochloride (23)

Compound 23 was synthesized as shown in the general procedure with 51% yield. ¹H NMR (400 MHz, DMSO-d₆): δ 9.15 (s, 1H), 8.84 (s, 1H), 7.69 (d, J=8 Hz, 1H), 7.54 (m, 1H), 7.44 (m, 1H), 6.72 (d, J=8 Hz, 1H), 6.56 (d, J=8 Hz, 1H), 6.39 (m, 1H), 6.24 (s, 1H), 4.58 (d, J=4 Hz, 1H), 4.43-4.37 (m, 1H), 3.90 (d, J=4 Hz, 1H), 3.26 (m, 1H), 3.04 (m, 2H), 2.95 (m, 1H), 2.71 (m, 1H), 2.63 (t, 2H), 2.45 (d, J=4 Hz, 1H), 2.40 (t, 2H), 1.85 (m, 1H), 1.61 (m, 1H), 1.37 (m, 2H), 1.05 (m, 1H), 0.91 (m, 1H), 0.68 (m, 1H), 0.61 (m, 1H), 0.48 (m, 1H), 0.39 (m, 1H). ¹³C NMR (100 MHz, DMSO-d₆): δ 170.9, 145.9, 142.9, 138.8, 138.7, 128.7, 124.0, 122.0, 119.0, 118.2, 111.1, 87.5, 69.3, 64.8, 61.0, 56.9, 45.1, 44.8, 35.3, 30.1, 29.1, 23.4, 20.4, 19.6, 15.1, 5.6, 5.1, 2.5. IR (diamond, cm⁻¹) vₘₐₓ: 3229, 1651, 1452, 1117, 1068, 749. HRMS: m/z calc. 465.2311 [M+H]⁺, obs. 465.2403 [M+H]⁺. The purity of the compound was checked by HPLC (Rt=4.88 min) and was found to be 100% pure.

### 17-Cyclopropylmetyl-3,14β-dihydroxy-4,5α-epoxy-6β-[(3′-furanyl)propanamido]morphinan hydrochloride (24)

Compound 24 was synthesized as shown in the general procedure with 57% yield. ¹H NMR (400 MHz, DMSO-d₆): δ 9.22 (s, 1H), 8.84 (s, 1H), 8.28 (s, 1H), 7.89-7.87 (m, 1H), 7.74 (t, J=1.64 Hz, 1H), 6.93 (s, 1H), 6.72 (d, J=8 Hz, 1H), 6.57 (d, J=8 Hz, 1H), 6.30 (m, 1H), 4.72 (d, J=3.84 Hz, 1H), 4.55 (m, 1H), 3.90 (m, 1H), 2.94 (m, 1H), 2.72 (m, 2H), 1.91 (m, 1H), 1.63 (m, 1H), 1.46 (m, 2H), 1.11 (m, 3H), 0.69 (m, 1H), 0.61 (m, 1H), 0.49 (m, 1H), 0.40 (m, 1H). ¹³C NMR (100 MHz, DMSO-d₆): δ 171.0, 142.8, 142.1, 141.2, 138.9, 129.6, 123.9, 120.5, 119.1, 117.8, 111.2, 89.8, 69.6, 61.6, 56.6, 50.5, 46.4, 45.5, 35.9, 29.3, 27.3, 23.6, 22.9, 20.3, 5.6, 5.0, 2.5. IR (diamond, cm⁻¹) vₘₐₓ: 3059, 1634, 1500, 1124, 873, 748. HRMS: m/z calc. 465.2311 [M+H]⁺, 487.2209 [M+Na]⁺, obs. 465.2400 [M+H]⁺, 487.2225 [M+Na]⁺. The purity of the compound was checked by HPLC (Rt=6.43 min) and was found to be 97.61% pure.

### 17-Cyclopropylmethyl-3,14β-dihydroxy-4,5α-epoxy-6α-(2′-thienylcarboxamido)morphinan hydrochloride(25)

Compound 25 was synthesized as shown in the general procedure with 82.57% yield. ¹H NMR (400 MHz, DMSO-d₆) δ 9.30 (s, 1H, exchangeable), 8.82 (s, 1H, exchangeable), 8.17 (d, J=7.7 Hz, 1H, exchangeable), 7.88 (d, J=3.6 Hz, 1H), 7.77 (d, J=5.0 Hz, 1H), 7.17 (t, J=4.3 Hz, 1H), 6.71 (d, J=8.2 Hz, 1H), 6.59 (d, J=8.1 Hz, 1H), 6.25 (s, 1H, exchangeable), 4.76 (d, J=3.7 Hz, 1H), 4.55 (s, 1H), 3.07 (m, 3H), 2.93 (s, 2H), 2.71 (d, J=21.5 Hz, 1H), 1.89 (d, J=15.3 Hz, 1H), 1.64 (d, J=13.5 Hz, 1H), 1.48 (m, 2H), 1.19 (m, 2H), 1.04 (d, J=6.1 Hz, 1H), 0.70 (m, 1H), 0.62 (m, 1H), 0.47 (m, 1H), 0.40 (m, 1H). ¹³C NMR (100 MHz, DMSO-d₆) δ 160.8, 146.4, 145.9, 145.4, 139.5, 138.7, 131.0, 128.5, 127.8, 119.1, 87.1, 69.2, 65.6, 61.1, 57.0, 47.5, 46.0, 45.2, 29.2, 23.3, 20.9, 15.1, 5.6, 5.1, 2.5. IR (diamond, cm⁻¹) vₘₐₓ: 3069, 1621, 1537, 1456, 1316, 1031, 745. HRMS calc. 453.1803 [M+H]⁺, obs. 453.1854 [M+H]⁺. Mp 233.5-235.8° C. The purity of the compound was checked by HPLC (Rt=6.49 min) and was found to be 97.47% pure.

### 17-Cyclopropylmethyl-3,14β-dihydroxy-4,5α-epoxy-6β-(2′-thienylcarboxamido)morphinan hydrochloride(26)

Compound 26 was synthesized as shown in the general procedure with 30.55% yield. ¹H NMR (400 MHz, DMSO-d₆): δ. 9.36 (s, 1H, exchangeable), 8.86 (s, 1H, exchangeable), 8.72 (d, J=8.3 Hz, 1H, exchangeable), 7.83 (dd, J=3.7, 1.1 Hz, 1H), 7.78 (dd, J=5.1, 1.1 Hz, 1H), 7.18 (dd, J=5.0, 3.7 Hz, 1H), 6.73 (d, J=8.1 Hz, 1H), 6.67 (d, J=8.2 Hz, 1H), 6.18 (s, 1H, exchangeable), 4.80 (d, J=7.8 Hz, 1H), 3.87 (d, J=5.6 Hz, 1H), 3.69-3.57 (m, 1H), 3.12 (d, J=6.0 Hz, 1H), 3.10-3.01 (m, 1H), 2.85 (t, J J=9.8 Hz, 1H), 2.45 (dd, J=10.4, 3.3 Hz, 2H), 1.89 (m, 1H), 1.77 (m, 1H), 1.59 (m, 1H), 1.44 (m, 2H), 1.09 (m, 1H), 0.69 (m, 1H), 0.60 (m, 1H), 0.52 (m, 1H), 0.42 (m, 1H). ¹³C NMR (100 MHz, DMSO-d₆) δ: 160.7, 141.9, 141.1, 139.9, 139.8, 130.9, 129.5, 128.0, 127.9, 120.5, 119.3, 117.8, 89.6, 69.5, 61.6, 56.6, 50.9, 46.4, 45.5, 29.2, 27.2, 23.7, 22.9, 5.6, 5.0, 2.5. IR (diamond, cm⁻¹) vₘₐₓ: 3074, 1646, 1543, 1462, 1319, 1021, 746. HRMS: m/z calc. 453.1803 [M+H]⁺, 475.1701 [M+Na]⁺, 927.3588 [2M+Na], obs. 453.1845 [M+H]⁺, 475.1646 [M+Na]⁺, 927.3049 [2M+Na]⁺. Mp. 289-292.6° C. The purity of the compound was checked by HPLC (Rt=6.42 min) and was found to be 99.82% pure.

### 17-Cyclopropylmethyl-3,14β-dihydroxy-4,5α-epoxy-6α-(2′-thienylacetamido)morphinan hydrochloride(27)

Compound 27 was synthesized as shown in the general procedure with 40.17% yield. ¹H NMR (400 MHz, DMSO-d₆): δ. 9.22 (s, 1H, exchangeable), 8.81 (s, 1H, exchangeable), 8.01 (d, J=7.9 Hz, 1H, exchangeable), 7.36 (dd, J=5.1, 1.4 Hz, 1H), 6.70 (m, 2H), 6.72 (d, J=8.1 Hz, 1H), 6.57 (d, J=8.1 Hz, 1H), 6.19 (s, 1H, exchangeable), 4.60 (s, 1H), 4.40 (s, 1H), 3.87 (s, 1H), 3.73 (d, J=1.0 Hz, 2H), 3.18 (d, J=4.5 Hz, 1H), 3.03 (s, 2H), 2.92 (d, J=13.6 Hz, 1H), 2.71 (d, J=24.2 Hz, 1H), 2.45 (d, J=18.2 Hz, 1H), 1.83 (s, 1H), 1.62 (d, J=13.6 Hz, 1H), 1.42 (dd, J=14.8, 9.8 Hz, 2H), 1.25 (s, 1H), 1.00 (m, 2H), 0.68 (m, 1H), 0.61 (m, 1H), 0.47 (m, 1H), 0.40 (m, 1H). ¹³C NMR (100 MHz, DMSO-d₆) δ: 168.6, 145.9, 138.7, 137.6, 128.6, 126.4, 125.88, 124.7, 119.0, 118.1, 87.3, 69.2, 60.9, 56.9, 48.5, 45.1, 36.2, 30.1, 29.1, 23.4, 19.5, 5.6, 5.1, 2.5. IR (diamond, cm⁻¹) vₘₐₓ: 3134, 1634, 1543, 1457, 1321, 1033, 746. HRMS: m/z calc. 467.1960 [M+H]⁺, 489.1858 [M+Na]⁺, obs. 467.2001 [M+H]⁺, 489.1813 [M+Na]⁺. Mp. 198.5-200.1° C. The purity of the compound was checked by HPLC (Rt=6.68 min) and was found to be 97.70% pure.

### 17-Cyclopropylmethyl-3,14β-dihydroxy-4,5α-epoxy-6β-(2′-thienylacetamido)morphinan hydrochloride (28)

Compound 28 was synthesized as shown in the general procedure with 86.61% yield. ¹H NMR (400 MHz, DMSO-d₆): δ. 9.34 (s, 1H, exchangeable), 8.81 (s, 1H, exchangeable), 8.41 (d, J=7.8 Hz, 1H, exchangeable), 7.36 (dd, J=5.1, 1.3 Hz, 1H), 6.96 (dd, J=5.2, 3.4 Hz, 1H), 6.92 (dd, J=3.4, 1.3 Hz, 1H), 6.72 (d, J=8.1 Hz, 1H), 6.65 (d, J=8.2 Hz, 1H), 6.13 (s, 1H, exchangeable), 4.58 (d, J=7.8 Hz, 1H), 3.82 (m, 1H), 3.66 (m, 2H), 3.08 (dq, J=7.3, 2.6 Hz, 1H), 3.03 (dt, J=10.4, 4.5 Hz, 1H), 2.85 (m, 1H), 2.41 (m, 1H), 1.71 (td, J=14.9, 14.2, 3.0 Hz, 2H), 1.54 (m, 1H), 1.45 (d, J=10.3 Hz, 1H), 1.34 (m, 1H), 1.07 (m, 1H), 0.68 (m, 1H), 0.59 (m, 1H), 0.50 (m, 1H), 0.41 (m, 1H). ¹³C NMR (100 MHz, DMSO-d₆) δ: 168.7, 142.0, 141.2, 137.4, 129.5, 126.5, 125.9, 124.8, 120.5, 119.2, 117.9, 89.7, 69.6, 61.6, 56.6, 50.9, 46.4, 45.5, 36.7, 29.2, 27.3, 23.4, 22.9, 5.6, 5.0, 2.5. IR (diamond, cm⁻¹) vₘₐₓ: 3003, 1658, 1553, 1466, 1314, 1031, 749. HRMS: m/z calc. 467.1960 [M+H]⁺, 489.1858 [M+Na]⁺, obs. 467.2032 [M+H]⁺, 489.1842 [M+Na]⁺. Mp. 210-212° C. The purity of the compound was checked by HPLC (Rt=6.53 min) and was found to be 96.39% pure.

### 17-Cyclopropylmethyl-3,14β-dihydroxy-4,5α-epoxy-6α-[3′-(thiophen-2″yl)propanamido]morphinan hydrochloride (29)

Compound 29 was synthesized as shown in the general procedure with 72.17% yield. ¹H NMR (400 MHz, DMSO-d₆): δ. 9.20 (s, 1H, exchangeable), 8.84 (s, 1H, exchangeable), 7.76 (d, J=8.1 Hz, 1H, exchangeable), 7.30 (d, J=5.1 Hz, 1H), 6.93 (dd, J=5.1, 3.4 Hz, 1H), 6.88 (d, J=3.4 Hz, 1H), 6.72 (d, J=8.1 Hz, 1H), 6.56 (d, J=8.1 Hz, 1H), 6.25 (s, 1H, exchangeable), 4.59 (d, J=4.1 Hz, 1H), 4.43 (tt, J=8.4, 4.1 Hz, 1H), 3.89 (d, J=7.0 Hz, 1H), 3.28 (m, 1H), 3.05 (m, 4H), 2.94 (m, 1H), 2.71 (m, 1H), 2.44 (dd, J=13.5, 4.9 Hz, 1H), 1.85 (dt, J=15.4, 9.4 Hz, 1H), 1.62 (dd, J=13.0, 3.5 Hz, 1H), 1.41 (dd, J=14.9, 9.5 Hz, 2H), 1.27 (t, J=7.2 Hz, 1H), 1.04 (m, 1H), 0.93 (tt, J=13.4, 8.2 Hz, 1H), 0.69 (m, 1H), 0.62 (m, 1H), 0.48 (m, 1H), 0.40 (m, 1H). ¹³C NMR (100 MHz, DMSO-d₆) δ: 170.3, 145.9, 143.7, 138.7, 128.6, 126.7, 124.4, 123.6, 122.0, 119.0, 118.1, 87.4, 69.3, 64.8, 61.0, 56.9, 45.1, 44.8, 36.8, 30.1, 29.1, 25.2, 23.4, 19.6, 5.6, 5.1, 2.5. IR (diamond, cm⁻¹) vₘₐₓ: 3065, 1644, 1587, 1456, 1319, 1032, 746. HRMS: m/z calc. 481.2116 [M+H]⁺, 503.2014 [M+Na]⁺, obs. 481.2159 [M+H]⁺, 503.1978 [M+Na]⁺. Mp. 173.4-175.6° C. The purity of the compound was checked by HPLC (Rt=6.71 min) and was found to be 98.46% pure.

### 17-Cyclopropylmethyl-3,14β-dihydroxy-4,5α-epoxy-6β-[3′-(thiophen-2 yl)propanamido]morphinan hydrochloride (30)

Compound 30 was synthesized as shown in the general procedure with 53.44% yield. ¹H NMR (400 MHz, DMSO-d₆): δ. 9.34 (s, 1H, exchangeable), 8.80 (s, 1H, exchangeable), 8.18 (d, J=7.9 Hz, 1H, exchangeable), 7.30 (dd, J=5.1, 1.3 Hz, 1H), 6.93 (dd, J=5.1, 3.4 Hz, 1H), 6.85 (m, 1H), 6.72 (d, J=8.1 Hz, 1H), 6.65 (d, J=8.2 Hz, 1H), 6.10 (s, 1H, exchangeable), is 4.54 (d, J=7.9 Hz, 1H), 3.81 (d, J=5.6 Hz, 1H), 3.03 (td, J=7.3, 3.3 Hz, 4H), 2.82 (m, 2H), 2.75 (dd, J=5.1, 2.8 Hz, 1H), 2.66 (m, 1H), 2.43 (m, 2H), 1.68 (d, J=12.7 Hz, 2H), 1.53 (d, J=8.4 Hz, 1H), 1.44 (d, J=11.0 Hz, 1H), 1.35 (m, 1H), 1.26 (t, J=5.9 Hz, 1H), 1.07 (m, 1H), 0.69 (d, J=5.6 Hz, 1H), 0.59 (m, 1H), 0.55 (m, 1H), 0.41 (m, 1H). ¹³C NMR (100 MHz, DMSO-d₆) δ: 170.5, 143.5, 142.0, 141.2, 129.5, 126.8, 124.5, 123.6, 120.5, 119.2, 117.8, 89.8, 69.6, 61.6, 56.6, 50.6, 46.4, 45.5, 37.3, 29.2, 27.2, 25.1, 23.5, 22.9, 5.6, 5.0, 2.5. IR (diamond, cm⁻¹) vₘₐₓ: 3053, 1644, 1532, 1455, 1298, 1061, 749. HRMS: m/z calc. 481.2116 [M+H]⁺, obs. 481.2166 [M+H]⁺. Mp. 207.4-209.8° C. The purity of the compound was checked by HPLC (Rt=6.74 min) and was found to be 97.48% pure.

### 17-Cyclopropylmethyl-3,14β-dihydroxy-4,5α-epoxy-6α-(3′-thienylcarboxamido)morphinan hydrochloride (31)

Compound 31 was synthesized as shown in the general procedure with 32.40% yield. ¹H NMR (400 MHz, DMSO-d₆): δ. 9.20 (s, 1H, exchangeable), 8.81 (s, 1H, exchangeable), 8.22 (dd, J=2.9, 1.3 Hz, 1H), 7.94 (d, J=7.5 Hz, 1H, exchangeable), 7.61 (dd, J=5.0, 2.9 Hz, 1H), 7.55 (dd, J=5.0, 1.3 Hz, 1H), 6.71 (d, J=8.1 Hz, 1H), 6.59 (d, J=8.1 Hz, 1H), 6.22 (s, 1H, exchangeable), 4.77 (d, J=3.9 Hz, 1H), 4.61-4.51 (m, 1H), 3.88 (d, J=7.1 Hz, 2H), 3.11 (d, J=6.6 Hz, 1H), 3.05 (m, 1H), 2.94 (m, 1H), 2.68 (d, J=2.3 Hz, 1H), 2.34 (d, J=2.2 Hz, 1H), 1.89 (m, 1H), 1.66 (d, J=13.3 Hz, 1H), 1.48 (m, 2H), 1.18 (d, J=8.0 Hz, 1H), 1.05 (m, 2H), 0.70 (m, 1H), 0.63 (m, 1H), 0.48 (m, 1H), 0.42 (m, 1H). ¹³C NMR (100 MHz, DMSO-d₆) δ: 161.6, 146.0, 138.7, 137.5, 128.9, 128.6, 127.1, 126.5, 122.0, 119.0, 118.2, 87.1, 69.3, 64.8, 61.0, 56.9, 46.4, 45.6, 45.1, 29.2, 23.4, 19.2, 13.6, 5.6, 5.1, 2.5. IR (diamond, cm⁻¹) vₘₐₓ: 3069, 1621, 1537, 1456, 1316, 1031, 745. HRMS: m/z calc. 453.1803 [M+H]⁺, obs. 453.1858 [M+H]⁺. Mp. 198.3-200.3° C. The purity of the compound was checked by HPLC (Rt=5.65 min) and was found to be 96.98% pure.

### 17-Cyclopropylmethyl-3,14β-dihydroxy-4,5α-epoxy-6β-(3′-thienylcarboxamido)morphinan hydrochloride (32)

Compound 32 was synthesized as shown in the general procedure with 67.60% yield. ¹H NMR (400 MHz, DMSO-d₆): δ. 9.31 (s, 1H, exchangeable), 8.83 (s, 1H, exchangeable), 8.49 (d, J=8.0 Hz, 1H, exchangeable), 8.16 (dd, J=3.0, 1.3 Hz, 1H), 7.61 (dd, J=5.0, 2.9 Hz, 1H), 7.53 (dd, J=5.0, 1.3 Hz, 1H), 6.73 (d, J=8.2 Hz, 1H), 6.67 (d, J=8.2 Hz, 1H), 6.12 (s, 1H, exchangeable), 4.79 (d, J=7.8 Hz, 1H), 3.85 (d, J=5.6 Hz, 1H), 3.66 (dt, J=12.9, 6.3 Hz, 1H), 3.12 (d, J=5.9 Hz, 1H), 3.05 (m, 1H), 2.99 (d, J=8.9 Hz, 1H), 2.85 (t, J=9.4 Hz, 1H), 2.45 (m, 2H), 1.87 (q, J=13.0 Hz, 1H), 1.76 (d, J=13.5 Hz, 1H), 1.62 (t, J=15.4 Hz, 1H), 1.45 (m, 2H), 1.26 (t, J=7.0 Hz, 1H), 1.09 (d, J=8.9 Hz, 1H), 0.70 (m, 1H), 0.61 (m, 1H), 0.52 (m, 1H), 0.43 (m, 1H). ¹³C NMR (100 MHz, DMSO-d₆) δ: 161.5, 142.1, 141.3, 137.7, 129.6, 128.7, 126.7, 120.5, 119.2, 117.9, 89.8, 69.7, 61.8, 56.7, 50.8, 46.4, 45.5, 29.3, 27.3, 23.0, 5.6, 5.0, 2.6. IR (diamond, cm⁻¹) vₘₐₓ: 3025, 1636, 1557, 1462, 1304, 1032, 747. HRMS: m/z calc. 453.1803 [M+H]⁺, obs. 453.1852 [M+H]⁺. Mp. 289.5-291.6° C. The purity of the compound was checked by HPLC (Rt=6.22 min) and was found to be 98.85% pure.

### 17-Cyclopropylmethyl-3,14β-dihydroxy-4,5α-epoxy-6α-(3′-thienylacetamido)morphinan hydrochloride (33)

Compound 33 was synthesized as shown in the general procedure with 44.64% yield. ¹H NMR (400 MHz, DMSO-d₆): δ. 9.22 (s, 1H, exchangeable), 8.81 (s, 1H, exchangeable), 7.93 (d, J=7.9 Hz, 1H, exchangeable), 7.46 (dd, J=5.0, 3.0 Hz, 1H), 7.28 (t, J=2.0 Hz, 1H), 7.06 (dd, J=4.9, 1.3 Hz, 1H), 6.73 (d, J=8.1 Hz, 1H), 6.57 (d, J=8.1 Hz, 1H), 6.19 (s, 1H, exchangeable), 4.62 (d, J=4.0 Hz, 1H), 4.39 (s, 1H), 3.87 (d, J=6.9 Hz, 1H), 3.51 (s, 2H), 3.06 (dd, J=19.7, 7.7 Hz, 2H), 2.95 (d, J=14.2 Hz, 1H), 2.72 (m, 1H), 2.42 (m, 1H), 1.83 (dd, J=15.9, 8.7 Hz, 1H), 1.62 (d, J=13.5 Hz, 1H), 1.42 (dd, J=15.3, 9.5 Hz, 2H), 1.02 (m, 2H), 0.69 (m, 1H), 0.61 (m, 1H), 0.47 (m, 1H), 0.40 (m, 1H). ¹³C NMR (100 MHz, DMSO-d₆) δ: 169.2, 145.9, 138.8, 136.1, 128.7, 128.6, 125.5, 122.0, 122.0, 119.0, 118.1, 87.3, 69.2, 61.0, 56.9, 45.1, 36.8, 30.1, 29.1, 23.4, 19.5, 5.6, 5.1, 2.5. IR (diamond, cm⁻¹) vₘₐₓ: 3133, 1629, 1538, 1457, 1320, 1034, 746. HRMS: m/z calc. 467.1960 [M+H]⁺, 489.1858 [M+Na]⁺, obs. 467.2010 [M+H]⁺, 489.1820 [M+Na]⁺. Mp. 196-198.2° C. The purity of the compound was checked by HPLC (Rt=6.47 min) and was found to be 97.35% pure.

### 17-Cyclopropylmethyl-3,14β-dihydroxy-4,5α-epoxy-6β-(3′-thienylacetamido)morphinan hydrochloride (34)

Compound 34 was synthesized as shown in the general procedure with 62.31% yield. ¹H NMR (400 MHz, DMSO-d₆): δ. 9.34 (s, 1H, exchangeable), 8.83 (s, 1H, exchangeable), 8.34 (d, J=7.9 Hz, 1H), 7.46 (dd, J=4.9, 2.9 Hz, 1H), 7.25 (dd, J=2.9, 1.3 Hz, 1H), 7.04 (dd, J=4.9, 1.3 Hz, 1H), 6.72 (d, J=8.1 Hz, 1H), 6.64 (d, J=8.2 Hz, 1H), 6.17 (s, 1H, exchangeable), 4.59 (d, J=7.8 Hz, 1H), 3.84 (d, J=5.6 Hz, 1H), 3.05 (m, 2H), 2.86 (ddt, J=11.0, 7.7, 3.5 Hz, 1H), 2.73 (dd, J=4.9, 1.4 Hz, 1H), 2.42 (m, 2H), 1.73 (m, 2H), 1.51 (dt, J=12.8, 4.4 Hz, 1H), 1.44 (d, J=9.8 Hz, 1H), 1.33 (td, J=13.8, 3.1 Hz, 1H), 1.07 (m, 1H), 0.68 (m, 1H), 0.60 (m, 1H), 0.51 (m, 1H), 0.41 (m, 1H). ¹³C NMR (100 MHz, DMSO-d₆) δ: 169.4, 142.0, 141.2, is 135.9, 129.5, 128.5, 125.6, 122.0, 120.5, 119.2, 117.9, 89.8, 69.6, 61.6, 56.6, 50.8, 46.4, 45.5, 37.2, 29.2, 27.3, 23.5, 22.9, 5.6, 5.0, 2.5. IR (diamond, cm⁻¹) vₘₐₓ: 3061, 1644, 1540, 1457, 1325, 1034, 745. HRMS: m/z calc. 467.1960 [M+H]⁺, obs. 467.2019 [M+H]⁺. Mp. 249.6-252.2° C. The purity of the compound was checked by HPLC (Rt=6.29 min) and was found to be 98.08% pure.

### 17-Cyclopropylmethyl-3,14β-dihydroxy-4,5α-epoxy-6α-[3′-(thiophen-3″-yl)propanamido]morphinan hydrochloride(35)

Compound 35 was synthesized as shown in the general procedure with 42.61% yield. ¹H NMR (400 MHz, DMSO-d₆): δ. 9.20 (s, 1H, exchangeable), 8.83 (s, 1H, exchangeable), 7.70 (d, J=8.0 Hz, 1H, exchangeable), 7.44 (dd, J=4.9, 2.9 Hz, 1H), 7.18 (d, J=2.9 Hz, 1H), 7.01 (dd, J=4.9, 1.3 Hz, 1H), 6.72 (d, J=8.1 Hz, 1H), 6.57 (d, J=8.1 Hz, 1H), 6.22 (s, 1H, exchangeable), 4.59 (d, J=4.1 Hz, 1H), 4.41 (ddd, J=13.1, 8.3, 4.0 Hz, 1H), 3.87 (d, J=6.9 Hz, 1H), 3.05 (dd, J=19.6, 7.0 Hz, 2H), 2.95 (m, 2H), 2.89-2.77 (m, 3H), 2.72 (m, 1H), 1.84 (dt, J=15.1, 9.3 Hz, 1H), 1.63 (m, 1H), 1.41 (dd, J=15.9, 10.4 Hz, 2H), 0.95 (m, 2H), 0.69 (m, 1H), 0.62 (m, 1H), 0.48 (m, 1H), 0.40 (m, 1H). ¹³C NMR (100 MHz, DMSO-d₆) δ: 170.9, 145.9, 141.5, 138.7, 128.7, 128.3, 125.7, 122.0, 120.3, 87.4, 69.2, 61.0, 56.9, 45.1, 44.84, 35.8, 31.8, 30.1, 29.1, 25.6, 23.42, 15.1, 5.6, 5.1, 2.5. IR (diamond, cm⁻¹) vₘₐₓ: 2945, 1640, 1538, 1455, 1319, 1032, 747. HRMS: m/z calc. 481.2116 [M+H]⁺, 503.2014 [M+Na]⁺, obs. 481.2154 [M+H]⁺, 503.1965 [M+Na]⁺. Mp. 175.4-178° C. The purity of the compound was checked by HPLC (Rt=6.85 min) and was found to be 96.47% pure.

### 17-Cyclopropylmethyl-3,14β-dihydroxy-4,5α-epoxy-6β-[3′-(thiophen-3″ yl)propanamido]morphinan hydrochloride (36)

Compound 36 was synthesized as shown in the general procedure with 78.26% yield. ¹H NMR (400 MHz, DMSO-d₆): δ. δ 9.33 (s, 1H, exchangeable), 8.80 (s, 1H, exchangeable), 8.13 (d, J=7.9 Hz, 1H, exchangeable), 7.44 (dd, J=4.9, 2.9 Hz, 1H), 7.16 (dd, J=2.9, 1.3 Hz, 1H), 7.00 (dd, J=4.9, 1.3 Hz, 1H), 6.72 (d, J=8.1 Hz, 1H), 6.65 (d, J=8.2 Hz, 1H), 6.11 (s, 1H, exchangeable), 4.54 (d, J=7.9 Hz, 1H), 3.82 (d, J=5.5 Hz, 1H), 3.08 (d, J=5.8 Hz, 1H), 3.02 (dd, J=11.7, 5.0 Hz, 2H), 2.82 (tt, J=7.3, 4.2 Hz, 3H), 2.74 (m, 1H), 2.39 (q, J=7.6 Hz, 3H), 1.68 (d, J=12.6 Hz, 2H), 1.47 (m, 2H), 1.34 (m, 1H), 1.22 (m, 1H), 1.1 (m, 1H), 0.68 (m, 1H), 0.60 (m, 1H), 0.50 (m, 1H), 0.41 (m, 1H). ¹³C NMR (100 MHz, DMSO-d₆) δ: 171.0, 142.1, 141.4, 141.2, 129.6, 128.3, 125.6, 120.5, 120.5, 119.2, 117.8, 89.8, 69.6, 61.7, 56.6, 50.5, 48.5, 45.5, 36.4, 29.3, 27.2, 25.5, 23.5, 22.9, 5.6, 5.0, 2.5. IR (diamond, cm⁻¹) vₘₐₓ: 3053, 1644,1530,1455,1298, 1087, 750. HRMS: m/z calc. 481.2116 [M+H]⁺, obs. 481.2174 [M+H]⁺. Mp. 209.8-211.6° C. The purity of the compound was checked by HPLC (Rt=6.66 min) and was found to be 99.50% pure.

### Biological Evaluation of Drugs.

Morphine (morphine sulfate pentahydrate salt) was purchased from Mallinckrodt (St. Louis, MO) or provided by the National Institute of Drug Abuse (NIDA). Naltrexone and naloxone were purchased as their hydrochloride salts from Sigma-Aldrich (St. Louis, MO). All drugs and test compounds were dissolved in pyrogen-free isotonic saline (Baxter Healthcare, Deerfield, IL) or sterile-filtered distilled/deionized water. All other reagents and radioligands were purchased from either Sigma-Aldrich or Thermo Fisher.

### Animals.

Male Swiss Webster mice (25-35 g, 6-8 weeks, Harlan Laboratories, Indianapolis, IN) were housed in a temperature-controlled (20-22° C.) AAALAC-accredited facility in which they had ad libitum access to food and water. The mice were maintained on a 12 h/12 h light-dark cycle (0600-1800 lights on) for the duration of the experiment and were tested during the light segment of this cycle. Mice arrived at the vivarium housed 4/cage and, following 1-week habituation, were separated into individual cages. Mice were allowed to acclimate to individual caging for at least 24 h and then were randomly assigned to the various treatment conditions before the start of studies. Experimenters were blinded to these treatment conditions during the duration of the experiment and data analysis. No adverse events occurred during the experiment, and no mice were excluded from data analysis. Protocols and procedures (Animal Welfare Assurance Number D16-00180) were approved by the Institutional Animal Care and Use Committee (IACUC) at the Virginia Commonwealth University Medical Center and complied with the recommendations of the IASP (International Association for the Study of Pain).

### In Vitro Competitive Radioligand Binding Assay.

The competition binding assay was conducted using the monoclonal mouse opioid receptors expressed in Chinese hamster ovary (CHO) cell lines (monoclonal human δ opioid receptor was used in the DOR assay). In this assay, 20-30 μg of membrane protein was incubated with the corresponding radioligand in the presence of different concentrations of test compounds in TME buffer (50 mM Tris, 3 mM MgCl₂, and 0.2 mM EGTA, pH 7.7) for 1.5 h at 30° C. The bound radioligand was separated by filtration using the Brandel harvester. Specific (i.e., opioid receptor-related) binding at the MOR, KOR, and DOR was determined as the difference in binding obtained in the absence and presence of 5 μM naltrexone, U50,488, and SNC80, respectively. All competition binding data were transformed to % Bound=specific binding in the presence of competing ligand/specific binding in the absence of competing ligand×100%.

### In Vitro [³⁵S]GTPγS Functional Assay.

The [³⁵S]GTPγS functional assay was conducted to determine the efficacy of the compounds at the MOR. In this assay, 10 μg of MOR-CHO membrane protein was incubated in a final vol of 500 μL containing TME with 100 mM NaCl, M GDP, 0.1 nM [³⁵S]GTPγS, and varying concentrations of the compound under investigation for 1.5 h in a 30° C. water bath. The Bradford protein assay was utilized to determine and adjust the concentration of protein required for the assay. Nonspecific binding was determined with 20 μM unlabeled GTPγS. Furthermore, 3 μM DAMGO was included in the assay as the maximally effective concentration of a full agonist for the MOR. After incubation, the bound radioactive ligand was separated from the free radioligand by filtration through a GF/B glass fiber filter paper and rinsed three times with ice-cold wash buffer (50 mM Tris-HCl, pH 7.2) using the Brandel harvester. Bound radioactivity was determined by liquid scintillation counting. All assays were determined in duplicate and repeated at least three times. Net stimulated [³⁵S]GTPγS binding was defined as agonist-stimulated minus basal binding in the absence of agonist. Percent of DAMGO-stimulated [³⁵S]GTPγS binding was defined as (net-stimulated binding by ligand/net-stimulated binding by 3 μM DAMGO)×100%.

### Data Analysis of Receptor Binding and [³⁵S]GTPγS Functional Assay.

The assays of all samples were conducted in duplicate and repeated at least three times for a total of >3 independent determinations. Results were reported as mean values ±SEM. Concentration-effect curves were fit by nonlinear regression to four parameter model with the minimum constrained to 0, using GraphPad Prism software, to determine Hill deficient, EC₅₀ and Eₘₐₓ values. IC₅₀ values were obtained from nonlinear regression fitting to four parameter model with the maximum (absence of competitor) constrained 100% and the minimum constrained to 0 using GraphPad Prism software. By using the Cheng-Prusoff equation Kᵢ=IC₅₀/[1+([L]/K_{D})], where [L] is the concentration of the competitor and K_{D} is the K_{D} of the radioligand, binding Kᵢ values were determined from IC₅₀ values.

### Warm-Water Tail Immersion Assay.

Antinociceptive effect of synthesized compounds was determined using the warm-water tail immersion assay. Swiss Webster mice (six male mice for each group, 25-35 g, 6-8 weeks old) were used in this assay. Antinociception for all compounds was examined in male Swiss Webster mice. The water bath temperature was set as 56±0.1° C. The baseline latency (control) was determined before administration of the compounds to the mice, and only mice with a baseline latency of 2 to 4 s were used. In the agonism study, the tail immersion was done 20 min (time that morphine effect starts to peak) after injecting the test compounds subcutaneously (s.c.). To prevent tissue damage, a 10 s maximum cutoff time was imposed. Antinociceptive response was calculated as the percentage of maximum possible effect (% MPE), where % MPE=[(test−control latency)/(10−control latency)]×100. When being studied for their antagonist effects to morphine, the test compounds were given (s.c.) 5 min before morphine. The tail immersion test was then conducted 20 min after giving morphine (s.c.). % MPE was calculated for each mouse. AD₅₀ values were calculated using the least-squares linear regression analysis followed by calculation of 95% confidence interval by the Bliss method.

### Opioid-Withdrawal Studies.

Swiss Webster mice (six male mice for each group, 25-35 g, 6-8 weeks old) were used for opioid-withdrawal studies. Following a previously reported protocol, a 75 mg morphine pellet was implanted into the back of the mice, and the mice were allowed to recover in their home cages. Before being tested, a 30 min period was allowed for habituation to an open-topped, square, clear Plexiglas observation chamber (26×26×26 cm³) with lines partitioning the bottom into quadrants. All drugs and test compounds were administered (s.c.). The withdrawal was precipitated 72 h from pellet implantation with naloxone (1 mg/kg, s.c.) or the test compounds at varying doses. Withdrawal commenced within 3 min after antagonist administration. Escape jumps, paw tremors, and wet dog shakes were quantified by counting their occurrences over 20 min for each mouse. The data are presented as the mean±SEM.

### Caco-2 Permeability Studies.

Human epithelial colorectal adenocarcinoma (Caco-2) cells (HTB-37) were cultured in T75 flasks using complete Dulbecco's Modified Eagles Medium (DMEM) containing 10% fetal bovine serum (FBS), 1% glutamine, 1% penicillin and 1% streptomycin, at 37° C. in a 5% CO₂ atmosphere. Cells were passaged at 80-90% confluency using 0.05% trypsin-EDTA and the medium was changed every other day. Following this, the cells were trypsinized, suspended in medium and applied to a Millipore 96-well plate where they were cultured as monolayers at a density of 25,000 cells/well. The cells were incubated in a 37° C./5% CO₂ incubator to allow cell attachment and proliferation. Media was changed every 2-3 days for 21 days when cells reached 100% confluency. For Apical→Basolateral (A→B) permeability, 10 μM compound 25 was added to the apical (A) side and the amount of permeation determined on the basolateral (B) side; for Basolateral→Apical (B→A) permeability, 10 μM compound 25 was added to the B-side and the amount of permeation was determined on the A side. The A-side buffer contained 100 μM lucifer yellow dye, in Transport Buffer (1.98 g/L glucose in 10 mM HEPES, lx Hank's Balanced Salt Solution) pH 7.4, and the B-side buffer used was the Transport Buffer at pH 7.4. Caco-2 cells were incubated with 10 μM compound 25 in these buffers for 1 h. Ranitidine and Colchicine (low permeability), Labetalol and Propranolol (high permeability) were used as controls. At the end of the assay, donor and receiver side solution samples were collected, quenched by 100% methanol containing an internal standard and centrifuged at 5000 rpm for 10 min at 4° C. Following centrifugation, the supernatant for donor and receiver side samples was analyzed by HPLC-MS/MS to determine peak area ratios.

Data was expressed as Papp (cm/s):

$${P}_{app} = \frac{( VR * Cr , end )}{dt} * \frac{1}{A * ( CD , mid - Cr , mid )} \qquad Equation 1$$

where VR is the volume of the receiver chamber. CR, end is the concentration of the test compound in the receiver chamber at the end time point, dt is the incubation time and A is the surface area of the cell monolayer. CD, mid is the calculated mid-point concentration of the test compound in the donor side, which is the mean value of the donor concentration at time 0 minute and the donor concentration at the end time point. CR, mid is the mid-point concentration of the test compound in the receiver side, which is one half of the receiver concentration at the end time point. Concentrations of the test compound were expressed as peak areas of the test compound.

### In Vivo BBB Penetration Studies.

Swiss Webster mice (3 mice each time point, were given compound 25 (10 mg/kg, s.c.) or vehicle. At 5-, 10- and 30-min time points post administration, the mice were decapitated, and brain samples and blood samples were collected. Blood samples were centrifuged for 10 min at 15000 g at 4° C. following which plasma was collected. Brain and plasma samples were stored at −80° C. until further analysis.

### LCMS/MS Analysis.

The identification and quantification of compound 25 in mouse plasma and brain was performed using a modification of a previously described method with naloxone-d₅ as the internal standard. Chromatographic separation of compound 25 and naltrexone-d₅ was achieved using a Shimadzu Nexera X2 liquid chromatography system with a Zorbax XDB-C18 4.6×75 mm, 3.5-micron column (Agilent Technologies, Santa Clara, CA). Mobile phase A consisted of water with 1 g/L ammonium formate and 0.1% formic acid and mobile Phase B consisted of methanol. The flow rate was 1 mL/min. The systems detector was a Sciex 6500 QTRAP system with an IonDrive Turbo V source for TurbolonSpray® (Sciex, Ontario, Canada). The following quantification and qualifying transition ions were monitored in positive multiple reaction monitoring mode with collisions energies in parentheses: compound 25 453 >435 (27), 453>308 (35) and 455>267(43); naloxone-d₅ 333>212 (45), 333>315 (25) and 333 >273. Concentrations were determined by linear regression plot based on peak area ratios of the calibrators.

### Statistical Analysis.

One-way ANOVA followed by the post-hoc Dunnett test were performed to assess the significance using GraphPad Prism software (GraphPad Software, San Diego, CA).

### Example 2. Rational Design, Chemical Syntheses, and Biological Evaluations of Peripherally Selective Mu Opioid Receptor Ligands as Potential Opioid Induced Constipation Treatment

### Structure-Based and Physicochemical Property-Driven Drug Design.

The compound NAP possesses high MOR selectivity over the KOR and DOR. As NAP was observed to have therapeutic effects both systematically and peripherally, a series of SAR studies have been carried out to dissociate its peripheral nervous system (PNS)-favored characteristics from the central nervous system (CNS)-favored, and vice versa, in order to develop novel treatments for opioid use disorders and OIC. An aromatic moiety in the side chain was proposed to be maintained in the lead optimization for CNS- or PNS-targeted MOR antagonists.

To further limit MOR antagonism by these ligands to the periphery, the focus of studies disclosed here has been on the design and syntheses of MOR antagonists impermeable through the BBB, which is a primary and critical physical barrier between the CNS and periphery. One of the approaches established for assessing the potential of small molecules to penetrate the BBB is the in silico prediction and calculation based on physicochemical properties. To define the physicochemical properties space for CNS drug design, scientists at Pfizer developed a weighted scoring approach, called “CNS MPO (multiparameter optimization)” algorithm. In this scoring algorithm, six fundamental physicochemical properties, i.e., C log P (calculated partition coefficient), C log D (calculated distribution coefficient at pH 7.4), TPSA (topological polar surface area), MW (molecular weight), HBD (number of hydrogen-bond donors), and pKa (dissociation constant) are included (Wager, et al. ACS Chem. Neurosci. 2016, 7 (6), 767-775). Each property is weighted equally and defined as TO with values between 0 and 1. Therefore, the collective CNS MPO score of a chemical entity may range from 0 to 6.0, with a desirable score greater than 4.0 as a widely used cut-off to select hits in CNS drug discovery programs. Meanwhile, it should be noted that MPO score greater than 4 as a cut-off is solely based on the observation that 74% of already-marketed CNS-targeting drugs demonstrated a high CNS MPO (>4) while a reasonable number of exceptions probably still exist. Applying CNS MPO seems a practical approach for balancing multiple variables without the penalty of hard cut-offs and can be used prospectively in molecular design. In this context, we hypothesized that a CNS MPO score lower than 4.0 would suggest PNS-acting potential of designed small molecules.

To validate our hypothesis, the CNS MPO scores were first calculated for NAP, MNTX, naldemedine, and naloxegol by adopting the CNS MPO desirability tool (Table 8). Both naldemedine and naloxegol showed CNS MPO scores lower than 4.0, which is in line with their PNS dominated properties. Meanwhile, NAP and MNTX showed CNS MPO scores of 4.38 and 4.64, respectively, suggesting their potential CNS-targeting characteristics, which is in agreement with the fact that both of them carried centrally-mediated effects that conferred peripheral selectivity.

Combined with the isosterism concept, we introduced a highly polar moiety, i.e., a pyrazolyl or imidazolyl ring, to replace the pyridyl ring in NAP in order to increase TPSA and HBD as well as to decrease C log P and C log D of the whole molecule, in the hope to further lower CNS MPO scores. Moreover, as heteroaromatic ring systems, pyrazolyl and imidazolyl are also expected to maintain the essential interactions with Trp318^{7.35} and Lys303^{6.58}. In the newly designed agents (1-24, Scheme 2), we also altered the stereochemistry at C(6) (α or β), the distance between the aromatic ring and the morphinan skeleton, and the linker substitution position on the aromatic rings to further explore the preferred physiochemical features of NAP analogs as PNS agents. Compounds 1-24 were calculated to possess CNS MPO scores ranging from 3.56 to 3.76, all lower than 4.0. It should be noted that all designed compounds have increased TPSA and HBD as well as decreased C log P and C log D values as expected (Table 8). In summary, we generated a focused library enriched with possibly active and selective molecules for the MOR, which could be more efficient to discover new lead compounds and drug candidates with PNS-selective potency.

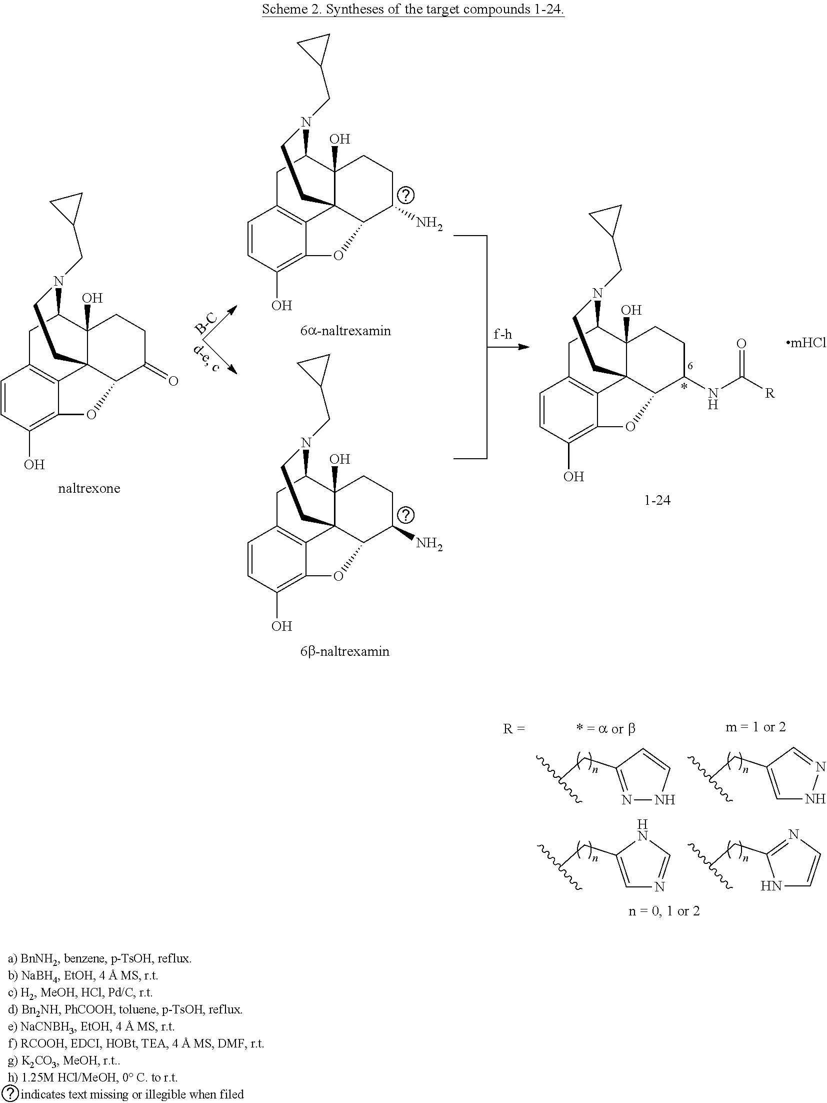

### Chemistry

The chemical syntheses of target compounds 1-24 were performed following the synthetic route outlined in Scheme 1. Briefly, 6α- or 6β-naltrexamine was prepared by stereoselective reduction amination of naltrexone with benzylamine or dibenzylamine, respectively, followed by debenzylation under catalytic hydrogenation condition (Yuan, et al. ACS Chem. Neurosci. 2011, 2 (7), 346-351). A variety of commercially available pyrazole- or imidazole-bearing carboxylic acids, were coupled with 6α- or 6β-naltrexamine employing the EDCI/HOBt method. After treatment with potassium carbonate in methanol, the 6-position monosubstituted free bases were furnished. These free bases were then converted to their hydrochloric acid salt forms, fully characterized and submitted for in vitro and in vivo pharmacological studies.

### In Vitro Radioligand Binding and MOR [31S]-GTPγS Functional Assays.

To characterize the binding affinity and selectivity profiles of all newly synthesized compounds on the three opioid receptors, in vitro competitive radioligand binding assays were performed as previously described (Zheng et al. J. Med. Chem. 2019, 62 (2), 561-574). The results are summarized in Table 9 and 10.

TABLE 9
Binding affinity, selectivity, and MOR [³⁵S]-GTPγS functional assay results of Compounds 1-12 (6α-configuration)^{a}

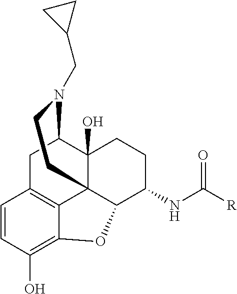

| | | Ki (nM) | | | Selectivity | | MOR [³⁵S]-GTPγS Binding | |
|---|---|---|---|---|---|---|---|---|
| Compd | R | μ | δ | κ | δ/μ | κ/μ | EC₅₀ (nM) | % Eₘₐₓ of DAMGO |
| MNTX^{b} | | 5.50 ± 1.11 | 3454 ± 305 | 32.1 ± 1.44 | 628 | 6 | >10000 | NA^{c} |
| NAP^{d} | 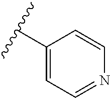 [SMILES: *c1ccncc1] | 0.37 ± 0.07 | 278 ± 8.0 | 60.7 ± 5.6 | 747 | 163 | 1.14 ± 0.38 | 22.7 ± 0.84 |
| 1 | 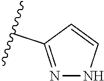 [SMILES: *c1cc[nH]n1] | 0.61 ± 0.05 | 21.2 ± 6.08 | 1.05 ± 0.28 | 35 | 2 | 2.33 ± 0.89 | 19.6 ± 1.08 |
| 2 | 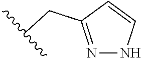 [SMILES: *Cc1cc[nH]n1] | 1.05 ± 0.06 | 195 ± 15.1 | 154 ± 28.7 | 186 | 147 | 6.17 ± 0.94 | 21.5 ± 0.63 |
| 3 | 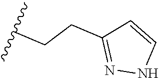 [SMILES: *CCc1cc[nH]n1] | 0.50 ± 0.04 | 217 ± 16.2 | 2.75 ± 0.74 | 433 | 6 | 4.22 ± 0.51 | 22.2 ± 0.40 |
| 4 | 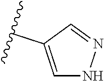 [SMILES: *c1cn[nH]c1] | 0.59 ± 0.02 | 37.5 ± 1.34 | 4.70 ± 1.12 | 64 | 8 | 1.24 ± 0.11 | 23.1 ± 2.74 |
| 5 | 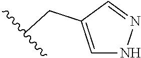 [SMILES: *Cc1cn[nH]c1] | 0.88 ± 0.26 | 3445 ± 362 | 104 ± 22.4 | 3922 | 118 | 8.17 ± 1.02 | 28.4 ± 1.40 |
| 6 | 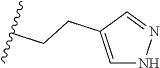 [SMILES: *CCc1cn[nH]c1] | 1.09 ± 0.14 | 91.2 ± 6.93 | 39.5 ± 8.98 | 83 | 36 | 12.3 ± 1.53 | 14.2 ± 2.26 |
| 7 | 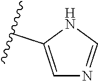 [SMILES: *c1cnc[nH]1] | 1.11 ± 0.04 | 32.5 ± 6.70 | 2.76 ± 0.44 | 29 | 2 | 4.42 ± 1.66 | 20.7 ± 1.57 |
| 8 | 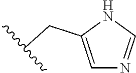 [SMILES: *Cc1cnc[nH]1] | 1.34 ± 0.11 | 79.4 ± 0.83 | 3.57 ± 0.56 | 59 | 3 | 3.61 ± 0.87 | 21.0 ± 1.62 |
| 9 | 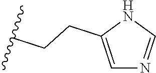 [SMILES: *CCc1cnc[nH]1] | 0.20 ± 0.01 | 21.5 ± 2.96 | 1.21 ± 0.29 | 106 | 6 | 9.47 ± 1.32 | 25.4 ± 2.65 |
| 10 | 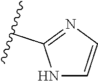 [SMILES: *c1ncc[nH]1] | 0.47 ± 0.01 | 70.9 ± 14.7 | 1.77 ± 0.36 | 152 | 4 | 2.01 ± 1.72 | 7.85 ± 1.02 |
| 11 | 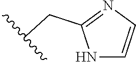 [SMILES: *Cc1ncc[nH]1] | 0.83 ± 0.01 | 17.7 ± 2.80 | 3.45 ± 0.36 | 21 | 4 | 1.81 ± 0.10 | 19.2 ± 1.54 |
| 12 | 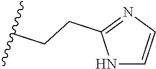 [SMILES: *CCc1ncc[nH]1] | 0.69 ± 0.03 | 3.94 ± 1.28 | 0.79 ± 0.18 | 6 | 1 | 4.26 ± 1.20 | 21.7 ± 0.47 |

^{a}The values are the mean ± SEM of at least three independent experiments.
^{b}Data have been reported in Kanemasa et al. Neurogastroenterol. Motil. Off. J. Eur.
Gastrointest. Motil. Soc. 2019, 31 (5), e13563_ENRE_42, and are presented here for comparison purposes.
Human recombinant opioid receptors were used in the assay.
_{c}NA: not applicable.
^{d}Data have been reported in Li et al. J. Med. Chem. 2009, 52 (5), 1416-1427_ENREF_42, and are presented here for comparison purposes.

As shown in Table 9, all 6α-compounds 1-12 retained high binding affinity, subnanomolar to one-digit nanomolar, for the MOR. All compounds exhibited higher binding affinities than that of MNTX (K_{i, MOR}=5.50+1.11 nM), and most compounds possessed comparable Kᵢ values to NAP (K_{i, MOR}=0.37 i 0.07 nM). It was also observed that compounds with a carboxamido linker showed higher MOR binding affinity than compounds with an acetamido linker (1 vs 2, 4 vs 5, 7 vs 8, 10 vs 11), which demonstrated the same trend as their corresponding CNS MPO scores (3.76 vs 3.66), while they may not show higher affinity than the compounds with an n-propanamido linker. In addition, nine out of twelve (1, 3, 4, 7-12) also exhibited one-digit nanomolar binding affinity for either the KOR or DOR, but these compounds still preserved reasonable δ/μ and κ/μ selectivity. More particularly, compounds 2 and 5 demonstrated at least a hundred-fold selectivity for the MOR over both the KOR and DOR. Also, in general, compounds bearing a pyrazolyl ring (1-6) presented higher selectivity towards the MOR than the ones bearing an imidazolyl ring (7-12).

Among the 6β-configuration compounds (Table 10), it was observed that all 6β-compounds also maintained high binding affinity for the MOR. Moreover, all 6β-compounds possessed subnanomolar Kᵢ values and showed higher binding affinity for the MOR than MNTX and their 6α-counterparts except for 21. Meanwhile, among the six compounds (13-18) bearing a pyrazolyl ring, the ones with an acetamido linker showed the highest MOR affinity (13 vs 14 vs 15, 16 vs 17 vs 18), while for the compounds bearing an imidazolyl ring, the ones with a carboxamido linker did the same (19 vs 20 vs 21, 22 vs 23 vs 24). Unlike 6α-compounds, the binding affinities of 13-24, for the DOR were all at most double-digit nanomolar, thereby increasing the δ/μ selectivity. Particularly, compounds 14, 15, 17-19, 22, and 23 presented hundreds-fold δ/μ selectivity. The x/selectivity was also preserved for 13-23. Though the newly-designed compounds showed lower selectivity for the MOR than NAP, most compounds were still MOR-selective and some compounds (14, 17-19) continued to exhibit high selectivity over both the KOR and DOR.

TABLE 10
Binding affinity, selectivity, and MOR [³⁵S]-GTPγS functional assay results of Compounds 13-24 (6β-configuration)^{a}

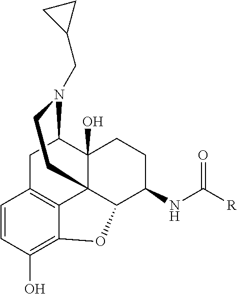

| | | Ki (nM) | | | Selectivity | | MOR [³⁵S]-GTPγYS Binding | |
|---|---|---|---|---|---|---|---|---|
| Compd | R | μ | δ | κ | δ/μ | κ/μ | EC₅₀ (nM) | % Emax of DAMGO |
| MNTX^{b} | | 5.50 ± 1.11 | 3454± 305 | 32.1 ± 1.44 | 628 | 6 | >10000 | NA^{c} |
| NAP^{d} | 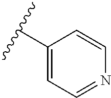 [SMILES: *c1ccncc1] | 0.37 ± 0.07 | 278 ± 8.0 | 60.7 ± 5.6 | 747 | 163 | 1.14 ± 0.38 | 22.7 ± 0.84 |
| 13 | 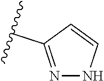 [SMILES: *c1cc[nH]n1] | 0.28 ± 0.06 | 14.5 ± 3.77 | 0.95 ± 0.15 | 52 | 3 | 1.83 ± 0.59 | 10.9 ± 0.90 |
| 14 | 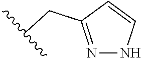 [SMILES: *Cc1cc[nH]n1] | 0.20 ± 0.02 | 36.8 ± 5.50 | 5.05 ± 0.82 | 186 | 26 | 2.50 ± 0.56 | 20.4 ± 2.06 |
| 15 | 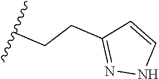 [SMILES: *CCc1cc[nH]n1] | 0.21 ± 0.03 | 42.2 ± 5.87 | 2.00 ± 0.22 | 202 | 10 | 2.76 ± 0.54 | 13.2 ± 1.96 |
| 16 | 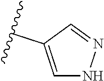 [SMILES: *c1cn[nH]c1] | 0.65 ± 0.11 | 46.8 ± 12.7 | 2.51 ± 0.20 | 72 | 4 | 1.77 ± 0.24 | 16.3 ± 0.85 |
| 17 | 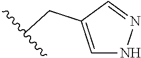 [SMILES: *Cc1cn[nH]c1] | 0.17 ± 0.02 | 66.6 ± 10.3 | 13.9 ± 3.57 | 390 | 82 | 1.52 ± 0.12 | 22.2 ± 1.86 |
| 18 | 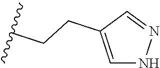 [SMILES: *CCc1cn[nH]c1] | 0.30 ± 0.03 | 97.8 ± 14.0 | 12.3 ± 1.56 | 322 | 40 | 4.28 ± 1.00 | 10.7 ± 1.19 |
| 19 | 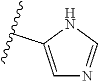 [SMILES: *c1cnc[nH]1] | 0.58 ± 0.02 | 209 ± 21.6 | 18.8 ± 2.36 | 362 | 32 | 1.15 ± 0.73 | 10.0 ± 1.81 |
| 20 | 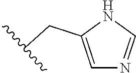 [SMILES: *Cc1cnc[nH]1] | 0.82 ± 0.10 | 74.5 ± 1.18 | 4.82 ± 0.55 | 91 | 6 | 4.64 ± 0.44 | 20.9 ± 1.15 |
| 21 | 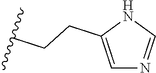 [SMILES: *CCc1cnc[nH]1] | 0.60 ± 0.10 | 35.5 ± 8.25 | 1.80 ± 0.28 | 60 | 3 | 3.19 ± 0.29 | 15.7 ± 1.05 |
| 22 | 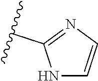 [SMILES: *c1ncc[nH]1] | 0.44 ± 0.04 | 146 ± 27.6 | 4.80 ± 0.56 | 335 | 11 | 1.74 ± 0.60 | 7.50 ± 1.37 |
| 23 | 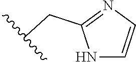 [SMILES: *Cc1ncc[nH]1] | 0.48 ± 0.02 | 89.8 ± 17.4 | 5.17 ± 0.61 | 188 | 11 | 2.07 ± 0.70 | 23.5 ± 1.29 |
| 24 | 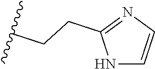 [SMILES: *CCc1ncc[nH]1] | 0.57 ± 0.03 | 55.8 ± 6.9 | 0.57 ± 0.09 | 99 | 1 | 1.24 ± 0.26 | 17.7 ± 0.99 |

^{a}The values are the mean ± SEM of at least three independent experiments.
^{b}Data have been reported in Li et al, supra and Kanemasa et al., supra_ENREF_42, and are presented here for comparison purposes.
Human recombinant opioid receptors were used in the assay.
^{c}NA: not applicable.
^{d}Data have been reported in Li et al, supra_ENREE_42, and are presented here for comparisonpurposes.

The [³⁵S]-GTPγS binding assay was carried out as well to determine the functionality on the MOR of each compound. As presented in Tables 9 and 10, all compounds showed low efficacy with % Eₘₐₓ values ranging from 7.50±1.37 to 23.5±1.29, which indicated these compounds may behave as MOR antagonists similarly to NAP (% Eₘₐₓ=22.7+0.84). The EC₅₀ value of MNTX was >10000 nM for MOR, indicating an insignificant agonist activity. Interestingly, the 6β-analogues, which shared the same C6 configuration with NAP, demonstrated lower efficacies than their 6α-counterparts (except for 23).

Taken together, the replacement of the pyridyl ring in NAP with its isosteric pyrazolyl and imidazolyl rings maintained high binding affinity, selectivity, and low-efficacy functionality at the MOR.

### In Vivo Warm-Water Tail Immersion Assay.

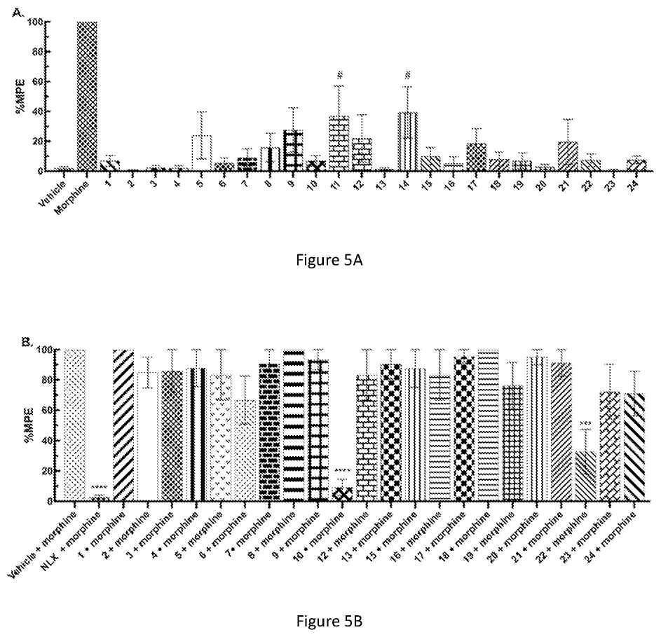

Warm-water tail immersion assay is a well-established assay for evaluating opioid analgesics, especially MOR agonists. The antinociceptive effects in tail withdrawal test have been found to involve both spinal and supraspinal level of the CNS and MOR antagonists that can effectively block the antinociception produced by MOR agonists are very possible central-acting agents. Hence, such practice has been employed to distinguish and preclude CNS-acting MOR ligands, either agonists or antagonists. More specifically, warm-water tail immersion pain model, which measures changes in latency of response to thermal stimulation at varying doses of tested compounds, has been widely used in our practice and others to test the in vivo acute agonistic or antagonistic effects of opioids and non-opioids. In this context, all 24 compounds were first evaluated for their acute antinociception tail-withdrawal assay (FIG. 5A). Two compounds, 11 and 14, showed moderate antinociceptive effects with 36.9±20.2% and 39.3±17.2% maximum possible effects (MPE) indicating their potential CNS activity. Subsequently, the other 22 compounds exhibiting no apparent CNS antinociception were tested for blockade of morphine's antinociception effect. As shown in FIG. 5B, only two compounds, 10 and 22, significantly antagonized morphine's antinociception effects to 9.2±5.4% MPE and 32.7±14.6% MPE, respectively. In the follow-up dose-response study (not shown), compound 10 was observed to possess an AD₅₀ of 5.3 mg/kg. The rest of the 20 compounds (1-9, 12-13, 15-21, 23-24) showed no evident antagonism against morphine in the tail immersion studies.

### Preliminary GI Tract Motility Study.

The carmine red dye study, charcoal meal test, and colonic bead expulsion assay (in vivo or ex vivo) are three commonly applied protocols to evaluate GI tract motility. The charcoal meal test and colonic bead expulsion measures small and large intestinal transit, respectively, while the carmine red dye method measures the whole GI tract mobility. The carmine red dye assay, thus, was employed as our preliminary OIC animal model to examine the in vivo effects of potential peripherally restricted MOR ligands.

All of the compounds 1-9, 12-13, 15-21, 23-24 showed very similar MOR binding affinity (Table 9 and 10), so based on their relatively higher MOR selectivity over the DOR and KOR and low MOR efficacy, compounds 2 and 5 (6α-configuration) and 17, 18 and 19 (6β-configuration) from two sub-series were first considered to be evaluated in the red dye studies. Meanwhile, since compounds 2, 5, 17, and 18 all contain a pyrazolyl ring while 19 contains an imidazolyl ring, and compound 17 possessed higher MOR selectivity than 18, therefore, compounds 2, 5, 17, and 19, two from each sub-series with structural diversity, were finally chosen to undergo the test first.

The _ENREF_1 carmine red dye study recorded the time required to defecate a red fecal pellet after oral administration. As shown in FIG. 6A, compared to the naïve group, 10 mg/kg morphine elongated the pellet defecation time by 124 minutes. Then the selected four compounds were administered subcutaneously (s.c.) to the mice to alleviate the constipation.

However, none of the compounds, though potent MOR antagonists/low efficacy partial agonists in vitro, were able to reduce the lengthened GI tract transit time (FIG. 6A). It was speculated that, because of the high TPSA, their passive permeability may be too low to allow them to distribute from the injection site to the GI tract. Therefore, the administration route was changed from systematically s.c. to oral gavage for shortening the distribution path and increasing the compound concentration at the action site. To exclude the water interference, vehicle was also given orally in the naïve group and morphine group (FIG. 6B). As presented in FIG. 6B, all four compounds, 2, 5, 17, and 19, successfully reversed morphine-induced constipation via oral administration by 50%, 39%, 40%, and 38%, respectively. No statistical significance was observed from the results, which might be partially due to their possessing similar CNS MPO scores. The oral administration route appeared to be feasible and favorable for these new compounds to elicit in vivo GI effects. Furthermore, these collective results, ineffectiveness via s.c. and effectiveness via p.o., not only indicated their applicability as potentially orally available agents, but also further demonstrated their PNS selectivity.

### KOR and DOR [³⁵S]-GTPγS Functional Assays

As noted, KOR activation may cause some adverse effects including sedation and dysphoria and DOR agonism may induce convulsion and also cause constipation. _ENREF_53 Due to the fact that compound 2 showed the best anti-constipation effect, and compound 19 exhibited almost the same efficacy in reversing morphine-induced constipation as those of 5 and 17 (FIG. 6B) while lower MOR efficacy, 2 (% Eₘₐₓ=21.5±0.63) and 19 (% Eₘₐₓ=10.0±1.81) were further characterized for their functionality on KOR and DOR in the [³⁵S]GTPγS binding assays, respectively. The results are shown in Table 11. MNTX was reported to possess EC₅₀ values greater than 10000 nM for both KOR and DOR, suggesting that it did not behave as a potent agonist for these two receptors. Compared to NAP, compound 2 exhibited similar moderate efficacy at the KOR but with a much lower potency, and relatively high efficacy at the DOR with a comparable potency. Meanwhile, compound 19 demonstrated much lower efficacy at the KOR than NAP and presented reasonably low efficacy at the DOR. Compared to compound 2 that could act as a KOR and DOR dual partial agonist, compound 19 might act as a KOR antagonist and DOR partial agonist with lower efficacy. Therefore, 19 might display fewer adverse effects, and was therefore selected for further carmine red dye studies.

TABLE 11
KOR and DOR [³⁵S]-GTPγS Functional Assay Results of Compounds 2 and 19.^{a}

| | KOR [³⁵S]-GTPγS binding | | DOR [³⁵S]-GTPγS binding | |
|---|---|---|---|---|
| Compounds | EC₅₀ (nM) | % Eₘₐₓ of U50, 488H | EC₅₀ (nM) | % Eₘₐₓ of SNC80 |
| 2 | 99.0 ± 18.3 | 49.9 ± 1.22 | 20.5 ± 4.02 | 69.3 ± 4.78 |
| 19 | 11.7 ± 2.01 | 9.03 ± 0.85 | 19.1 ± 5.91 | 22.1 ± 0.70 |
| NAP^{b} | 28.8 ± 14.4 | 45.5 ± 4.4 | 15.2 ± 15.2 | 10.2 ± 3.1 |
| MNTX^{c} | >10000 | NA^{d} | >10000 | NA |

^{a}All values are the mean ± SEM of at least three independent experiments.
^{b}Data have been reported in Yuan et al. supra_ENREF_39, and are presented here for comparison purposes.
^{c}Data have been reported in Li et al, supra and Kanemasa et al., supra_ENREF_42_ENREF_42, and are presented here for comparison purposes. Human recombinant opioid receptors were used in the assay.
^{d}NA: not applicable.

### Further Carmine Red Dye Study.

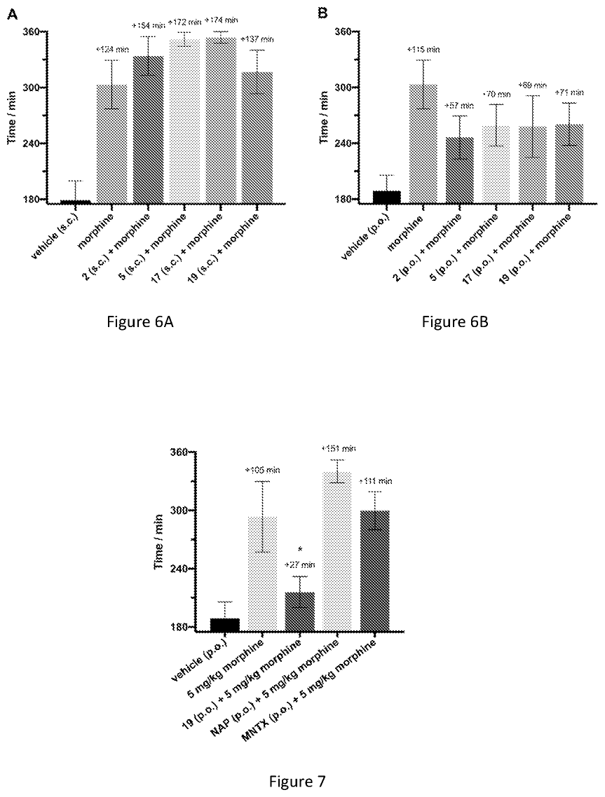

Characterized as an orally-active MOR-selective antagonist with no obvious CNS activity, compound 19 was then further examined in the OIC model to compare to NAP and MNTX. MNTX, when injected intraperitoneally, was reported to be effective to block the anti-transit effects of morphine (5 mg/kg, s.c.). In our studies MNTX was instead evaluated by oral gavage, a more clinically relevant administration route. At the same time, all compounds were also evaluated against the constipation caused by 5 mg/kg morphine. First, 5 mg/kg morphine was injected to validate the significant induction of OIC under our testing conditions. Then compound 19, NAP, and MNTX were administrated by oral gavage. As expected, compound 19 dramatically reversed the inhibitory effects of morphine on GI motility by 75% with reasonable significance (FIG. 7). Interestingly, no blockage was observed for NAP and MNTX, when given orally, of the GI motility inhibition by morphine. It is worth mentioning that we observed softened stool and increased pellets of poops in the MNTX group compared to the morphine group, though neither was quantitative. Another observation is that all the tested mice in the MNTX group possessed much less time to defecate the red fecal pellet than two tested mice in the morphine group though the average time of the MNTX group was similar to that of the morphine group. Both observations indicated that MNTX might exhibit somehow insignificant efficacy at a dose of 10 mg/kg in mice, while individual difference of mice may affect the test results. Although NAP was shown with high potency in alleviating OIC in previous studies, we postulated that maybe metabolites of NAP played major roles in its OIC reversal activity since the administration route was subcutaneous. However, it might not be the same case with MNTX because the active form of MNTX was MNTX itself and no metabolites were observed. The absolute oral bioavailability of MNTX in human subjects has not been determined while its bioavailability in male rats was very low (<1%) after oral administration. Indeed, there have been two studies reported to improve the oral bioavailability of MNTX in rats. We think that the very low oral bioavailability of MNTX in rodents, resulting in insufficient concentration of pharmacologically active molecules at the action sites, could be a very possible reason that explains its insignificant in vivo efficacy in the present study.

### Conclusions

By employing a structure-based and physicochemical property-driven drug design strategy, we designed and synthesized a set of NAP derivatives containing pyrazolyl and imidazolyl rings with decreased CNS MPO scores to improve PNS-selectivity. All the newly synthesized compounds maintained high binding affinity for the MOR and reasonable selectivity over the KOR and DOR. In the in vivo studies, most compounds showed marginal CNS effects. Among them, four selected compounds demonstrated efficaciousness in reversing OIC caused by morphine via oral administration but not subcutaneously. Taken together, these newly designed compounds seemed to possess reasonable PNS selectivity as designed. Most importantly, MPO score calculation worked well in our present practice, helping identify PNS-targeting lead compounds in an efficient way (with increased probability of success). As we can see from the in vivo studies, compound 19 was demonstrated to have dramatic improvement over NAP and MNTX in the carmine red dye assay to reverse OIC induced by morphine via p.o. route of administration. As the oral administration route is always clinically preferred, compared to MNTX, orally-effective compound 19 may serve as a promising new lead for further development as an OIC treatment option.

### Experimental Section

### Chemistry

All nonaqueous reactions were carried out under a pre-dried nitrogen gas atmosphere. All solvents and reagents were purchased from either Comi-blocks, Sigma-Aldrich, or Enamine LLC, and were used as received without further purification. Melting points (mp) were measured on an MPA100 OptiMelt automated melting point apparatus without correction. Analytical thin-layer chromatography (TLC) analyses were carried out on Analtech Uniplate F254 plates and flash column chromatography (FCC) was performed using silica gel (230-400 mesh, Merck). ¹H (400 MHz) and ¹³C (100 MHz) nuclear magnetic resonance (NMR) spectra were recorded on a Bruker Ultrashield 400 Plus spectrometer. Chemical shifts were expressed in δ units (ppm), using TMS as an internal standard, and J values were reported in hertz (Hz). Mass spectra were obtained on an Applied BioSystems 3200 Q trap with a turbo V source for Turbolon Spray. Analytical reversed-phase high performance liquid chromatography (HPLC) was performed on a Varian ProStar 210 system using Agilent Microsorb-MV 100-5 C18 column (250×4.6 mm). All analyses were conducted at an ambient temperature with a flow rate of 0.5 mL/min. HPLC eluent condition: acetonitrile/water (with 0.1% trifluoroacetic acid), acetonitrile increased from 40% to 100% in gradient within 20 min of test. The UV detector was set up at 210 nm. The injection volume was 5 μL. The purities of final compounds were calculated as the percentage peak area of the analyzed compound, and retention time (Rt) was presented in minutes. The purity of all newly synthesized compounds was identified as ≥95%.

### 17-Cyclopropylmethyl-3,14/l-dihydroxy-4,5α-epoxy-6α-(3′-pyrazolylcarboxamido)morphinan (1)

¹H NMR (400 MHz, DMSO-d₆) δ 9.36 (brs, 1H, exchangeable), 8.84 (brs, 1H, exchangeable), 7.82 (d, J=1.4 Hz, 1H), 7.55 (d, J=8.1 Hz, 1H, exchangeable), 6.73 (d, J=8.1 Hz, 1H), 6.69 (d, J=1.6 Hz, 1H), 6.59 (d, J=8.1 Hz, 1H), 6.29 (brs, 1H, exchangeable), 4.73 (d, J=3.8 Hz, 1H), 4.63-4.56 (m, 1H), 3.90 (d, J=6.7 Hz, 1H), 3.27-3.23 (m, 1H), 3.11-3.04 (m, 3H), 2.97-2.92 (m, 1H), 2.78-2.66 (m, 1H), 2.47-2.45 (m, 1H), 1.94-1.86 (m, 1H), 1.66 (dd, J=13.2, 2.6 Hz, 1H), 1.58-1.51 (m, 1H), 1.48-1.42 (m, 1H), 1.09-1.03 (m, 1H), 1.01-0.92 (m, 1H), 0.73-0.66 (m, 1H), 0.65-0.58 (m, 1H), 0.51-0.45 (m, 1H), 0.43-0.37 (m, 1H). ¹³C NMR (100 MHz, DMSO-d₆) δ 158.30, 143.13, 142.57, 136.22, 128.57, 126.24, 119.60, 116.98, 115.67, 102.73, 85.19, 66.70, 58.35, 54.47, 45.97, 42.72, 42.58, 42.15, 27.60, 26.66, 20.93, 17.42, 3.13, 2.71. HRMS calculated for C₂₄H₂₈N₄O₄ m/z: 436.2111. Found [M+H]⁺ (m/z): 437.2180. mp 269.7-271.7° C. dec. % Purity: 97.78. Rt: 6.709 min.

### 17-Cyclopropylmethyl-3,14β-dihydroxy-4,5α-epoxy-6α-(3′-pyrazolylacetamido)morphinan (2)

¹H NMR (400 MHz, DMSO-d₆) δ 8.87 (brs, 1H, exchangeable), 8.04 (d, J=7.9 Hz, 1H, exchangeable), 7.75 (m, 1H), 6.73 (d, J=8.1 Hz, 1H), 6.56 (d, J=8.1 Hz, 1H), 6.30 (m, 1H), is 4.59 (d, J=3.9 Hz, 1H), 4.44-4.37 (m, 1H), 3.92 (d, J=6.8 Hz, 1H), 3.62 (s, 2H), 3.31-3.22 (m, 2H), 3.08-2.93 (m, 3H), 2.75-2.65 (m, 1H), 2.49-2.41 (m, 2H), 1.92-1.84 (m, 1H), 1.62-1.58 (m, 1H), 1.43-1.37 (m, 2H), 0.99-0.92 (m, 1H), 0.70-0.65 (m, 1H), 0.63-0.57 (m, 1H), 0.51-0.45 (m, 1H), 0.42-0.36 (m, 1H). ¹³C NMR (100 MHz, DMSO-d₆) δ 167.87, 146.00, 142.90, 138.85, 132.77, 128.73, 122.08, 119.09, 118.32, 104.88, 87.40, 69.32, 60.92, 56.97, 45.15, 33.66, 30.11, 29.16, 23.47, 19.66, 5.67, 5.14, 2.54. HRMS calculated for C₂₅H₃₀N₄O₄ m/z: 450.2267. Found [M+H]⁺ (m/z): 451.2355, [M+Na]⁺ (m/z): 473.2178. mp 253.7-254.5° C. dec. % Purity: 100. Rt: 6.443 min.

### 17-Cyclopropylmethyl-3,14β-dihydroxy-4,5α-epoxy-6α-[(3′-(pyrazolyl-3″-yl)propanamido]morphinan (3)

¹H NMR (400 MHz, DMSO-d₆) δ 9.35 (brs, 2H, exchangeable), 8.87 (brs, 1H, exchangeable), 7.84-7.82 (m, 2H), 6.72 (d, J=8.1 Hz, 1H), 6.55 (d, J=8.1 Hz, 1H), 6.32 (d, J=2.2 Hz, 1H), 4.57 (d, J=3.9 Hz, 1H), 4.43-4.36 (m, 1H), 3.92 (d, J=6.8 Hz, 1H), 3.30-3.21 (m, 2H), 3.07-2.93 (m, 3H), 2.89 (t, J=7.4 Hz, 2H), 2.75-2.65 (m, 1H), 2.56 (t, J=7.3 Hz, 2H), 2.48-2.41 (m, 1H), 1.91-1.83 (m, 1H), 1.62-1.58 (m, 1H), 1.42-1.32 (m, 2H), 1.11-1.04 (m, 1H), 0.98-0.87 (m, 1H), 0.71-0.62 (m, 1H), 0.61-0.57 (m, 1H), 0.51-0.45 (m, 1H), 0.42-0.36 (m, 1H). ¹³C NMR (100 MHz, DMSO-d₆) δ 170.37, 147.14, 146.02, 138.81, 133.66, 128.73, 122.08, 119.04, 118.32, 104.16, 87.47, 69.32, 60.93, 56.97, 45.19, 45.13, 44.90, 33.98, 30.11, 29.15, 23.47, 21.73, 19.71, 5.67, 5.15, 2.54. HRMS calculated for C₂₆H₃₂N₄O₄ m/z: 464.2424. Found [M+H]⁺ (m/z): 465.2477, [M+Na]⁺ (m/z): 487.2296. mp 255.3-256.4° C. dec. % Purity: 100. Rt: 6.708 min.

### 17-Cyclopropylmethyl-3,14/l-dihydroxy-4,5α-epoxy-6α-(4′-pyrazolylcarboxamido)morphinan (4)

¹H NMR (400 MHz, DMSO-d₆) δ 8.85 (brs, 1H, exchangeable), 8.13 (s, 2H), 7.72 (d, J=7.7 Hz, 1H, exchangeable), 6.71 (d, J=8.1 Hz, 1H), 6.57 (d, J=8.1 Hz, 1H), 6.32 (brs, 1H, exchangeable), 4.71 (d, J=3.8 Hz, 1H), 4.60-4.52 (m, 2H), 3.92 (d, J=6.8 Hz, 1H), 3.32-3.24 (m, 2H), 3.10-3.03 (m, 2H), 2.98-2.92 (m, 1H), 2.77-2.67 (m, 1H), 2.47-2.43 (m, 1H), 1.95-1.87 (m, 1H), 1.61 (dd, J=12.8, 1.9 Hz, 1H), 1.49-1.41 (m, 2H), 1.18-1.13 (m, 1H), 0.72-0.66 (m, 1H), 0.64-0.58 (m, 1H), 0.52-0.46 (m, 1H), 0.42-0.37 (m, 1H). ¹³C NMR (100 MHz, DMSO-d₆) δ 159.25, 146.88, 143.43, 136.01, 131.73, 126.20, 119.63, 116.65, 115.64, 114.95, 84.97, 66.76, 62.38, 58.43, 54.43, 42.62, 27.70, 26.60, 20.92, 16.79, 12.58, 3.12, 2.68. HRMS calculated for C₂₄H₂₈N₄O₄ m/z: 436.2111. Found [M+H]⁺ (m/z): 437.2181. mp 259.1-261.2° C. dec. % Purity: 98.45. Rt: 6.261 min.

### 17-Cyclopropylmethyl-3,14β-dihydroxy-4,5α-epoxy-6α-(4′-pyrazolylacetamido) morphinan (5)

¹H NMR (400 MHz, DMSO-d₆) δ 8.86 (brs, 1H, exchangeable), 7.94 (d, J=8.0 Hz, 1H, exchangeable), 7.72 (s, 2H), 6.73 (d, J=8.1 Hz, 1H), 6.56 (d, J=8.1 Hz, 1H), 6.33 (brs, 2H, exchangeable), 4.58 (d, J=3.9 Hz, 1H), 4.42-4.35 (m, 1H), 3.92 (d, J=7.0 Hz, 1H), 3.39 (s, 2H), 3.31-3.22 (m, 2H), 3.08-3.01 (m, 2H), 2.98-2.92 (m, 1H), 2.75-2.65 (m, 1H), 2.47-2.41 (m, 1H), 1.91-1.83 (m, 1H), 1.62-1.58 (m, 1H), 1.42-1.36 (m, 2H), 1.06-1.03 (m, 1H), 0.98-0.89 (m, 1H), 0.71-0.65 (m, 1H), 0.63-0.57 (m, 1H), 0.51-0.45 (m, 1H), 0.41-0.36 (m, 1H). ¹³C NMR (100 MHz, DMSO-d₆) δ 169.61, 146.02, 138.83, 132.82, 128.74, 122.07, 119.05, 118.29, 87.39, 69.31, 60.93, 56.96, 48.55, 45.18, 45.17, 45.12, 45.07, 30.94, 30.12, 29.17, 23.46, 19.65, 5.67, 5.14, 2.53. HRMS calculated for C₂₅H₃₀N₄O₄ m/z: 450.2267. Found [M+H]⁺ (m/z): 451.2351, [M+Na]⁺ (m/z): 473.2165. mp 252.5-253.6° C. dec. % Purity: 100. Rt: 6.337 min.

### 17-Cyclopropylmethyl-3,14β-dihydroxy-4,5α-epoxy-6α-[(3′-(pyrazolyl-4″-yl)propanamido]morphinan (6)

¹H NMR (400 MHz, DMSO-d₆) δ 9.16 (brs, 2H, exchangeable), 8.87 (brs, 1H, exchangeable), 7.75 (d, J=8.0 Hz, 1H, exchangeable), 7.71 (s, 2H), 6.72 (d, J=8.1 Hz, 1H), 6.55 (d, J=8.1 Hz, 1H), 4.56 (d, J=3.8 Hz, 1H), 4.44-4.36 (m, 1H), 3.92 (d, J=6.8 Hz, 1H), 3.35-3.22 (m, 2H), 3.07-2.93 (m, 3H), 2.74-2.68 (m, 3H), 2.46-2.42 (m, 3H), 1.91-1.82 (m, 1H), 1.62-1.58 (m, 1H), 1.41-1.30 (m, 2H), 1.11-1.04 (m, 1H), 0.97-0.86 (m, 1H), 0.71-0.65 (m, 1H), 0.64-0.57 (m, 1H), 0.51-0.45 (m, 1H), 0.42-0.36 (m, 1H). ¹³C NMR (100 MHz, DMSO-d₆) δ 170.88, 146.03, 138.79, 132.06, 128.73, 122.08, 119.83, 119.04, 118.32, 87.53, 69.31, 60.95, 56.97, 45.19, 45.13, 44.80, 36.04, 30.11, 29.17, 23.46, 19.74, 19.65, 5.67, 5.14, 2.54. HRMS calculated for C₂₆H₃₂N₄O₄ m/z: 464.2424. Found [M+H]⁺ (m/z): 465.2511, [M+Na]⁺ (m/z): 487.2326. mp 260.8-261.5° C. dec. % Purity: 95.42. Rt: 6.658 min.

### 17-Cyclopropylmethyl-3,14β-dihydroxy-4,5α-epoxy-6α-(5′-imidazolylcarboxamido) morphinan (7)

¹H NMR (400 MHz, DMSO-d₆) δ 9.24 (brs, 1H, exchangeable), 8.91 (brs, 2H, including 1H exchangeable), 8.49 (brs, 1H, exchangeable), 8.37 (s, 1H), 6.73 (d, J=8.1 Hz, 1H), 6.58 (d, J=8.1 Hz, 1H), 6.43 (brs, 1H, exchangeable), 4.70 (d, J=3.8 Hz, 1H), 4.65-4.58 (m, 1H), 3.97 (d, J=6.8 Hz, 1H), 3.41-3.35 (m, 1H), 3.33-3.23 (m, 2H), 3.10-3.03 (m, 2H), 3.01-2.96 (m, 1H), 2.76-2.67 (m, 1H), 2.55-2.45 (m, 1H), 1.98-1.90 (m, 1H), 1.65-1.62 (m, 1H), 1.55-1.42 (m, 2H), 1.18-1.12 (m, 1H), 0.72-0.66 (m, 1H), 0.64-0.58 (m, 1H), 0.52-0.46 (m, 1H), 0.43-0.37 (m, 1H). ¹³C NMR (100 MHz, DMSO-d₆) δ 169.84, 157.24, 145.86, 138.62, 135.61, 128.60, 122.17, 120.47, 119.31, 118.31, 87.04, 69.22, 60.79, 56.91, 45.50, 45.19, 45.09, 30.10, 28.96, 23.43, 19.31, 5.64, 5.19, 2.51. HRMS calculated for C₂₄H₂₈N₄O₄ m/z: 436.2111. Found [M+H]⁺ (m/z): 437.2167. mp 263.7-264.9° C. dec. % Purity: 99.36. Rt: 6. 970 min.

### 17-Cyclopropylmethyl-3,14β-dihydroxy-4,5α-epoxy-6α-(5′-imidazolylacetamido) morphinan (8)

¹H NMR (400 MHz, DMSO-d₆) δ 14.37 (brs, 2H, exchangeable), 9.28 (s, 1H, exchangeable), 9.01 (d, J=1.2 Hz, 1H), 8.85 (brs, 1H, exchangeable), 8.24 (d, J=8.0 Hz, 1H, exchangeable), 7.48 (s, 1H), 6.74 (d, J=8.1 Hz, 1H), 6.57 (d, J=8.1 Hz, 1H), 6.33 (s, 1H, exchangeable), 4.59 (d, J=3.9 Hz, 1H), 4.45-4.38 (m, 1H), 3.92 (d, J=6.8 Hz, 1H), 3.71 (s, 2H), 3.26-3.22 (m, 2H), 3.08-3.01 (m, 2H), 2.98-2.93 (m, 1H), 2.72-2.66 (m, 1H), 2.45-2.40 (m, 1H), 1.92-1.84 (m, 1H), 1.60 (dd, J=12.8, 2.1 Hz, 1H), 1.46-1.37 (m, 2H), 1.11-1.03 (m, 1H), 1.01-0.92 (m, 1H), 0.71-0.65 (m, 1H), 0.63-0.57 (m, 1H), 0.51-0.45 (m, 1H), 0.42-0.36 (m, 1H). ¹³C NMR (100 MHz, DMSO-d₆) δ 166.82, 145.73, 138.60, 133.49, 128.65, 127.60, 122.09, 119.25, 118.04, 116.83, 87.22, 69.20, 60.82, 56.91, 48.44, 45.27, 45.07, 30.74, 30.07, 28.93, 23.37, 19.40, 5.60, 5.17, 2.45. HRMS calculated for C₂₅H₃₀N₄O₄ m/z: 450.2267. Found [M+H]⁺ (m/z): 451.2360, [M+Na]⁺ (m/z): 473.2178. mp 288.2-290.1° C. dec. % Purity: 96.87. Rt: 6.859 min.

### 17-Cyclopropylmethyl-3,14β-dihydroxy-4,5α-epoxy-6α-[(3′-(imidazol-5″-yl)propanamido]morphinan (9)

¹H NMR (400 MHz, DMSO-d₆) δ 14.33 (brs, 1H, exchangeable), 9.24 (brs, 1H, exchangeable), 8.88 (d, J=1.2 Hz, 1H), 7.91 (d, J=8.1 Hz, 1H, exchangeable), 7.36 (d, J=0.7 Hz, 1H), 6.72 (d, J=8.1 Hz, 1H), 6.55 (d, J=8.2 Hz, 1H), 6.32 (s, 1H, exchangeable), 4.55 (d, J=4.0 Hz, 1H), 4.43-4.35 (m, 1H), 3.91 (d, J=6.8 Hz, 1H), 3.35-3.30 (m, 2H), 3.26-3.21 (m, 1H), 3.06-3.00 (m, 2H), 2.98-2.93 (m, 1H), 2.88 (t, J=7.3 Hz, 2H), 2.72-2.65 (m, 1H), 2.57 (t, J=7.3 Hz, 2H), 2.45-2.40 (m, 1H), 1.91-1.82 (m, 1H), 1.59 (dd, J=12.8, 2.0 Hz, 1H), 1.41-1.32 (m, 2H), 0.98-0.86 (m, 1H), 0.71-0.64 (m, 1H), 0.63-0.57 (m, 1H), 0.50-0.44 (m, 1H), 0.41-0.35 (m, 1H). ¹³C NMR (100 MHz, DMSO-d₆) δ 170.09, 145.91, 138.60, 133.20, 132.65, 128.71, 122.16, 119.13, 118.23, 115.34, 87.41, 69.21, is 60.83, 56.93, 48.43, 45.07, 44.86, 33.37, 30.07, 29.03, 23.43, 20.15, 19.59, 5.64, 5.16, 2.52. HRMS calculated for C₂₆H₃₂N₄O₄ m/z: 464.2424. Found [M+H]⁺ (m/z): 465.2519. mp 286.9-288.4° C. dec. % Purity: 97.03. Rt: 6.692 min.

### 17-Cyclopropylmethyl-3,14β-dihydroxy-4,5α-epoxy-6α-(2′-imidazolylcarboxamido) morphinan (10)

¹H NMR (400 MHz, DMSO-d₆) δ 8.91 (brs, 1H, exchangeable), 8.59-8.46 (m, 1H, exchangeable), 7.62 (s, 1H), 7.56 (s, 1H), 6.73 (d, J=8.1 Hz, 1H), 6.59 (d, J=8.2 Hz, 1H), 6.42 (brs, 1H, exchangeable), 4.75 (d, J=3.9 Hz, 1H), 4.64-4.59 (m, 1H), 3.98-3.97 (m, 1H), 3.38-3.23 (m, 2H), 3.11-3.04 (m, 2H), 3.01-2.97 (m, 1H), 2.76-2.67 (m, 1H), 2.54-2.44 (m, 1H), 1.98-1.89 (m, 1H), 1.66-1.58 (m, 2H), 1.52-1.45 (m, 1H), 1.11-1.07 (m, 2H), 0.72-0.66 (m, 1H), 0.65-0.58 (m, 1H), 0.52-0.46 (m, 1H), 0.43-0.37 (m, 1H). ¹³C NMR (100 MHz, DMSO-d₆) δ 153.76, 145.60, 138.65, 137.81, 128.55, 122.25, 122.17, 119.52, 118.27, 86.84, 69.20, 60.76, 56.92, 48.43, 45.84, 45.23, 45.12, 29.95, 28.83, 23.42, 19.49, 5.63, 5.19, 2.50. HRMS calculated for C₂₄H₂₈N₄O₄ m/z: 436.2111. Found [M+H]⁺ (m/z): 437.2196. mp 268.4-270.2° C. dec. % Purity: 98.60. Rt: 6.675 min.

### 17-Cyclopropylmethyl-3,14β-dihydroxy-4,5α-epoxy-6α-(2′-imidazolylacetamido) morphinan (11)

¹H NMR (400 MHz, DMSO-d₆) δ 14.31 (s, 2H, exchangeable), 9.30 (s, 1H, exchangeable), 8.87 (brs, 1H, exchangeable), 8.50 (d, J=8.0 Hz, 1H, exchangeable), 7.58 (s, 2H), 6.75 (d, J=8.1 Hz, 1H), 6.57 (d, J=8.2 Hz, 1H), 6.38 (brs, 1H, exchangeable), 4.59 (d, J=3.9 Hz, 1H), 4.47-4.39 (m, 1H), 4.08 (s, 2H), 3.93 (d, J=6.8 Hz, 1H), 3.31-3.22 (m, 2H), 3.08-3.01 (m, 2H), 2.98-2.93 (m, 1H), 2.75-2.66 (m, 1H), 2.45-2.40 (m, 1H), 1.93-1.85 (m, 1H), 1.63-1.60 (m, 1H), 1.48-1.37 (m, 2H), 1.10-1.05 (m, 1H), 1.02-0.93 (m, 1H), 0.71-0.65 (m, 1H), 0.63-0.57 (m, 1H), 0.51-0.45 (m, 1H), 0.42-0.36 (m, 1H). ¹³C NMR (100 MHz, DMSO-d₆) δ 162.08, 143.28, 138.88, 136.15, 126.15, 119.64, 116.79, 116.34, 115.63, 84.72, 66.72, 58.27, 54.41, 43.00, 42.62, 29.53, 29.11, 27.59, 26.43, 20.90, 16.90, 3.13, 2.70. HRMS calculated for C₂₅H₃₀N₄O₄ m/z: 450.2267. Found [M+H]⁺ (m/z): 451.2361. mp 264.1-265.8° C. dec. % Purity: 96.56. Rt: 6.849 min.

### 17-Cyclopropylmethyl-3,14β-dihydroxy-4,5α-epoxy-6α-[(3′-(imidazol-2″-yl)propanamido]morphinan (12)

¹H NMR (400 MHz, DMSO-d₆) δ 14.28 (brs, 2H, exchangeable), 9.23 (brs, 1H, exchangeable), 8.86 (brs, 1H, exchangeable), 7.97 (d, J=8.0 Hz, 1H, exchangeable), 7.52 (s, 2H), 6.73 (d, J=8.1 Hz, 1H), 6.55 (d, J=8.1 Hz, 1H), 6.33 (brs, 1H, exchangeable), 4.55 (d, J=3.9 Hz, 1H), 4.41-4.33 (m, 1H), 3.93 (d, J=6.6 Hz, 1H), 3.28-3.21 (m, 1H), 3.12 (t, J=7.1 Hz, 2H), 3.06-2.95 (m, 4H), 2.77 (t, J=7.0 Hz, 2H), 2.72-2.65 (m, 1H), 2.47-2.40 (m, 1H), 1.91-1.83 (m, 1H), 1.60-1.57 (m, 1H), 1.41-1.35 (m, 2H), 1.08-1.04 (m, 1H), 0.96-0.87 (m, 1H), 0.71-0.64 (m, 1H), 0.63-0.57 (m, 1H), 0.51-0.45 (m, 1H), 0.41-0.37 (m, 1H). ¹³C NMR (100 MHz, DMSO-d₆) δ 167.10, 144.34, 143.29, 136.05, 126.22, 119.70, 116.80, 116.02, 115.87, 115.60, 84.87, 66.72, 58.31, 54.45, 45.99, 42.59, 42.52, 29.09, 27.58, 26.50, 20.92, 18.68, 18.16, 16.96, 3.15, 2.75. HRMS calculated for C₂₆H₃₂N₄O₄ m/z: 464.2424. Found [M+H]⁺ (m/z): 465.2510, [M+Na]⁺ (m/z): 487.2321. mp 279.0-281.4° C. dec. % Purity: 98.12. Rt: 7.021 min.

### 17-Cyclopropylmethyl-3,14β-dihydroxy-4,5α-epoxy-6β-(3′-pyrazolylcarboxamido) morphinan (13)

¹H NMR (400 MHz, DMSO-d₆) δ 9.33 (brs, 1H, exchangeable), 8.86 (s, 1H, exchangeable), 8.39 (d, J=8.3 Hz, 1H, exchangeable), 7.76 (d, J=1.8 Hz, 1H), 6.73 (d, J=8.1 Hz, 1H), 6.68 (d, J=1.9 Hz, 1H), 6.65 (d, J=8.2 Hz, 1H), 6.19 (brs, 1H, exchangeable), 4.90 (d, J=7.7 Hz, 1H), 3.87 (d, J=5.1 Hz, 1H), 3.67-3.62 (m, 2H), 3.36-3.31 (m, 2H), 3.11 (d, J=5.9 Hz, 1H), 3.06-3.03 (m, 1H), 2.89-2.85 (m, 1H), 2.47-2.44 (m, 1H), 1.97-1.88 (m, 1H), 1.77-1.74 (m, 1H), 1.57-1.53 (m, 1H), 1.46-1.38 (m, 2H), 1.12-1.04 (m, 1H), 0.70-0.66 (m, 1H), 0.63-0.59 (m, 1H), 0.54-0.49 (m, 1H), 0.45-0.40 (m, 1H). ¹³C NMR (100 MHz, DMSO-d₆) δ 158.66, 139.53, 138.57, 128.71, 127.14, 118.12, 116.86, 115.32, 102.64, 87.24, 67.12, 59.06, 54.11, 47.77, 45.95, 43.84, 43.06, 39.69, 26.93, 24.73, 21.14, 20.36, 3.11, 2.63. HRMS calculated for C₂₄H₂₈N₄O₄ m/z: 436.2111. Found [M+H]⁺ (m/z): 437.2193, [M+Na]⁺ (m/z): 459.2004. mp 268.7-270.2° C. dec. % Purity: 99.99. Rt: 6.197 min.

### 17-Cyclopropylmethyl-3,14β-dihydroxy-4,5α-epoxy-6β-(3′-pyrazolylacetamido) morphinan (14)

¹H NMR (400 MHz, DMSO-d₆) δ 8.87 (brs, 1H, exchangeable), 8.44 (d, J=7.4 Hz, 1H, exchangeable), 7.75 (s, 1H), 6.72 (d, J=8.1 Hz, 1H), 6.63 (d, J=8.1 Hz, 1H), 6.28 (s, 1H), 4.60 (d, J=7.8 Hz, 1H), 3.86 (d, J=5.1 Hz, 1H), 3.59-3.54 (m, 2H), 3.44-3.36 (m, 1H), 3.34-3.27 (m, 2H), 3.08-3.01 (m, 2H), 2.89-2.83 (m, 1H), 2.46-2.37 (m, 2H), 1.81-1.70 (m, 2H), 1.53-1.49 (m, 1H), 1.43-1.41 (m, 1H), 1.36-1.29 (m, 1H), 1.11-1.04 (m, 1H), 0.70-0.66 (m, 1H), 0.62-0.55 (m, 1H), 0.54-0.48 (m, 1H), 0.43-0.37 (m, 1H). ¹³C NMR (100 MHz, DMSO-d₆) δ 168.28, 142.73, 141.95, 141.04, 132.49, 129.53, 120.60, 119.37, 117.82, 104.86, 89.76, 69.56, 61.50, 56.61, 50.78, 46.33, 45.52, 34.02, 29.20, 27.22, 23.44, 22.86, 5.60, 5.10, 2.51. HRMS calculated for C₂₅H₃₀N₄O₄ m/z: 450.2267. Found [M+H]⁺ (m/z): 451.2339, [M+Na]⁺ (m/z): 473.2152. mp 250.2-251.4° C. dec. % Purity: 99.20. Rt: 6.401 min.

### 17-Cyclopropylmethyl-3,14β-dihydroxy-4,5α-epoxy-6β-[(3′-(pyrazolyl-3″-yl)propanamido]morphinan (15)

¹H NMR (400 MHz, DMSO-d₆) δ 8.85 (brs, 1H, exchangeable), 8.22 (brs, 1H, exchangeable), 7.74 (d, J=2.0 Hz, 1H), 6.72 (d, J=8.1 Hz, 1H), 6.63 (d, J=8.1 Hz, 1H), 6.22 (d, J=2.0 Hz, 1H), 4.55 (d, J=7.8 Hz, 1H), 3.85 (brs, 1H), 3.44-3.38 (m, 1H), 3.34-3.27 (m, 2H), 3.08-3.01 (m, 2H), 2.90-2.84 (m, 3H), 2.47-2.32 (m, 4H), 1.72-1.65 (m, 2H), 1.51-1.41 (m, 2H), 1.36-1.28 (m, 1H), 1.10-1.02 (m, 1H), 0.70-0.64 (m, 1H), 0.62-0.55 (m, 1H), 0.53-0.47 (m, 1H), 0.43-0.36 (m, 1H). ¹³C NMR (100 MHz, DMSO-d₆) δ 170.95, 147.52, 142.59, 141.78, 134.15, 130.14, 121.05, 119.68, 118.38, 104.69, 90.37, 70.17, 62.03, 57.13, 51.15, 46.96, 46.03, 34.97, 29.78, 27.78, 24.13, 23.48, 22.13, 6.21, 5.59, 3.10. HRMS calculated for C₂₆H₃₂N₄O₄ m/z: 464.2424. Found [M+H]⁺ (m/z): 465.2503, [M+Na]⁺ (m/z): 487.2322. mp 216.8-217.7° C. dec. % Purity: 100. Rt: 6.812 min.

### 17-Cyclopropylmethyl-3,14β-dihydroxy-4,5α-epoxy-6β-(4′-pyrazolylcarboxamido) morphinan (16)

¹H NMR (400 MHz, DMSO-d₆) δ 8.86 (brs, 1H, exchangeable), 8.26 (d, J=8.1 Hz, 1H, exchangeable), 8.04 (s, 2H), 6.72 (d, J=8.1 Hz, 1H), 6.65 (d, J=8.2 Hz, 1H), 6.18 (brs, 1H, exchangeable), 4.73 (d, J=7.9 Hz, 1H), 3.86 (d, J=5.0 Hz, 1H), 3.66-3.57 (m, 1H), 3.35-3.31 (m, 2H), 3.11-3.03 (m, 2H), 2.87-2.83 (m, 1H), 2.45-2.40 (m, 2H), 1.85-1.73 (m, 2H), 1.59-1.55 (m, 1H), 1.48-1.36 (m, 2H), 1.10-1.04 (m, 1H), 0.69-0.64 (m, 1H), 0.62-0.55 (m, 1H), 0.54-0.48 (m, 1H), 0.44-0.38 (m, 1H). ¹³C NMR (100 MHz, DMSO-d₆) δ 161.78, 142.03, 141.10, 133.90, 129.60, 120.61, 119.32, 117.80, 117.72, 89.87, 69.60, 61.58, 56.61, 50.25, 48.44, 46.38, 45.53, 29.28, 27.25, 23.89, 22.93, 5.63, 5.12, 2.53. HRMS calculated for C₂₄H₂₈N₄O₄ m/z: 436.2111. Found [M+H]⁺ (m/z): 437.2172, [M+Na]⁺ (m/z): 459.1988. mp 275.9-277.1° C. dec. % Purity: 98.78. Rt: 6.332 min.

### 17-Cyclopropylmethyl-3,14β-dihydroxy-4,5α-epoxy-6β-(4′-pyrazolylacetamido) morphinan (17)

¹H NMR (400 MHz, DMSO-d₆) δ 8.84 (brs, 1H, exchangeable), 8.21 (d, J=8.0 Hz, 1H, exchangeable), 7.59 (s, 2H), 6.71 (d, J=8.1 Hz, 1H), 6.63 (d, J=8.2 Hz, 1H), 6.26 (brs, 1H, exchangeable), 4.58 (d, J=7.8 Hz, 1H), 3.84 (d, J=5.3 Hz, 1H), 3.44-3.32 (m, 2H), 3.30-3.25 (m, 3H), 3.08-2.97 (m, 2H), 2.88-2.82 (m, 1H), 2.46-2.36 (m, 2H), 1.78-1.68 (m, 2H), 1.52-1.46 (m, 1H), 1.44-1.41 (m, 1H), 1.36-1.24 (m, 1H), 1.09-1.01 (m, 1H), 0.70-0.64 (m, 1H), 0.61-0.55 (m, 1H), 0.52-0.48 (m, 1H), 0.43-0.37 (m, 1H). ¹³C NMR (100 MHz, DMSO-d₆) δ 169.87, 165.97, 142.00, 141.07, 132.75, 129.56, 120.58, 119.30, 117.81, 114.26, 89.81, 69.57, 61.50, 56.60, 50.65, 46.35, 45.50, 31.23, 29.22, 27.24, 23.51, 22.89, 5.63, 5.10, 2.54. HRMS calculated for C₂₅H₃₀N₄O₄ m/z: 450.2267. Found [M+H]⁺ (m/z): 451.2339, [M+Na]⁺ (m/z): 473.2159. mp 231.8-233.2° C. dec. % Purity: 96.02. Rt: 6.338 min.

### 17-Cyclopropylmethyl-3,14β-dihydroxy-4,5α-epoxy-6β-[(3′-(pyrazolyl-4″-yl)propanamido]morphinan (18)

¹H NMR (400 MHz, DMSO-d₆) δ 8.87 (brs, 1H, exchangeable), 8.18 (d, J=7.9 Hz, 1H, exchangeable), 7.70 (s, 2H), 6.73 (d, J=8.1 Hz, 1H), 6.63 (d, J=8.2 Hz, 1H), 6.26 (brs, 2H, exchangeable), 4.54 (d, J=7.9 Hz, 1H), 3.87 (d, J=4.9 Hz, 1H), 3.45-3.37 (m, 1H), 3.33-3.26 (m, 2H), 3.08-3.01 (m, 2H), 2.90-2.84 (m, 1H), 2.71-2.67 (m, 2H), 2.46-2.40 (m, 2H), 2.34 (t, J=7.5 Hz, 2H), 1.74-1.64 (m, 2H), 1.48-1.40 (m, 2H), 1.35-1.28 (m, 1H), 1.11-1.04 (m, 1H), 0.70-0.64 (m, 1H), 0.62-0.54 (m, 1H), 0.53-0.48 (m, 1H), 0.43-0.37 (m, 1H). ¹³C NMR (100 MHz, DMSO-d₆) δ 171.36, 141.99, 141.02, 132.13, 129.57, 120.63, 119.58, 119.34, 117.84, 89.89, 69.57, 61.48, 56.60, 50.38, 46.31, 45.48, 36.55, 29.24, 27.20, 23.52, 22.86, 19.51, 5.60, 5.10, 2.51. HRMS calculated for C₂₆H₃₂N₄O₄ m/z: 464.2424. Found [M+H]⁺ (m/z): 465.2490. mp 229.8-231.0° C. dec. % Purity: 99.85. Rt: 6.526 min.

### 17-Cyclopropylmethyl-3,14β-dihydroxy-4,5α-epoxy-6β-(5′-imidazolylcarboxamido) morphinan (19)

¹H NMR (400 MHz, DMSO-d₆) δ 9.36 (brs, 1H, exchangeable), 9.14 (brs, 1H, exchangeable), 8.88 (brs, 1H, exchangeable), 8.80 (brs, 1H), 8.20 (s, 1H), 6.73 (d, J=8.1 Hz, 1H), 6.65 (d, J=8.2 Hz, 1H), 6.26 (s, 1H, exchangeable), 4.84 (d, J=7.8 Hz, 1H), 3.89 (d, J=5.4 Hz, 1H), 3.70-3.61 (m, 2H), 3.36-3.33 (m, 1H), 3.10 (d, J=5.9 Hz, 1H), 3.06-3.03 (m, 1H), 2.89-2.85 (m, 1H), 2.46-2.40 (m, 2H), 1.95-1.85 (m, 1H), 1.80-1.77 (m, 1H), 1.60-1.56 (m, 1H), 1.46-1.36 (m, 2H), 1.11-1.05 (m, 1H), 0.71-0.65 (m, 1H), 0.62-0.56 (m, 1H), 0.54-0.48 (m, 1H), 0.44-0.38 (m, 1H). ¹³C NMR (100 MHz, DMSO-d₆) δ 157.07, 141.89, 141.22, 135.61, 129.52, 128.36, 120.65, 120.19, 119.45, 117.86, 89.50, 69.56, 61.45, 56.61, 50.79, 46.36, 45.60, 29.33, 27.19, 23.58, 22.93, 5.66, 5.12, 2.57. HRMS calculated for C₂₄H₂₈N₄O₄ m/z: 436.2111. Found [M+H]⁺ (m/z): 437.2175, [M+Na]⁺ (m/z): 459.1992. mp 270.8-272.5° C. dec. % Purity: 99.62. Rt: 6.885 min.

### 17-Cyclopropylmethyl-3,14β-dihydroxy-4,5α-epoxy-6β-(5′-imidazolylacetamido) morphinan (20)

¹H NMR (400 MHz, DMSO-d₆) δ 14.37 (brs, 2H, exchangeable), 9.36 (s, 1H, exchangeable), 9.00 (d, J=1.2 Hz, 1H), 8.84 (brs, 1H, exchangeable), 8.59 (d, J=7.9 Hz, 1H, exchangeable), 7.47 (s, 1H), 6.73 (d, J=8.1 Hz, 1H), 6.63 (d, J=8.2 Hz, 1H), 6.26 (s, 1H, exchangeable), 4.60 (d, J=7.8 Hz, 1H), 3.86 (d, J=5.3 Hz, 1H), 3.66 (s, 2H), 3.46-3.37 (m, 2H), 3.27-3.22 (m, 1H), 3.08-3.02 (m, 2H), 2.89-2.84 (m, 1H), 2.44-2.38 (m, 2H), 1.83-1.71 (m, 2H), 1.54-1.51 (m, 1H), 1.44-1.42 (m, 1H), 1.36-1.24 (m, 1H), 1.09-1.05 (m, 1H), 0.69-0.64 (m, 1H), 0.62-0.57 (m, 1H), 0.53-0.48 (m, 1H), 0.43-0.38 (m, 1H). ¹³C NMR (100 MHz, DMSO-d₆) δ 166.88, 142.03, 141.30, 133.64, 129.59, 127.51, 120.58, 119.26, 117.88, 117.04, 89.75, 69.64, 61.55, 56.66, 51.09, 46.46, 45.58, 31.10, 29.26, 27.30, 23.57, 22.98, 5.71, 5.09, 2.61. HRMS calculated for C₂₅H₃₀N₄O₄ m/z: 450.2267. Found [M+H]⁺ (m/z): 451.2345, [M+Na]⁺ (m/z): 473.2158. mp 292.4-294.3° C. dec. % Purity: 98.23. Rt: 6.525 min.

### 17-Cyclopropylmethyl-3,14l-dihydroxy-4,5α-epoxy-6β-[(3′-(imidazol-5″-yl) propanamido]morphinan (21)

¹H NMR (400 MHz, DMSO-d₆) δ 14.50 (brs, 1H, exchangeable), 14.32 (brs, 1H, exchangeable), 9.35 (brs, 1H, exchangeable), 8.98 (d, J=1.0 Hz, 1H), 8.85 (brs, 1H, exchangeable), 8.32 (d, J=7.9 Hz, 1H, exchangeable), 7.38 (s, 1H), 6.73 (d, J=8.1 Hz, 1H), 6.63 (d, J=8.2 Hz, 1H), 6.26 (brs, 1H, exchangeable), 4.55 (d, J=7.8 Hz, 1H), 3.87 (d, J=5.0 Hz, 1H), 3.33-3.26 (m, 4H), 3.07-3.02 (m, 2H), 2.87 (t, J=7.2 Hz, 3H), 2.46-2.38 (m, 3H), 1.76-1.67 (m, 2H), 1.48-1.40 (m, 2H), 1.34-1.27 (m, 1H), 1.11-1.04 (m, 1H), 0.68-0.64 (m, 1H), 0.62-0.57 (m, 1H), 0.54-0.49 (m, 1H), 0.43-0.38 (m, 1H). ¹³C NMR (100 MHz, DMSO-d₆) δ 170.11, 142.09, 141.28, 133.27, 132.54, 129.64, 120.59, 119.20, 117.89, 115.43, 89.87, 69.66, 61.48, 56.62, 50.64, 46.45, 45.53, 33.90, 29.30, 27.26, 23.64, 22.98, 20.04, 5.72, 5.10, 2.61. HRMS calculated for C₂₆H₃₂N₄O₄ m/z: 464.2424. Found [M+H]⁺ (m/z): 465.2516, [M+Na]⁺ (m/z): 487.2334. mp 278.7-280.9° C. dec. % Purity: 99.23. 1o Rt: 6.977 min.

### 17-Cyclopropylmethyl-3,14β-dihydroxy-4,5α-epoxy-6β-(2′-imidazolylcarboxamido) morphinan (22)

¹H NMR (400 MHz, DMSO-d₆) δ 9.53 (brs, 1H, exchangeable), 9.33 (brs, 1H, exchangeable), 8.85 (brs, 1H, exchangeable), 7.56 (s, 2H), 6.73 (d, J=8.1 Hz, 1H), 6.66 (d, J=8.2 Hz, 1H), 6.22 (brs, 1H, exchangeable), 4.87 (d, J=7.8 Hz, 1H), 3.86 (d, J=5.3 Hz, 1H), 3.72-3.63 (m, 1H), 3.36-3.29 (m, 2H), 3.13-3.03 (m, 2H), 2.88-2.84 (m, 1H), 2.46-2.40 (m, 1H), 1.98-1.87 (m, 1H), 1.79-1.76 (m, 1H), 1.63-1.59 (m, 1H), 1.50-1.39 (m, 2H), 1.06-1.02 (m, 1H), 0.71-0.65 (m, 1H), 0.63-0.56 (m, 1H), 0.54-0.48 (m, 1H), 0.44-0.38 (m, 1H). ¹³C NMR (100 MHz, DMSO-d₆) δ 156.82, 141.95, 141.14, 139.86, 129.58, 123.69, 120.63, 119.41, 117.85, 89.61, 69.59, 61.50, 56.61, 50.58, 46.35, 45.60, 29.45, 27.22, 23.52, 22.89, 5.63, 5.11, 2.54. HRMS calculated for C₂₄H₂₈N₄O₄ m/z: 436.2111. Found [M+H]⁺ (m/z): 437.2177. mp 270.7-272.4° C. dec. % Purity: 96.44. Rt: 6.439 min.

### 17-Cyclopropylmethyl-3,14β-dihydroxy-4,5α-epoxy-6β-(2′-imidazolylacetamido) morphinan (23)

¹H NMR (400 MHz, DMSO-d₆) δ 14.17 (brs, 2H, exchangeable), 9.35 (s, 1H, exchangeable), 8.81 (d, J=7.7 Hz, 2H, exchangeable), 7.57 (s, 2H), 6.72 (d, J=8.1 Hz, 1H), 6.64 (d, J=8.1 Hz, 1H), 6.20 (s, 1H, exchangeable), 4.59 (d, J=7.8 Hz, 1H), 4.00 (s, 2H), 3.84 (d, J=5.4 Hz, 1H), 3.45-3.37 (m, 3H), 3.08-3.02 (m, 2H), 2.88-2.82 (m, 1H), 2.46-2.42 (m, 2H), 1.82-1.70 (m, 2H), 1.58-1.54 (m, 1H), 1.45-1.43 (m, 1H), 1.38-1.30 (m, 1H), 1.11-1.04 (m, 1H), 0.71-0.65 (m, 1H), 0.62-0.55 (m, 1H), 0.53-0.47 (m, 1H), 0.43-0.37 (m, 1H). ¹³C NMR (100 MHz, DMSO-d₆) δ 164.61, 141.89, 141.17, 129.50, 120.95, 120.62, 119.41, 118.88, 117.85, 89.60, 69.52, 61.44, 56.62, 51.23, 46.35, 45.57, 32.15, 29.22, 27.25, 23.46, 22.92, 5.66, 5.12, 2.58. HRMS calculated for C₂₅H₃₀N₄O₄ m/z: 450.2267. Found [M+H]⁺ (m/z): 451.2320, [M+Na]⁺ (m/z): 473.2133. mp 275.6-277.0° C. dec. % Purity: 97.80. Rt: 6.210 min.

### 17-Cyclopropylmethyl-3,14β-dihydroxy-4,5α-epoxy-6β-[(3′-(imidazol-2″-yl)propanamido]morphinan (24)

¹H NMR (400 MHz, DMSO-d₆) δ 14.28 (s, 2H, exchangeable), 9.38 (brs, 1H, exchangeable), 8.86 (brs, 1H, exchangeable), 8.41 (d, J=7.9 Hz, 1H, exchangeable), 7.51 (s, 2H), 6.72 (d, J=8.1 Hz, 1H), 6.62 (d, J=8.2 Hz, 1H), 6.27 (s, 1H, exchangeable), 4.56 (d, J=7.8 Hz, 1H), 3.87 (d, J=5.2 Hz, 1H), 3.37-3.26 (m, 3H), 3.12-3.01 (m, 4H), 2.89-2.84 (m, 1H), 2.71 (t, J=7.2 Hz, 2H), 2.45-2.37 (m, 2H), 1.77-1.67 (m, 2H), 1.48-1.40 (m, 2H), 1.32-1.26 (m, 1H), 1.10-1.04 (m, 1H), 0.70-0.63 (m, 1H), 0.61-0.56 (m, 1H), 0.54-0.48 (m, 1H), 0.42-0.37 (m, 1H). ¹³C NMR (100 MHz, DMSO-d₆) δ 169.54, 146.90, 142.03, 141.26, 129.58, 120.55, 119.23, 118.41, 117.89, 89.79, 69.64, 61.53, 56.63, 50.78, 46.44, 45.54, 31.99, 29.26, 27.27, 23.61, 22.96, 21.13, 5.71, 5.09, 2.60. HRMS calculated for C₂₆H₃₂N₄O₄ m/z: 464.2424. Found [M+H]⁺ (m/z): 465.2502, [M+Na]⁺ (m/z): 487.2324. mp 285.1-287.2° C. dec. % Purity: 97.83. Rt: 6.984 min.

### Biological Evaluation.

Drugs. The free base of naltrexone was provided through NIDA Drug Supply Program. All drugs and test compounds were dissolved in sterile-filtered distilled/deionized water. All other reagents and radioligands were purchased from either Sigma-Aldrich or Perkin-Elmer.

### In Vitro Competitive Radioligand Binding Assay.

The competition binding assay was conducted using the monoclonal mouse opioid mu or kappa receptor expressed in CHO cell lines (monoclonal human δ opioid receptor was used in the DOR assay). In this assay, 30 μg of membrane protein was incubated with the corresponding radioligand in the presence of different concentrations of test compounds in TME buffer (50 mM Tris, 3 mM MgCl₂, and 0.2 mM EGTA, pH 7.4) for 1.5 h at 30° C. The bound radioligand was separated by filtration using the Brandel harvester. Specific (i.e., opioid receptor-related) binding to the MOR, KOR, and DOR was determined as the difference in binding obtained in the absence and presence of 5 μM of naltrexone, U50,488, and SNC80, respectively. Relative affinity values (IC₅₀) were determined by fitting displacement binding inhibition values by non-linear regression using GraphPad Prism 8.0 (GraphPad Software, San Diego, CA), where % inhibition value was calculated as follows: % inhibition=100%−(binding in the presence of tested compound−nonspecific binding)/specific binding×100%. The IC₅₀ values were converted to Ki values using the Cheng-Prusoff equation: Kᵢ=IC₅₀/[1+([L*]/K_{D})], where [L*] is the concentration of the radioligand and K_{D} is the K_{D} of the radioligand.

### In Vitro [³⁵S]GTPγS Functional Assay.

The [³⁵S]GTPγS functional assay was conducted to determine the efficacy of the compounds at the MOR, KOR and DOR. In this assay, 10 g of MOR-CHO/KOR-CHO/DOR-CHO membrane protein was incubated in a final volume of 500 μL containing TME with 100 mM NaCl, 20 μM GDP, 0.1 nM [³⁵S]GTPγS, and varying concentrations of the compound under investigation for 1.5 h in a 30° C. water bath. The Bradford protein assay was utilized to determine and adjust the concentration of protein required for the assay. Nonspecific binding was determined with 20 μM unlabeled GTPγS. Furthermore, 3 μM DAMGO/U50488H/SNC80 was included in the assay as the maximally effective concentration of a full agonist for the MOR/KOR/DOR. After incubation, the bound radioactive ligand was separated from the free radioligand by filtration through a GF/B glass fiber filter paper using a Brandel harvester. Bound radioactivity was determined by liquid scintillation counting. All assays were determined in duplicate and repeated at least three times. Net stimulated [³⁵S]GTPγS binding was defined as agonist-stimulated minus basal binding in the absence of agonist. Percent of DAMGO/U50488H/SNC80 stimulated [³⁵S]GTPγS binding was defined as (net-stimulated binding by ligand/net-stimulated binding by 3 μM DAMGO U50488H/SNC80)×100%.

### Animals.

5-8 week 25-35 g male Swiss Webster mice were housed in cages (5 maximal per cage) in animal care quarters and maintained at 22±2° C. on a 12 h light-dark cycle. Food (standard chow) and water were available ad libitum. The mice were brought to the lab (22±2° C., 12 h light-dark cycle) and allowed at least 18 h to recover from transport. Protocols and procedures were approved by the Institutional Animal Care and Use Committee at Virginia Commonwealth University Medical Center and comply with the recommendations of the International Association for the Study of Pain. All mice were used only once.

### Tail-Withdrawal Study.

The tail-withdrawal test was performed using a water bath with the temperature maintained at 56±0.1° C. Baseline latency was measured before any injections. Each mouse was gently wrapped in a cloth with only the tail exposed. The distal one-third of the tail was immersed perpendicularly in water, and the mouse rapidly flicked the tail from the bath was seen as the first sign of discomfort. The duration of time the tail remained in the water bath was counted as the baseline latency. Untreated mice with baseline latency reaction times ranging from 2 to 4 seconds were used. Test latency was obtained 20 min later after each injection. A 10-second maximum cutoff latency was used to prevent any tissue damage. Antinociception was quantified as the percentage of maximal possible effect (% MPE), which was calculated as % MPE=[(test latency−control latency)/(10−control latency)]×100. The % MPE value was calculated for each mouse using 6 mice per group. Testing compounds were given 10 mg/kg (s.c.) to each mouse. In the morphine challenge study, testing compounds or controls were administered 5 min prior to the subcutaneous injection of 10 mg/kg morphine.

### Carmine Red Dye Study.

Each mouse was placed in an individual cage. Five minutes prior to morphine (or vehicle) injection, the testing compound or vehicle was given (s.c. or p.o.) to a group of 5-6 mice. After 20 min of the morphine administration, 0.2 mL red dye solution containing 0.5% carboxymethyl cellulose (CMC) and 6% carmine red dye in ddH₂O was given to each mouse (p.o.) and the time when mouse was fed was recorded as time 0. Then the time which costed each mouse to defecate the first red pellet was measured and recorded. Cut-off time was 6 hours.

### Statistical Analysis.

One-way ANOVA followed by the corrected Dunnett test were performed to assess significance using Prism 8.0 software (GraphPad Software, San Diego, CA).

### Abbreviations

BBB, blood-brain barrier; CHO, Chinese hamster ovary; CMC, carboxymethyl cellulose; CNS, central nervous system; DAMGO, [D-Ala2-MePhe4-Gly(ol)5]enkephalin; DOR, 6 opioid receptor; EDCI, 1-ethyl-3-(3-dimethylaminopropyl)carbodiimide; FCC, flash column chromatography; GI, gastrointestinal; HBD, hydrogen bond donor; HOBt, hydroxybenzotriazole; HPLC, high performance liquid chromatography; KOR, κ opioid receptor; MNTX, methylnaltrexone; MOR, μ opioid receptor; mp, melting points; MPE, maximal possible effect; MPO, multiparameter optimization; MW, molecular weight; NIDA, National Institute of Drug Abuse; NLX, naloxone; NMR, nuclear magnetic resonance; OIC, opioid-induced constipation; PAMORAs, peripherally acting-opioid receptor antagonists; PNS, peripheral nervous system; SARs, structure-activity relationships; s.c., subcutaneously; SEM, standard error of mean; TLC, thin-layer chromatography; TPSA, topological polar surface area.

### Example 3. Synthesis of compound 17-Cyclopropylmethyl-3,14β-dihydroxy-4,5α-epoxy-6α-(2′-thiazolylcarboxamido)morphinan hydrochloride

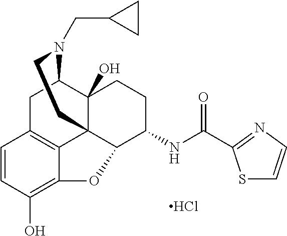

The compound was synthesized according to previously reported procedures (Li, et al. J. Med. Chem. 2009, 52, 1416-1427; Ma, et al. J. Med. Chem. 2019, 62, 11399-11415; Obeng, et al. ACS Chem. Neurosci. 2019, 10, 1075-1090; Yuan, et al. Bioorg. Med. Chem. 2015, 23, 1701-1715). Briefly, 6α-naltrexamine (NTA) was synthesized by stereoselective reduction amination of naltrexone with benzylamine, followed by catalytic hydrogenation under acidic conditions. Various commercially available 5-membered heterocyclic carboxylic acids were coupled with 6α-naltrexamine utilizing the EDCI/HOBt coupling reaction under mild basic conditions. 6-Position monosubstituted free bases were then obtained in reasonable yields by treating with K₂CO₃ in methanol. The final compound, obtained in a yield of 65%, was converted to its hydrochloric acid salt form, fully characterized, and applied for in vitro and in vivo pharmacological characterization.

### 17-Cyclopropylmethyl-3,14β-dihydroxy-4,5α-epoxy-6α-(2′-thiazolylcarboxamido)morphinan hydrochloride

¹H NMR (400 MHz, DMSO-d₆) δ 9.34 (s, 1H, exchangeable), 8.83 (brs, 1H, exchangeable), 8.12-8.09 (m, 2H, including an exchangeable proton), 8.05 (d, J=3.1 Hz, 1H), 6.73 (d, J=8.1 Hz, 1H), 6.60 (d, J=8.1 Hz, 1H), 6.27 (s, 1H, exchangeable), 4.77 (d, J=3.8 Hz, 1H), 4.64-4.57 (m, 1H), 3.89 (dd, J=6.8 Hz, 0.44 Hz, 1H), 3.41-3.37 (m, 1H), 3.30-3.26 (m, 1H), 3.11-3.03 (m, 2H), 2.97-2.90 (m, 1H), 2.78-2.66 (m, 1H), 2.46-2.43 (m, 1H), 1.94-1.85 (m, 1H), 1.69-1.64 (m, 1H), 1.61-1.54 (m, 1H), 1.49-1.43 (m, 1H), 1.09 (t, J=7.0 Hz, 1H), 1.07-1.01 (m, 1H), 0.73-0.67 (m, 1H), 0.65-0.58 (m, 1H), 0.51-0.45 (m, 1H), 0.43-0.37 (m, 1H). ¹³C NMR (100 MHz, DMSO-d₆) δ 163.12, 158.37, 145.71, 143.91, 138.97, 128.63, 126.19, 122.04, 119.44, 118.33, 87.19, 69.27, 60.94, 57.02, 45.61, 45.31, 45.16, 30.09, 29.14, 23.46, 19.73, 5.66, 5.14, 2.55. ESI-HRMS calcd for C₂₄H₂₈N₃O₄S m/z [M+H]⁺454.1795, found 454.1796; calcd for C₂₄H₂₇N₃NaO₄S m/z [M+Na]⁺476.1614, found 476.1603. % Purity: 96.02. Rt: 7.142 min.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

1. A compound having the general formula: 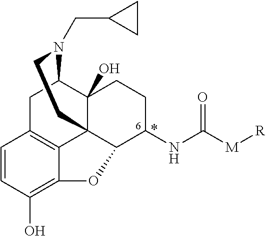
where
R= 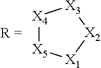
and is saturated, unsaturated, aromatic or heteroaromatic, where X1, X2, X3, X4 and X5 are independently C, N, O or S;
M=a saturated or unsaturated, branched or unbranched, substituted or unsubstituted alkyl chain comprising from 0-10 carbon atoms;
and
* indicates a chiral C with an α or β configuration,
and salts and stereoisomers thereof,
wherein -M-R is not

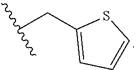 [SMILES: *Cc1cccs1]

2. The compound of claim 1, where M is 0, methyl or ethyl.

3. The compound of claim 1, where R is

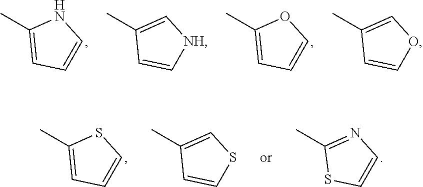

4. The compound of claim 1, where M=0 and R is a 5-membered heterocyclic ring comprising one or more of S, N and NH.

5. The compound of claim 4, wherein the chiral C has an a configuration and the compound is

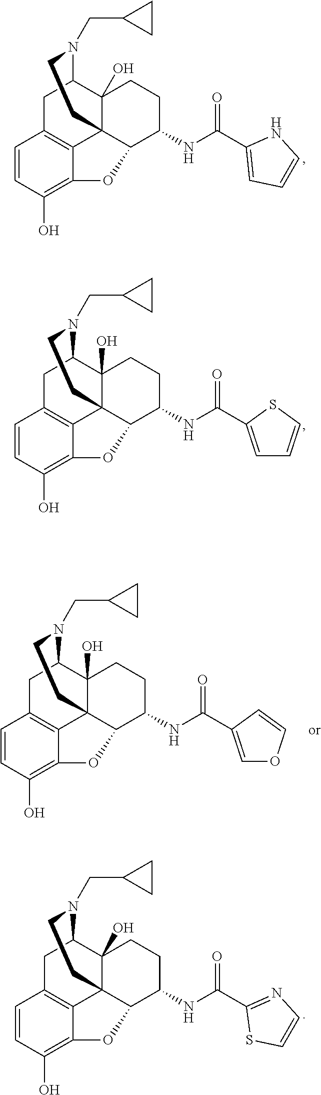

6. The compound of claim 5, wherein the compound is a hydrochloride salt.

7. The compound of claim 1, where M=0 or 1 and R is a 5-membered heterocyclic ring comprising N and NH.

8. The compound of claim 7, where R is

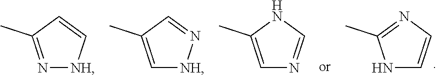

9. The compound of claim 8, wherein
the chiral C has an α configuration and the compound is 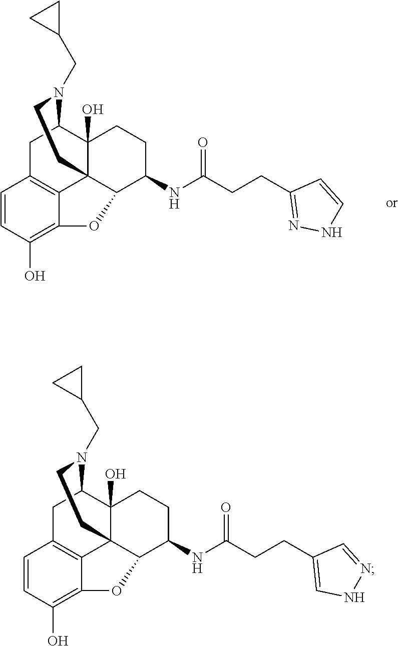
or
the chiral C has a β configuration the compound is

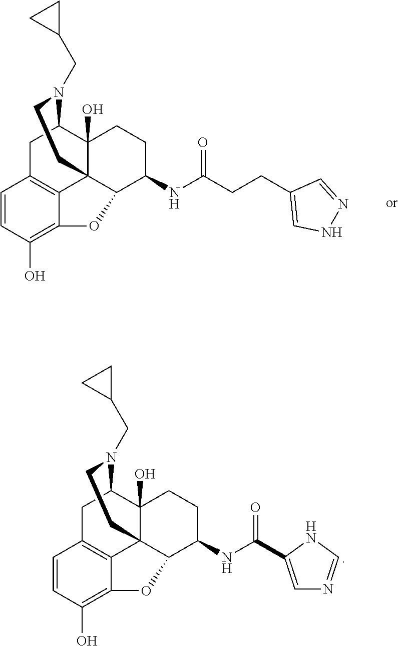

10. A composition comprising
at least one compound of claim 1 and
a physiologically acceptable carrier.

11. A method of preventing or treating opioid addiction in a subject in need thereof, comprising
administering to the subject a therapeutically effective amount of at least one compound of claim 1, wherein the at least one compound is administered to the subject instead of the opioid.

12. The method of claim 11, wherein the at least one compound is

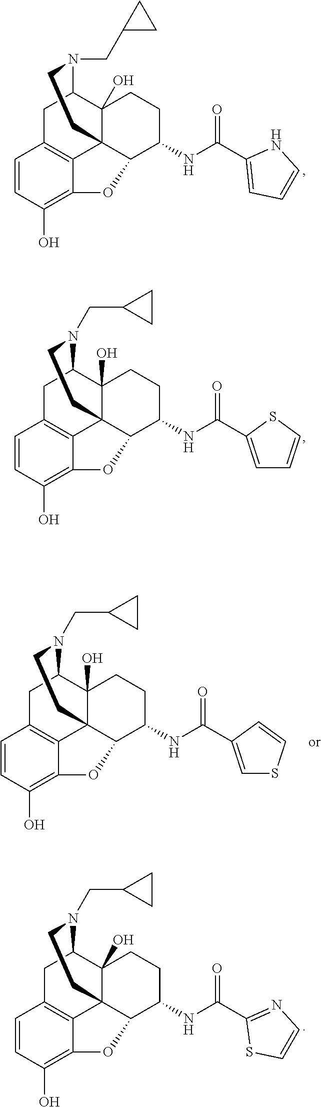

13. The compound of claim 12, wherein the at least one compound is a hydrochloride salt.

14. A method of treating opioid-induced constipation in a subject in need thereof, comprising
administering to the subject a therapeutically efficient among of at least one compound of claim 7.

15. The method of claim 14, wherein the at least one compound is administered in combination with an opioid.

16. The method of claim 14, wherein the at least one compound is administered orally.

17. A method of treating an opioid overdose in a subject in need thereof, comprising
administering to the subject a therapeutically effective amount of at least one compound of claim 1.

18. A composition comprising
an opioid and
a compound of claim 7.
